SEQUENCE LISTING (268 sequences)

SEQ ID NO: 1 - DNA, 253 bp, Artificial Sequence
  Synthetic Polypeptide
tacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacagg

SEQ ID NO: 2 - DNA, 253 bp, Artificial Sequence
  Synthetic Polypeptide
tacgtatggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacagg

SEQ ID NO: 3 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgcagcggtgaaatgcttagatatcacgaagaactccgattgcgaaggcagcctgctaagctgcaactgacattgaggctcgaaagtgtgggtatcaaacagg

SEQ ID NO: 4 - DNA, 253 bp, Artificial Sequence
  Synthetic Polypeptide
tacggaggatccgagcgttatccggatttattgggtttaaagggagcgtagatggatgtttaagtcagttgtgaaagtttgcggcccaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 5 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatattacgaagaactccgattgcgaaggcagcctgctaagctgcaactgacattgaggctcgaaagtgtgggtatcaaacagg

SEQ ID NO: 6 - DNA, 253 bp, Artificial Sequence
  Synthetic Polypeptide
tacggaggatccgagcgttatccggatttattgggtttaaagggagcgtagatggatgtctaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 7 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccggttgcgaaggcagcctgctaagctgcaactgacattgaggctcgaaagtgtgggtatcaaacagg

SEQ ID NO: 8 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgcgggtatcaaacagg

SEQ ID NO: 9 - DNA, 253 bp, Artificial Sequence
  Synthetic Polypeptide
tacggaggatccgagcgttatccggatttattgggtttaaagggagcgtggatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 10 - DNA, 253 bp, Artificial Sequence
  Synthetic Polypeptide
tacgtatggagcaagcgttatccggatttactgggtgtaaagggagtgtaggtggccaggcaagtcagaagtgaaagcccggggctcaaccccgggactgcttttgaaactgcagggctagagtgcaggaggggcaagtggaattcctagtgtagcggtgaaatgcgtagatattaggaggaacaccagtggcgaaggcggcttgctggactgtaactgacactgaggctcgaaagcgtggggagcaaacagg

SEQ ID NO: 11 - DNA, 253 bp, Artificial Sequence
  Synthetic Polypeptide
tacgtatggtgcaagcgttatccggatttactgggtgtaaagggagtgtaggtggccaggcaagtcagaagtgaaagcccggggctcaaccccgggactgcttttgaaactgcagggctagagtgcaggaggggcaagtggaattcctagtgtagcggtgaaatgcgtagatattaggaggaacaccagtggcgaaggcggcttgctggactgtaactgacactgaggctcgaaagcgtggggagcaaacagg

SEQ ID NO: 12 - DNA, 253 bp, Artificial Sequence
  Synthetic Polypeptide
tacgtatggagcaagcgttatccggatttactgggtgtaaagggagtgtaggtggccaggcaagtcagaagtgaaagcccggggctcaaccccgggactgcttttgaaactgcagggctagagtgcaggaggggcaagtggaattcctagtgtagcggtgaaatgcgtagatattaggaggaacaccagtggcgaaggcggcttactggacgataactgacgctgaggctcgaaagcgtggggagcaaacagg

SEQ ID NO: 13 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 14 - DNA, 253 bp, Artificial Sequence
  Synthetic Polypeptide
tacgtatggagcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 15 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacatcagtggcgaaggcggcttactggactgaaactgacactgaggcacgaaagcgtggggagcaaacagg

SEQ ID NO: 16 - DNA, 253 bp, Artificial Sequence
  Synthetic Polypeptide
tacgtatggtgcaagcgttatccggatttactgggcgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 17 - DNA, 253 bp, Artificial Sequence
  Synthetic Polypeptide
tacgtatggtgcaagcgttatccggatttactgggtgtaaagggagcgcaggcggcgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 18 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctggggctcgaaagcgtggggagcaaacagg

SEQ ID NO: 19 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgccggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 20 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaggcgtggggagcaaacagg

SEQ ID NO: 21 - DNA, 253 bp, Artificial Sequence
  Synthetic Polypeptide
aacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 22 - DNA, 253 bp, Artificial Sequence
  Synthetic Polypeptide
aacgtaggtcacaagcgttgtccggaattactgggtgtaaagggagcgcaggcgggaagacaagttggaagtgaaatccatgggctcaacccatgaactgctttcaaaactgtttttcttgagtagtgcggaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 23 - DNA, 253 bp, Artificial Sequence
  Synthetic Polypeptide
aacgtaggtcacaagcgttgtccggaattactgggtgtaaagggagcgcaggcgggaagacaagttggaagtgaaatccatgggctcaacccatgaactgctttcaaaactgcttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 24 - DNA, 253 bp, Artificial Sequence
  Synthetic Polypeptide
aacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcgacctactgggcaccaactgacgctgaggctcgaaagcatgggtagcaaacagg

SEQ ID NO: 25 - DNA, 253 bp, Artificial Sequence
  Synthetic Polypeptide
tacgtaggtggcaagcgttgtccggatttactgggtgtaaagggcgtgtaggcggagatgcaagtcagatgtgaaatcctcgggcttaacccgggaactgcatttgaaactgtatcccttgagtatcggagaggcaggcggaattcctagtgtagcggtgaaatgcgtagatattaggaggaacaccagtggcgaaggcggcctgctggacgacaactgacgctgaggcgcgaaagcgtggggagcaaacagg

SEQ ID NO: 26 - DNA, 253 bp, Artificial Sequence
  Synthetic Polypeptide
tacgtaggtggcaagcgttgtccggatttactgggtgtaaagggcgtgtaggcgggtagacaagtcagatgtgaaataccggggctcaactccggggctgcatttgaaactgtatatcttgagtgtcggagaggaaagcggaattcctagtgtagcggtgaaatgcgtagatattaggaggaacaccagtggcgaaggcggctttctggacgataactgacgctgaggcgcgaaagcgtggggagcaaacagg

SEQ ID NO: 27 - DNA, 253 bp, Artificial Sequence
  Synthetic Polypeptide
tacgtagggggcaagcgttatccggatttactgggtgtaaagggagcgtagacggcacagcaagtctgaagtgaaatccccgggctcaacccgggaactgctttggaaactgttgggctggagtgctggagaggcaagcggaattcctagtgtagcggtgaaatgcgtagatattaggaggaacaccagtggcgaaggcggcttgctggacagtaactgacgttgaggctcgaaagcgtggggagcaaacagg

SEQ ID NO: 28 - DNA, 253 bp, Artificial Sequence
  Synthetic Polypeptide
tacgtaggtggcgagcgttatccggaatgattgggcgtaaagggtgcgtaggtggcagatcaagtctggagtaaaaggtatgggctcaacccgtacttgctctggaaactgatcagctagagaacagaagaggacggcggaactccatgtgtagcggtaaaatgcgtagatatatggaagaacaccggcggcgaaggcggccgtctggtctggattctgacactgaagcacgaaagcgtggggagcaaatagg

SEQ ID NO: 29 - DNA, 252 bp, Artificial Sequence
  Synthetic Polypeptide
tacgtaggtggcgagcgttatccggaatcattgggcgtaaagagggagcaggcggccgcaagggtctgtggtgaaagaccgaagctaaacttcggtaagccatggaaaccgggcggctagagtgcggaagaggatcgtggaattccatgtgtagcggtgaaatgcgtagatatatggaggaacaccagtggcgaaggcgacggtctgggccgcaactgacgctcattcccgaaagcgtggggagcaaacagg

SEQ ID NO: 30 - DNA, 252 bp, Artificial Sequence
  Synthetic Polypeptide
tacgtaggtggcgagcgttatccggagtcattgggcgtaaagagggagcaggcggccgcaagggtctgtggtgaaagaccgaagctaaacttcggtaagccatggaaaccgggcggctagagtgcggaagaggatcgtggaattccatgtgtagcggtgaaatgcgtagatatatggaggaacaccagtggcgaaggcgacggtctgggccgcaactgacgctcattcccgaaagcgtggggagcaaatagg

SEQ ID NO: 31 - DNA, 253 bp, Artificial Sequence
  Synthetic Polypeptide
tacggagggtgcaagcgttaatcggaatcactgggcgtaaagcgcacgtaggcggcccggtaagtcaggggtgaaatcccacagcccaactgtggaactgcccttgatactgccgggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacagg

SEQ ID NO: 32 - DNA, 253 bp, Artificial Sequence
  Synthetic Polypeptide
tacgaagggggcaagcgttgttcggaattactgggcgtaaagggtgtgtaggcggatttgtaagatagtggtgaaatacctgagctcaacttaggaattgccattataactatagatctggagtgacagagaggatattggaatacccagtgtagaggtgaaattcgtagatattgggtagaacaccagtggcgaaggcgagtatctggctgtcaactgacgctgaggcacgaaagcatggggatcaaacagg

SEQ ID NO: 33 - DNA, 253 bp, Artificial Sequence
  Synthetic Polypeptide
tacgtagggtgcaagcgttatccggcattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacagg

SEQ ID NO: 34 - DNA, 253 bp, Artificial Sequence
  Synthetic Polypeptide
tacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacagg

SEQ ID NO: 35 - DNA, 253 bp, Artificial Sequence
  Synthetic Polypeptide
tacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactagaattcccggtgtaacggtggaatgtgtagatatcgggaagaacaccaatggcgaaggcaggtctctgggccgttactgacgctgaggagcgaaagcgtggggagcgaacagg

SEQ ID NO: 36 - DNA, 253 bp, Artificial Sequence
  Synthetic Polypeptide
tacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcaaacagg

SEQ ID NO: 37 - DNA, 253 bp, Artificial Sequence
  Synthetic Polypeptide
tacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacagg

SEQ ID NO: 38 - DNA, 253 bp, Artificial Sequence
  Synthetic Polypeptide
tacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggccgtcactgacgctgaggagcgaaagcgtggggagcgaacagg

SEQ ID NO: 39 - DNA, 253 bp, Artificial Sequence
  Synthetic Polypeptide
tacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacagg

SEQ ID NO: 40 - DNA, 252 bp, Artificial Sequence
  Synthetic Polypeptide
tacgtagggggcgagcgttatccggattcattgggcgtaaagcgcgcgtaggcggcccggcaggcagggggtcaaatggcggggctcaaccccgtcccgccccctgaaccgccgggctcgggtccggtaggggagggtggaacacccggtgtagcggtggaatgcgcagatatcgggtggaacaccggtggcgaaggcggccctctgggccgagaccgacgctgaggcgcgaaagctgggggagcgaacagg

SEQ ID NO: 41 - DNA, 253 bp, Artificial Sequence
  Synthetic Polypeptide
tacgtagggggcgagcgttatccggattcattgggcgtaaagcgcgcgtaggcgtcccttcaagcggcaccgtcgaagccgggggctcaacccccggaagcgggccgaactggggggatcgagtgcggtaggggaaggcggaattcccggtgtagcggtgaaatgcgcagatatcgggaagaacaccgacggcgaaggcagccttctgggccgccactgacgctgaggcgcgaaagctgggggagcgaacagg

SEQ ID NO: 42 - DNA, 253 bp, Artificial Sequence
  Synthetic Polypeptide
tacgtagggggcgagcgttatccggattcattgggcgtaaagcgcgcgtaggcggccgcctaagcggaacctctaatcccggggctcaaccccgcggccgggttccgaactgggcggctcgagtgcggtaggggcaggtggaattcccggtgtagcggtggaatgcgcagatatcgggaggaacaccgatggcgaaggcagcctgctgggccgacactgacgctgaggcgcgaaagccgggggagcgaacagg

SEQ ID NO: 43 - DNA, 253 bp, Artificial Sequence
  Synthetic Polypeptide
tacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggcctactgggcaccaactgacgctgaggctcgaaagtgtgggtagcaaacagg

SEQ ID NO: 44 - DNA, 253 bp, Artificial Sequence
  Synthetic Polypeptide
tacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacagg

SEQ ID NO: 45 - DNA, 253 bp, Artificial Sequence
  Synthetic Polypeptide
tacggaggatccgagcgttatccggatttattgggtttaaagggagcgtaggcggattattaagtcagttgtgaaagtttgcggctcaaccgtaaaattgcagttgatactggtagtcttgagtgcagcagaggtaggcggaattcgtggtgtagcggtgaaatgcttagatatcacgaagaactccgattgcgaaggcagcttactggactgtaactgacgctgatgctcgaaagtgtgggtatcaaacagg

SEQ ID NO: 46 - DNA, 253 bp, Artificial Sequence
  Synthetic Polypeptide
tacggaggatccgagcgttatccggatttattgggtttaaagggagcgtagatgggttgttaagtcagttgtgaaagtttgcggctcaaccgtaaaattgcaattgatactggcagtcttgagtacagttgaggtaggcggaattcgtggtgtagcggtgaaatgcttagatatcacgaagaactccgattgcgaaggcagcttactaacctgtaactgacattgatgctcgaaagtgtgggtatcaaacagg

SEQ ID NO: 47 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacagg

SEQ ID NO: 48 - DNA, 253 bp, Artificial Sequence
  Synthetic Polypeptide
tacggaggatccgagcgttatccggatttattgggtttaaagggagcgtaggcggggtattaagtcagttgtgaaagtttgcggctcaaccgtaaaattgcagttgatactggtatccttgagtgcagcagaggtgggcggaattcgtggtgtagcggtgaaatgcttagatatcacgaagaactccgattgcgaaggcagctcactggagtgtaactgacgctgatgctcgaaagtgtgggtatcaaacagg

SEQ ID NO: 49 - DNA, 253 bp, Artificial Sequence
  Synthetic Polypeptide
tacggaggatccgagcgttatccggatttattgggtttaaagggagcgtagatgggttgttaagtcagttgtgaaagtttgcggctcaaccgtaaaattgcaattgatactggcagtcttgagtacagttgaggtaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 50 - DNA, 253 bp, Artificial Sequence
  Synthetic Polypeptide
tacggaggatccgagcgttatccggatttattgggtttaaagggagcgtaggcggacgcttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 51 - DNA, 253 bp, Artificial Sequence
  Synthetic Polypeptide
tacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 52 - DNA, 253 bp, Artificial Sequence
  Synthetic Polypeptide
tacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 53 - DNA, 253 bp, Artificial Sequence
  Synthetic Polypeptide
tacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacagg

SEQ ID NO: 54 - DNA, 253 bp, Artificial Sequence
  Synthetic Polypeptide
tacggaggatccgagcgttatccggatttattgggtttaaagggagcgtagatgggttgttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 55 - DNA, 253 bp, Artificial Sequence
  Synthetic Polypeptide
tacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcctgctggactgtaactgacgctgatgctcgaaagtgtgggtatcaaacagg

SEQ ID NO: 56 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtagcaaacagg

SEQ ID NO: 57 - DNA, 253 bp, Artificial Sequence
  Synthetic Polypeptide
tacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgttactgacactgatgctcgaaagtgtgggtatcaaacagg

SEQ ID NO: 58 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 59 - DNA, 253 bp, Artificial Sequence
  Synthetic Polypeptide
tacggaggatccgagcgttatccggatttattgggtttaaagggagcgtaggtggattgttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 60 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 61 - DNA, 253 bp, Artificial Sequence
  Synthetic Polypeptide
tacggaggatccgagcgttatccggatttattgggtttaaagggagcgtagatgggttgttaagtcagttgtgaaagtttgcggctcaaccgtaaaattgcaattgatactggcagtcttgagtacagttgaggtaggcggaattcgtggtgtagcggtgaaatgcttagatatcacgaagaactccgattgcgaaggcagcttactaacctgtaactgacattgaggctcgaaagtgtgggtatcaaacagg

SEQ ID NO: 62 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcaattgatactggcagtcttgagtacagttgaggtaggcggaattcgtggtgtagcggtgaaatgcttagatatcacgaagaactccgattgcgaaggcagcttactaacctgtaactgacattgatgctcgaaagtgtgggtatcaaacagg

SEQ ID NO: 63 - DNA, 253 bp, Artificial Sequence
  Synthetic Polypeptide
tacggaggatccgagcgttatccggatttattgggtttaaagggagcgtaggtgggttgttaagtcagttgtgaaagtttgcggctcaaccgtaaaattgcaattgatactggcagtcttgagtacagttgaggtaggcggaattcgtggtgtagcggtgaaatgcttagatatcacgaagaactccgattgcgaaggcagcttactaacctgtaactgacattgatgctcgaaagtgtgggtatcaaacagg

SEQ ID NO: 64 - DNA, 253 bp, Artificial Sequence
  Synthetic Polypeptide
tacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacagg

SEQ ID NO: 65 - DNA, 253 bp, Artificial Sequence
  Synthetic Polypeptide
tacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacagg

SEQ ID NO: 66 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaaaagg

SEQ ID NO: 67 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggcgcgaaagtgtgggtatcaaacagg

SEQ ID NO: 68 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatctcgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 69 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattggggctcgaaagtgtgggtatcaaacagg

SEQ ID NO: 70 - DNA, 253 bp, Artificial Sequence
  Synthetic Polypeptide
tacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagcttactaacctgtaactgacattgatgctcgaaagtgtgggtatcaaacagg

SEQ ID NO: 71 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacagg

SEQ ID NO: 72 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactggcattgaggctcgaaagtgtgggtatcaaacagg

SEQ ID NO: 73 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaagttgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 74 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcagctgacattgaggctcgaaagtgtgggtatcaaacagg

SEQ ID NO: 75 - DNA, 253 bp, Artificial Sequence
  Synthetic Polypeptide
tacgtaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 76 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaggtgtgggtatcaaacagg

SEQ ID NO: 77 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcagacagg

SEQ ID NO: 78 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcccgctaagctgcaactgacattgaggctcgaaagtgtgggtatcaaacagg

SEQ ID NO: 79 - DNA, 253 bp, Artificial Sequence
  Synthetic Polypeptide
tacggaggatccgagcgttatccggatttattgggtttaaagggagcgtagatgggttgttaagtcagttgtgaaagtttgcggctcaaccgtaaaattgcaattgatactggcagtcttgagtacagttgaggtaggcggaattcgtggtgtagcggtgaaatgcttaggtatcacgaagaactccgattgcgaaggcagcttactaacctgtaactgacattgatgctcgaaagtgtgggtatcaaacagg

SEQ ID NO: 80 - DNA, 253 bp, Artificial Sequence
  Synthetic Polypeptide
tacggaggatccgagcgttatccggatttattgggtttaaagggggcgtagatgggttgttaagtcagttgtgaaagtttgcggctcaaccgtaaaattgcaattgatactggcagtcttgagtacagttgaggtaggcggaattcgtggtgtagcggtgaaatgcttagatatcacgaagaactccgattgcgaaggcagcttactaacctgtaactgacattgatgctcgaaagtgtgggtatcaaacagg

SEQ ID NO: 81 - DNA, 253 bp, Artificial Sequence
  Synthetic Polypeptide
tacggaggatacgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 82 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacactgaggctcgaaagtgtgggtatcaaacagg

SEQ ID NO: 83 - DNA, 253 bp, Artificial Sequence
  Synthetic Polypeptide
tacggaggatccgagcgttatccggatttattgggtttaaagggagcgtagatggatgtttaagtcagttgtgaaggtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 84 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttaagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 85 - DNA, 253 bp, Artificial Sequence
  Synthetic Polypeptide
tacggaggatccgagcgttatccggatttattgggtttaaagggagcgtagatgggtgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 86 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcctgctggactgtaactgacgctgatgctcgaaagtgtgggtatcaaacagg

SEQ ID NO: 87 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactacgattgcgaaggcagcctgctaagctgcaactgacattgaggctcgaaagtgtgggtatcaaacagg

SEQ ID NO: 88 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttggggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 89 - DNA, 253 bp, Artificial Sequence
  Synthetic Polypeptide
tacggaggatccgagcgttatccggatttattgggtttaaagggagcgtagatggatgtttaagtcagttgtgaaagtttacggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 90 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcggcctgctaagctgcaactgacattgaggctcgaaagtgtgggtatcaaacagg

SEQ ID NO: 91 - DNA, 253 bp, Artificial Sequence
  Synthetic Polypeptide
tacggaggatccgagcgttatccggatttattgggtttaaagggagcgtaggtggattgttaagtcagttgtgaaagtttgcggctcaaccgtaaaattgcaattgatactggcagtcttgagtacagttgaggtaggcggaattcgtggtgtagcggtgaaatgcttagatatcacgaagaactccgattgcgaaggcagcttactaacctgtaactgacattgatgctcgaaagtgtgggtatcaaacagg

SEQ ID NO: 92 - DNA, 253 bp, Artificial Sequence
  Synthetic Polypeptide
tacggaggatccgagcgttatccggatttattgggtttaaagggagcgtaggcggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacagg

SEQ ID NO: 93 - DNA, 253 bp, Artificial Sequence
  Synthetic Polypeptide
tacggaggatccgagcgttatccggatttattgggtttaaagggagcgtagatgggttgttaagtcagttgtgaaagtttgcggctcaaccgtaaaattgcaattgatactggcagtcttgagtacagttgaggtaggcggaattcgtggtgtagcggtgaaatgcttagatatcacgaagaattccgattgcgaaggcagcttactaacctgtaactgacattgatgctcgaaagtgtgggtatcaaacagg

SEQ ID NO: 94 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttggatatcacgaagaactccgattgcgaaggcagcctgctaagctgcaactgacattgaggctcgaaagtgtgggtatcaaacagg

SEQ ID NO: 95 - DNA, 253 bp, Artificial Sequence
  Synthetic Polypeptide
tacggaggatccgagcgttacccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 96 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttggtactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 97 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagtcgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 98 - DNA, 253 bp, Artificial Sequence
  Synthetic Polypeptide
tacggaggatgcgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 99 - DNA, 253 bp, Artificial Sequence
  Synthetic Polypeptide
tacggaggatccgagcgttatccggatttattgggtttaaagggagcgtagatgggttgttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 100 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaaccccgattgcgaaggcagcctgctaagctgcaactgacattgaggctcgaaagtgtgggtatcaaacagg

SEQ ID NO: 101 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcttgctggactgtaactgacgctgatgctcgaaagtgtgggtatcaaacagg

SEQ ID NO: 102 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcttactaacctgtaactgacattgatgctcgaaagtgtgggtatcaaacagg

SEQ ID NO: 103 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgatcgcgaaggcattgtgccggggcagcactgacgctgaggctcgaaagtgcgggtatcaaacagg

SEQ ID NO: 104 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcttactaaaccataactgacactgaagcacgaaagcgtggggatcaaacagg

SEQ ID NO: 105 - DNA, 253 bp, Artificial Sequence
  Synthetic Polypeptide
tacggaggatgcgagcgttatccggatttattgggtttaaagggtgcgtaggcggcacgccaagtcagcggtgaaatttccgggctcaacccggactgtgccgttgaaactggcgagctagagtgcacaagaggcaggcggaatgcgtggtgtagcggtgaaatgcatagatatcacgcagaaccccgattgcgaaggcagcctgctagggtgcgacagacgctgaggcacgaaagcgtgggtatcgaacagg

SEQ ID NO: 106 - DNA, 253 bp, Artificial Sequence
  Synthetic Polypeptide
tacggaggatgcgagcgttatccggatttattgggtttaaagggtgcgtaggcggcacgccaagtcagcggtgaaatttccgggctcaacccggactgtgccgttgaaactggcgagctagagtgcacaagaggcaggcggaatgcgtggtgtagcggtgaaatgcgtagatatcacgcagaaccccgattgcgaaggcagcctgctagggtgcgacagacgctgaggcacgaaagcgtgggtatcgaacagg

SEQ ID NO: 107 - DNA, 253 bp, Artificial Sequence
  Synthetic Polypeptide
tacggaagatgcgagcgttatccggatttattgggtttaaagggtgcgtaggcggaagaataagtcagcggtgaaatgcttcagctcaactggagaattgccgatgaaactgtttttctagagtataaaagaggtatgcggaatgcgtggtgtagcggtgaaatgcatagatatcacgcagaaccccgattgcgaaggcagcatactgggctataactgacgctgaagcacgaaagcgtgggtatcgaacagg

SEQ ID NO: 108 - DNA, 252 bp, Artificial Sequence
  Synthetic Polypeptide
tacggaggatgcgagcgttatccggatttattgggtttaaagggtgcgtaggcggtttattaagttagtggttaaatatttgagctaaactcaattgtgccattaatactggtaaactggagtacagacgaggtaggcggaataagttaagtagcggtgaaatgcatagatataacttagaactccgatagcgaaggcagcttaccagactgtaactgacgctgatgcacgagagcgtgggtagcgaacagg

SEQ ID NO: 109 - DNA, 253 bp, Artificial Sequence
  Synthetic Polypeptide
tacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccatgactgacgctgatgcacgaaagcgtggggatcaaacagg

SEQ ID NO: 110 - DNA, 253 bp, Artificial Sequence
  Synthetic Polypeptide
tacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcttagatatcacgcagaaccccgattgcgaaggcagcctgccaagccattactgacgctgatgcacgaaagcgtggggatcaaacagg

SEQ ID NO: 111 - DNA, 253 bp, Artificial Sequence
  Synthetic Polypeptide
tacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacagg

SEQ ID NO: 112 - DNA, 253 bp, Artificial Sequence
  Synthetic Polypeptide
tacggaggatccgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacagg

SEQ ID NO: 113 - DNA, 253 bp, Artificial Sequence
  Synthetic Polypeptide
tacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaaccccgattgcgaaggcagcctgctagggtgcgacagacgctgaggcacgaaagcgtgggtatcgaacagg

SEQ ID NO: 114 - DNA, 252 bp, Artificial Sequence
  Synthetic Polypeptide
tacggaagatgcgagcgttatccggatttattgggtttaaagggagcgtaggcgggcttttaagtcagcggtcaaatgtcacggctcaaccgtggccagccgttgaaactgcaagccttgagtctgcacagggcacatggaattcgtggtgtagcggtgaaatgcttagatatcacgaagaactccgatcgcgaaggcattgtgccggggcataactgacgctgaggctcgaaagtgcgggtatcaaacagg

SEQ ID NO: 115 - DNA, 252 bp, Artificial Sequence
  Synthetic Polypeptide
tacggaagatgcgagcgttatccggatttattgggtttaaagggagcgtaggcgggcttttaagtcagcggtcaaatgtcacggctcaaccgtggccagccgttgaaactgtaagccttgagtctgcacagggcacatggaattcgtggtgtagcggtgaaatgcttagatatcacgaagaactccgatcgcgaaggcattgtgccggggcagcactgacgctgaggctcgaaagtgcgggtatcaaacagg

SEQ ID NO: 116 - DNA, 252 bp, Artificial Sequence
  Synthetic Polypeptide
tacggaggatccgagcgttatccggatttattgggtttaaagggagcgtaggcgggcttttaagtcagcggtcaaatgtcacggctcaaccgtggccagccgttgaaactgtaagccttgagtctgcacagggcacatggaattcgtggtgtagcggtgaaatgcttagatatcacgaagaactccgatcgcgaaggcattgtgccggggcagcactgacgctgaggctcgaaagtgcgggtatcaaacagg

SEQ ID NO: 117 - DNA, 252 bp, Artificial Sequence
  Synthetic Polypeptide
tacggaagatgcgagcgttatccggatttattgggtttaaagggagcgtaggcgggcttttaagtcagcggtcaaatgtcacggctcaaccgtggccagccgttgaaactgtaagccttgagtctgcacagggcacatggaattcgtggtgtagcggtgaaatgcttagatatcacgaagaactccgatcgcgaaggcattgtgccggggcagcactgacgctgaggctcgaaagtgtgggtatcaaacagg

SEQ ID NO: 118 - DNA, 252 bp, Artificial Sequence
  Synthetic Polypeptide
tacggaggatgcgagcgttatccggatttattgggtttaaagggagcgtaggcgggcttttaagtcagcggtcaaatgtcacggctcaaccgtggccagccgttgaaactgtaagccttgagtctgcacagggcacatggaattcgtggtgtagcggtgaaatgcttagatatcacgaagaactccgatcgcgaaggcattgtgccggggcagcactgacgctgaggctcgaaagtgcgggtatcaaacagg

SEQ ID NO: 119 - DNA, 252 bp, Artificial Sequence
  Synthetic Polypeptide
tacggaagatgcgagcgttatccggatttattgggtttaaagggagcgtaggcgggcttttaagtcagcggtcaaatgtcacggctcaaccgtggccagccgttgaaactgtaagccttgagtctgcacagggcacatggaattcgtggtgtagcggtgaaatgcttagatatcacgaagaactccgatcgcgaaggcattgtgccggggcataactgacgctgaggctcgaaagtgcgggtatcaaacagg

SEQ ID NO: 120 - DNA, 252 bp, Artificial Sequence
  Synthetic Polypeptide
tacggaagatgcgagcgttatccggatttattgggtttaaagggagcgtaggcgggcttttaagtcagcggtcaaatgtcacggctcaaccgtggccagccgttgaaactgtaagccttgagtctgcacagggcacatggaattcgtggtgtagcggtgaaatgcttagatattacgaagaactccgatcgcgaaggcattgtgccggggcagcactgacgctgaggctcgaaagtgcgggtatcaaacagg

SEQ ID NO: 121 - DNA, 252 bp, Artificial Sequence
  Synthetic Polypeptide
tacggaagatgcgagcgttatccggatttattgggtttaaagggagcgtaggcgggcctttaagtcagcggtcaaatgtcacggctcaaccgtggccagccgttgaaactgtaagccttgagtctgcacagggcacatggaattcgtggtgtagcggtgaaatgcttagatatcacgaagaactccgatcgcgaaggcattgtgccggggcagcactgacgctgaggctcgaaagtgcgggtatcaaacagg

SEQ ID NO: 122 - DNA, 252 bp, Artificial Sequence
  Synthetic Polypeptide
tacggaagatgcgagcgttatccggatttattgggtttaaagggagcgtaggcgggcttttaagtcagcggtcaaatgtcacggctcaaccgtggccagccgttgaaactgtaagccttgagtctgcacagggcacatggaattcgtggtgtagcggtgaaatgcttagatatcacgaagaactccgatcgcgaaggcattgtgccggggcagcactgacgctgaggctcgaaagtgcgggtatcaagcagg

SEQ ID NO: 123 - DNA, 252 bp, Artificial Sequence
  Synthetic Polypeptide
tacggaagatgcgagcgttatccggatttattgggtttaaagggagcgtaggcgggcttttaagtcagcggtaaaatgtcacggctcaaccgtggccagccgttgaaactgtaagccttgagtctgcacagggcacatggaattcgtggtgtagcggtgaaatgcttagatatcacgaagaactccgatcgcgaaggcattgtgccggggcagcactgacgctgaggctcgaaagtgcgggtatcaaacagg

SEQ ID NO: 124 - DNA, 253 bp, Artificial Sequence
  Synthetic Polypeptide
tacggaggatccaagcgttatccggatttattgggtttaaagggtgcgtaggcggtttgataagttagaggtgaaataccggtgcttaacaccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacagg

SEQ ID NO: 125 - DNA, 253 bp, Artificial Sequence
  Synthetic Polypeptide
tacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacagg

SEQ ID NO: 126 - DNA, 253 bp, Artificial Sequence
  Synthetic Polypeptide
tacggaggatccaagcgttatccggatttattgggtttaaagggtgcgtaggcggtttgataagttagaggtgaaataccggggctcaactccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacagg

SEQ ID NO: 127 - DNA, 253 bp, Artificial Sequence
  Synthetic Polypeptide
tacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatggtgtagcggtgaaatgcttagagatcatacagaacaccgattgcgaaggcagcttaccaaactatatctgacgttgaggcacgaaagcgtggggagcaaacagg

SEQ ID NO: 128 - DNA, 253 bp, Artificial Sequence
  Synthetic Polypeptide
tacggaggatccgagcgttatccggatttattgggtttaaagggtgcgtaggctgttttttaagttagaggtgaaagctcgacgctcaacgtcgaaattgcctctgatactgagagactagagtgtagttgcggaaggcggaatgtgtggtgtagcggtgaaatgcttagatatcacacagaacaccgattgcgaaggcagctttccaagctattactgacgctgaggcacgaaagcgtggggagcgaacagg

SEQ ID NO: 129 - DNA, 253 bp, Artificial Sequence
  Synthetic Polypeptide
tacggaggatccgagcgttatccggatttattgggtttaaagggtgcgtaggcggtttgataagttagaggtgaaagctcgatgctcaacatcgaaattgcctctgatactgtcagactagagtgtagttgcggaaggcggaatgtgtggtgtagcggtgaaatgcttagatatcacacagaacaccgattgcgaaggcagctttccaagctattactgacgctgaggcacgaaagcgtgggtagcgaacagg

SEQ ID NO: 130 - DNA, 252 bp, Artificial Sequence
  Synthetic Polypeptide
tacggaagatgcgagcgttatccggatttattgggtttaaagggagcgtaggcgggcttttaagtcagcggtcaaatgtcacggctcaaccgtggccagccgttgaaactgtaagccttgagtctgcacagggcacatggaattcgtggtgtagcggtgaaatgcttagatatcacgaagaactccgattgcgaaggcagcctgctaagctgcaactgacattgaggctcgaaagtgtgggtatcaaacagg

SEQ ID NO: 131 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgatcgcgaaggcattgtgccggggcagcactgacgctgaggctcgaaagtgcgggtatcaaacagg

SEQ ID NO: 132 - DNA, 253 bp, Artificial Sequence
  Synthetic Polypeptide
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagctcactggagtgtaactgacgctgatgctcgaaagtgtgggtatcaaacagg

SEQ ID NO: 133 - DNA, 252 bp, Artificial Sequence
  Synthetic Polypeptide
tacggaggatgcgagcgttatccggatttattgggtttaaagggtgcgtaggcggcctgttaagtcagcggtaaaagcccggggctcaaccccggcgagccgttgaaactggcgggctcgagttggcgagaagtatgcggaatgcgcggtgtagcggtgaaatgcttagatatcgcgcagaactccgattgcgaaggcagcataccggtgccagactgacgctgaggcacgaaagcgtgggtatcgaacagg

SEQ ID NO: 134 - DNA, 252 bp, Artificial Sequence
  Synthetic Polypeptide
tacggaggatgcgagcgttatccggatttattgggtttaaagggtgcgtaggcggcccgttaagtcagcggtaaaagcccggggctcaaccccggcgagccgttgaaactggcgggcttgagttggcgagaagtatgcggaatgcgcggtgtagcggtgaaatgcttagatatcgcgcagaactccgattgcgaaggcagcataccggtgccacactgacgctgaggcacgaaagcgtgggtatcgaacagg

SEQ ID NO: 135 - DNA, 253 bp, Artificial Sequence
  Synthetic Polypeptide
tacgtaggtggcgagcgttgtccggatttattgggcgtaaagggaacgcaggcggtcttttaagtctgatgtgaaagccttcggcttaaccgaagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacagg

SEQ ID NO: 136 - DNA, 252 bp, Artificial Sequence
  Synthetic Polypeptide
tacgtaggtcccgagcgttgtccggatttattgggcgtaaagcgagcgcaggcggtttgataagtctgaagttaaaggctgtggctcaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcgaacagg

SEQ ID NO: 137 - DNA, 253 bp, Artificial Sequence
  Synthetic Polypeptide
tacgtatggagcaagcgttatccggatttactgggtgtaaagggagtgtaggtggcatcacaagtcagaagtgaaagcccggggctcaaccccgggactgcttttgaaactgtggagctggagtgcaggagaggcaagtggaattcctagtgtagcggtgaaatgcgtagatattaggaggaacaccagtggcgaaggcggcttgctggactgtaactgacactgaggctcgaaagcgtggggagcaaacagg

SEQ ID NO: 138 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccgggactgcttttgaaactgcagggctagagtgcaggaggggcaagtggaattcctagtgtagcggtgaaatgcgtagatattaggaggaacaccagtggcgaaggcggcttgctggactgtaactgacactgaggctcgaaagcgtggggagcaaacagg

SEQ ID NO: 139 - DNA, 253 bp, Artificial Sequence
  Synthetic Polypeptide
tacgtaggtggcaagcgttgtccggaattactgggtgtaaagggagcgcaggcgggtgatcaagtcagctgtgaaaactacgggcttaacccgtagactgcagttgaaactgctcatcttgagtgaagtagaggttggcggaattccgagtgtagcggtgaaatgcgtagatattcggaggaacaccggtggcgaaggcggccaactgggctttaactgacgctgaggctcgaaagtgtggggagcaaacagg

SEQ ID NO: 140 - DNA, 253 bp, Artificial Sequence
  Synthetic Polypeptide
tacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 141 - DNA, 253 bp, Artificial Sequence
  Synthetic Polypeptide
tacgtagggggcaagcgttatccggatttactgggtgtaaagggagcgtagacggatggacaagtctgatgtgaaaggctggggctcaaccccgggactgcattggaaactgcccgtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 142 - DNA, 253 bp, Artificial Sequence
  Synthetic Polypeptide
tacgtagggggcaagcgttatccggatttactgggtgtaaagggagcgtagacggcgcagcaagtctgatgtgaaaggcaggggcttaacccctggactgcattggaaactgttgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 143 - DNA, 253 bp, Artificial Sequence
  Synthetic Polypeptide
tacgtagggggcaagcgttatccggatttactgggtgtaaagggagcgtagacggactggcaagtctgatgtgaaagacgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 144 - DNA, 253 bp, Artificial Sequence
  Synthetic Polypeptide
tacgtagggggcaagcgttatccggatttactgggtgtaaagggagcgtaggcggcggagcaagtcagaagtgaaagcccggggctcaaccccgggacggcttttgaaactgccctgcttgatttcaggagaggtaagcggaattcctagtgtagcggtgaaatgcgtagatattaggaggaacaccagtggcgaaggcggcttactggactgacaatgacgctgaggctcgaaagcgtggggagcaaacagg

SEQ ID NO: 145 - DNA, 253 bp, Artificial Sequence
  Synthetic Polypeptide
tacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacagg

SEQ ID NO: 146 - DNA, 253 bp, Artificial Sequence
  Synthetic Polypeptide
tacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgataactgacgctgaggctcgaaagcgtggggagcaaacagg

SEQ ID NO: 147 - DNA, 253 bp, Artificial Sequence
  Synthetic Polypeptide
tacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggcttactggacggtaactgacgttgaggctcgaaagcgtggggagcaaacagg

SEQ ID NO: 148 - DNA, 253 bp, Artificial Sequence
  Synthetic Polypeptide
tacgtatggagcaagcgttatccggatttactgggtgtaaagggtgcgtaggtggcagtgcaagtcagatgtgaaaggccggggctcaaccccggagctgcatttgaaactgcatagctggagtacaggagaggcaggcggaattcctagtgtagcggtgaaatgcgtagatattaggaggaacaccagtggcgaaggcggcctgctggactgttactgacactgaggcacgaaagcgtggggagcaaacagg

SEQ ID NO: 149 - DNA, 253 bp, Artificial Sequence
  Synthetic Polypeptide
tacgtatggagcaagcgttatccggatttactgggtgtaaagggtgcgtaggtggcagtgcaagtcagatgtgaaaggccggggctcaaccccggagctgcatttgaaactgctcggctagagtacaggagaggcaggcggaattcctagtgtagcggtgaaatgcgtagatattaggaggaacaccagtggcgaaggcggcctgctggactgttactgacactgaggcacgaaagcgtggggagcaaacagg

SEQ ID NO: 150 - DNA, 253 bp, Artificial Sequence
  Synthetic Polypeptide
tacgtatggtgcaagcgttatccggatttactgggtgtaaagggagcgcaggcggtacggcaagtctgatgtgaaagcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacagg

SEQ ID NO: 151 - DNA, 253 bp, Artificial Sequence
  Synthetic Polypeptide
tacgtatggtgcaagcgttatccggatttactgggtgtaaagggagcgcaggcggtacggcaagtctgatgtgaaagcccggggctcaaccccggtactgcattggaaactgtcggactagagtgtcggaggggtaagtggaattcctagtgtagcggtgaaatgcgtagatattaggaggaacaccagtggcgaaggcggcttactggacgataactgacgctgaggctcgaaagcgtggggagcaaacagg

SEQ ID NO: 152 - DNA, 253 bp, Artificial Sequence
  Synthetic Polypeptide
tacgtatggtgcaagcgttatccggatttactgggtgtaaagggagcgcaggcggaaggctaagtctgatgtgaaagcccggggctcaaccccggtactgcattggaaactggtcatctagagtgtcggaggggtaagtggaattcctagtgtagcggtgaaatgcgtagatattaggaggaacaccagtggcgaaggcggcttactggacgataactgacgctgaggctcgaaagcgtggggagcaaacagg

SEQ ID NO: 153 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 154 - DNA, 253 bp, Artificial Sequence
  Synthetic Polypeptide
tacgtagggggcaagcgttatccggatttactgggtgtaaagggagcgtagacggattagcaagtctgatgtgaaaggcaggggctcaacccctggactgcattggaaactgccagtcttgagtgccggagaggtaagcggaattcctagtgtagcggtgaaatgcgtagatattaggaggaacaccagtggcgaaggcggcttactggacggcaactgacgttgaggctcgaaagcgtggggagcaaacagg

SEQ ID NO: 155 - DNA, 253 bp, Artificial Sequence
  Synthetic Polypeptide
tacgtatggtgcaagcgttatccggatttactgggtgtaaagggagcgcaggcggtacggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 156 - DNA, 253 bp, Artificial Sequence
  Synthetic Polypeptide
tacgtagggggcgagcgttatccggatttactgggtgtaaagggagcgtagacggcgtatcaagtctgatgtgaaaggcaggggcttaacccctggactgcattggaaactggtatgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 157 - DNA, 253 bp, Artificial Sequence
  Synthetic Polypeptide
tacgtatggtgcaagcgttatccggatttactgggtgtaaagggggcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 158 - DNA, 253 bp, Artificial Sequence
  Synthetic Polypeptide
tacgtatggtgcaagcgttatccggatttactgggtgtaaagggagcgcaggcggtgcggcaagtctgatgtggaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 159 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagggtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 160 - DNA, 253 bp, Artificial Sequence
  Synthetic Polypeptide
tacgtagggggcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 161 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtacttgagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 162 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcagaattcctagtgtagcggtgaaatgcgtagatattaggaggaacaccagtggcgaaggcggcttactggacgataactgacgctgaggctcgaaagcgtggggagcaaacagg

SEQ ID NO: 163 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggcactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 164 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcctgctggactgttactgacactgaggcacgaaagcgtggggagcaaacagg

SEQ ID NO: 165 - DNA, 253 bp, Artificial Sequence
  Synthetic Polypeptide
tacgtatggtgcaagcgttatccggatttactgggtgtagagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 166 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcgacttactggacgataactgacgctgaggctcgaaagcgtggggagcaaacagg

SEQ ID NO: 167 - DNA, 253 bp, Artificial Sequence
  Synthetic Polypeptide
tacgtatggtgcaagcgttatccggatttactgggtgtaaagggagcgcaggcggtgcggcaagcctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 168 - DNA, 253 bp, Artificial Sequence
  Synthetic Polypeptide
tacgtagggggcaagcgttatccggatttactgggtgtaaagggagcgtagacggctgtgcaagtctgaagtgaaaggcatgggctcaacctgtggactgctttggaaactgtgcagctagagtgtcggagaggtaagtggaattcctagtgtagcggtgaaatgcgtagatattaggaggaacaccagtggcgaaggcggcttactggacgataactgacgctgaggctcgaaagcgtggggagcaaacagg

SEQ ID NO: 169 - DNA, 253 bp, Artificial Sequence
  Synthetic Polypeptide
tacgtatggtgcaagcgttatccggatttactgggtgtaaagggagcgcaggcggtgcggcaagtctgatgtgaaggcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 170 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcagaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 171 - DNA, 253 bp, Artificial Sequence
  Synthetic Polypeptide
aacgtagggtgcaagcgttgtccggaattactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 172 - DNA, 253 bp, Artificial Sequence
  Synthetic Polypeptide
tacgtatggggcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 173 - DNA, 253 bp, Artificial Sequence
  Synthetic Polypeptide
tacgtagggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 174 - DNA, 253 bp, Artificial Sequence
  Synthetic Polypeptide
tacgtatggtgcaagcgttatccggatttactgggtgtaaagggagcgcaggcggtgcggtaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 175 - DNA, 253 bp, Artificial Sequence
  Synthetic Polypeptide
tacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggctttctggactgtaactgacgctgaggctcgaaagctagggtagcgaacggg

SEQ ID NO: 176 - DNA, 253 bp, Artificial Sequence
  Synthetic Polypeptide
tacgtagggggcaagcgttatccggatttactgggtgtaaagggagcgtagacggatggacaagtctgatgtgaaaggctggggctcaaccccggggctgcattggaaactgcccgtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 177 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagagattaggaggaacaccagtggcgaaggcggcttactggacgataactgacgctgaggctcgaaagcgtggggagcaaacagg

SEQ ID NO: 178 - DNA, 253 bp, Artificial Sequence
  Synthetic Polypeptide
aacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 179 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacggtaactgacgctgaggctcgaaagcgtggggagcaaacagg

SEQ ID NO: 180 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggcgcgaaagcgtggggagcaaacagg

SEQ ID NO: 181 - DNA, 253 bp, Artificial Sequence
  Synthetic Polypeptide
tacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 182 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgagatgcgtagatattaggaggaacaccagtggcgaaggcggcttactggacgataactgacgctgaggctcgaaagcgtggggagcaaacagg

SEQ ID NO: 183 - DNA, 253 bp, Artificial Sequence
  Synthetic Polypeptide
tacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaagaacaccagtggcgaaggcggctttctggactgtaactgacgctgaggctcgaaagctagggtagcgaacggg

SEQ ID NO: 184 - DNA, 253 bp, Artificial Sequence
  Synthetic Polypeptide
tacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgttgaggctcgaaagcgtggggagcaaacagg

SEQ ID NO: 185 - DNA, 253 bp, Artificial Sequence
  Synthetic Polypeptide
tacgcatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 186 - DNA, 253 bp, Artificial Sequence
  Synthetic Polypeptide
tacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacagg

SEQ ID NO: 187 - DNA, 253 bp, Artificial Sequence
  Synthetic Polypeptide
tacgtatggtgcaagcgttatccggatttactgggtgtaaagggagcgcaggcggtgcggcaagtctgatgtgaaagccaggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 188 - DNA, 253 bp, Artificial Sequence
  Synthetic Polypeptide
aacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggtctactgggcaccaactgacgctgaggctcgaaagcatgggtagcaaacagg

SEQ ID NO: 189 - DNA, 253 bp, Artificial Sequence
  Synthetic Polypeptide
aacgtaggtcacaagcgttgtccggaattactgggtgtaaagggagcgcaggcgggagaacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 190 - DNA, 253 bp, Artificial Sequence
  Synthetic Polypeptide
aacgtaggtcacaagcgttgtccggaattactgggtgtaaagggagcgcaggcgggagaacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagcgtggggagcaaacagg

SEQ ID NO: 191 - DNA, 253 bp, Artificial Sequence
  Synthetic Polypeptide
aacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggagtgcgtagatatcgggaggaacaccagtggcgaaggcggcctactgggcaccaactgacgctgaggctcgaaagtgtgggtagcaaacagg

SEQ ID NO: 192 - DNA, 253 bp, Artificial Sequence
  Synthetic Polypeptide
aacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagcgtggggagcaaacagg

SEQ ID NO: 193 - DNA, 253 bp, Artificial Sequence
  Synthetic Polypeptide
aacgtagggtgcaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 194 - DNA, 253 bp, Artificial Sequence
  Synthetic Polypeptide
aacgtaggtcacaagcgttgtccggaattactgggtgtaaagggagcgcaggcgggaagacaagttggaagtgaaatccatgggctcaacccatgaactgctttcaaaactgtttttcttgagtagtgcaggggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 195 - DNA, 253 bp, Artificial Sequence
  Synthetic Polypeptide
aacgtaggtcacgagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 196 - DNA, 253 bp, Artificial Sequence
  Synthetic Polypeptide
tacgtagggagcaagcgttgtccggatttactgggtgtaaagggtgcgtaggcggattggcaagtcagaagtgaaatccatgggcttaacccatgaactgcttttgaaactgttagtcttgagtgaagtagaggtaggcggaattcccggtgtagcggtgaaatgcgtagagatcgggaggaacaccagtggcgaaggcggcctactgggctttaactgacgctgaggcacgaaagtgtgggtagcaaacagg

SEQ ID NO: 197 - DNA, 253 bp, Artificial Sequence
  Synthetic Polypeptide
aacgtagggtgcaagcgttgtccggaattactgggtgtaaagggagcgcaggcgggaagacaagttggaagtgaaaaccatgggctcaacccatgaattgctttcaaaactgtttttcttgagtagtgcagaggtagatggaattcccggtgtagcggtggaatgcgtagatatcgggaggaacaccagtggcgaaggcggtctactgggcaccaactgacgctgaggctcgaaagtgtgggtagcaaacagg

SEQ ID NO: 198 - DNA, 253 bp, Artificial Sequence
  Synthetic Polypeptide
tacgtagggagcgagcgttgtccggaattactgggtgtaaagggagcgtaggcgggaaagcaagttggaagtgaaatgcatgggcttaacccatgagctgctttcaaaactgtttttcttgagtgaagtagaggcaggcggaattcctagtgtagcggtgaaatgcgtagatattaggaggaacaccagtggcgaaggcggcctgctgggctttaactgacgctgaggctcgaaagcgtgggtagcaaacagg

SEQ ID NO: 199 - DNA, 253 bp, Artificial Sequence
  Synthetic Polypeptide
aacgtaggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcggcttactggacgataactgacgctgaggctcgaaagcgtggggagcaaacagg

SEQ ID NO: 200 - DNA, 253 bp, Artificial Sequence
  Synthetic Polypeptide
aacgtagggtgcaagcgttgtccggaattactgggtgtaaagggagcgcaggcggaccggcaagttggaagtgaaaactatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacagg

SEQ ID NO: 201 - DNA, 253 bp, Artificial Sequence
  Synthetic Polypeptide
aacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcttactggacgataactgacgctgaggctcgaaagcgtggggagcaaacagg

SEQ ID NO: 202 - DNA, 253 bp, Artificial Sequence
  Synthetic Polypeptide
aacgtaggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggagtgcgtagatatcgggaggaacaccagtggcgaaggcggcctactgggcaccaactgacgctgaggctcgaaagtgtgggtagcaaacagg

SEQ ID NO: 203 - DNA, 253 bp, Artificial Sequence
  Synthetic Polypeptide
aacgtaggtcccaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 204 - DNA, 253 bp, Artificial Sequence
  Synthetic Polypeptide
aacgtaggtcacaagcgttgtccggaattactgggtgtaaagggagcgcaggcggaccggcaagttggaagtgaaaactatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacagg

SEQ ID NO: 205 - DNA, 253 bp, Artificial Sequence
  Synthetic Polypeptide
aacgtaggtcacaagcgttgtccggaattactgggtgtaaagggagcgcaggcgggaagacaagttggaagtgaaatctatgggctcaacccataaactgctttcaaaactgtttttcttgagtagtgcagaggtagatggaattcccggtgtagcggtggaatgcgtagatatcgggaggaacaccagtggcgaaggcggtctactgggcaccaactgacgctgaggctcgaaagcatgggtagcaaacagg

SEQ ID NO: 206 - DNA, 253 bp, Artificial Sequence
  Synthetic Polypeptide
tacgtagggagcgagcgttgtccggaattactgggtgtaaagggagcgtaggcgggatcgcaagtcagatgtgaaaactatgggcttaacccataaactgcatttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacagg

SEQ ID NO: 207 - DNA, 253 bp, Artificial Sequence
  Synthetic Polypeptide
aacgtaggtcacaagcgttgtccggaattactggatgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 208 - DNA, 253 bp, Artificial Sequence
  Synthetic Polypeptide
aacgtagggtgcaagcgttgtccggaattactgggtgtaaagggagcgcaggcgggaagacaagttggaagtgaaaaccatgggctcaacccatgaattgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 209 - DNA, 253 bp, Artificial Sequence
  Synthetic Polypeptide
tacgtaggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 210 - DNA, 253 bp, Artificial Sequence
  Synthetic Polypeptide
tacgtaggtggcaagcgttgtccggatttactgggtgtaaagggcgtgtagccggggagacaagtcagatgtgaaatctggaggctcaacctccaaactgcatttgaaactgtttctcttgagtatcggagaggtaatcggaattcctagtgtagcggtgaaatgcgtagatattaggaagaacaccagtggcgaaggcggattactggacgacaactgacggtgaggcgcgaaagcgtggggagcaaacagg

SEQ ID NO: 211 - DNA, 253 bp, Artificial Sequence
  Synthetic Polypeptide
tacgtaggtggcaagcgttgtccggatttactgggtgtaaagggcgtgcagccgggcatgcaagtcagatgtgaaatctcagggcttaaccctgaaactgcatttgaaactgtatgtcttgagtgccggagaggtaatcggaattccttgtgtagcggtgaaatgcgtagatataaggaagaacaccagtggcgaaggcggattactggacggtaactgacggtgaggcgcgaaagcgtggggagcgaacagg

SEQ ID NO: 212 - DNA, 253 bp, Artificial Sequence
  Synthetic Polypeptide
tacgtatggagcaagcgttatccggatttactgggtgtaaagggagcgtaggcggtcctgcaagtctgatgtgaaaggccggggctcaaccccgggactgcattggaaactgtaggactagagtgtcggaggggtaagtggaattcctagtgtagcggtgaaatgcgtagatattaggaggaacaccagtggcgaaggcggcttactggacgactactgacgctgaggctcgaaagcgtggggagcaaacagg

SEQ ID NO: 213 - DNA, 253 bp, Artificial Sequence
  Synthetic Polypeptide
tacgtaggtggcaagcgttatccggatttactgggtgtaaagggcgtgtaggcgggattgcaagtcagatgtgaaaaccacgggctcaacctgtggcctgcatttgaaactgtagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacagg

SEQ ID NO: 214 - DNA, 253 bp, Artificial Sequence
  Synthetic Polypeptide
tacgtagggagcgagcgttatccggatttactgggtgtaaagggcgtgtaggcggggaagcaagtcagatgtgaaaaccagtggctcaaccactggcctgcatttgaaactgtttttcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacagg

SEQ ID NO: 215 - DNA, 253 bp, Artificial Sequence
  Synthetic Polypeptide
tacgtagggggcaagcgttatccggatttactgggtgtaaagggagcgtagacggagaggcaagtctgatgtgaaaacccggggctcaaccccgggactgcattggaaactgtttttctagagtgtcggagaggtaagcggaattcctagtgtagcggtgaaatgcgtagatattaggaggaacaccagtggcgaaggcggcttactggacgatgactgacgttgaggctcgaaagcgtggggagcaaacagg

SEQ ID NO: 216 - DNA, 253 bp, Artificial Sequence
  Synthetic Polypeptide
tacgtagggggcaagcgttatccggatttactgggtgtaaagggagcgtagacggcgaggcaagtctgatgtgaaagcctggggcttaaccccggaactgcattggaaactgctttgctggagtgccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 217 - DNA, 253 bp, Artificial Sequence
  Synthetic Polypeptide
tacgtagggggcaagcgttatccggatttactgggtgtaaagggagcgtagacggcgaggcaagtctgatgtgaaaacccggggctcaaccccgtgactgcattggaaactgttttgcttgagtgccggagaggtaagcggaattcctagtgtagcggtgaaatgcgtagatattaggaggaacaccagtggcgaaggcggcttactggacggcaactgacgttgaggctcgaaagcgtggggagcaaacagg

SEQ ID NO: 218 - DNA, 253 bp, Artificial Sequence
  Synthetic Polypeptide
tacgtagggggcaagcgttatccggatttactgggtgtaaagggagcgtagacggctttgcaagtctgacgtgaaactccggggctcaactccggaactgcgttggaaactgtaaggcttgagtgccggagaggtaagcggaattcctagtgtagcggtgaaatgcgtagatattaggaggaacaccagtggcgaaggcggcttactggacggcaactgacgttgaggctcgaaagcgtggggagcaaacagg

SEQ ID NO: 219 - DNA, 253 bp, Artificial Sequence
  Synthetic Polypeptide
tacgtagggggcaagcgttatccggatttactgggtgtaaagggagcgtagacggcaaggcaagtctgaagtgaaagcccggtgcttaacgccgggactgctttggaaactgtttagctggagtgccggagaggtaagcggaattcctagtgtagcggtgaaatgcgtagatattaggaagaacaccagtggcgaaggcggcttactggacggtaactgacgttgaggctcgaaagcgtggggagcaaacagg

SEQ ID NO: 220 - DNA, 253 bp, Artificial Sequence
  Synthetic Polypeptide
tacgtagggggcaagcgttatccggatttactgggtgtaaagggagcgtagacggcgagacaagtctgaagtgaaagcccggggctcaaccccgggactgctttggaaactgccttgctagagtgctggagaggtaagtggaattcctagtgtagcggtgaaatgcgtagatattaggaggaacaccagtggcgaaggcggcttactggacagtaactgacgttgaggctcgaaagcgtggggagcaaacagg

SEQ ID NO: 221 - DNA, 253 bp, Artificial Sequence
  Synthetic Polypeptide
tacgtagggggcaagcgttatccggatttactgggtgtaaagggagcgtagacggcaaggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 222 - DNA, 253 bp, Artificial Sequence
  Synthetic Polypeptide
tacgtagggggcaagcgttatccggatttactgggtgtaaagggagcgtagacggatggacaagtctgatgtgaaaggctggggctcaaccccgggactgcattggaaactgcccgtcttgagtgccggagaggtaagcggaattcctagtgtagcggtgaaatgcgtagatattgggaagaacaccagtggcgaaggcggctttctggactgtaactgacgctgaggctcgaaagctagggtagcgaacggg

SEQ ID NO: 223 - DNA, 253 bp, Artificial Sequence
  Synthetic Polypeptide
tacgtagggagcaagcgttatccggatttactgggtgtaaagggcgcgcaggcgggccggtaagttggaagtgaaatctatgggcttaacccataaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacagg

SEQ ID NO: 224 - DNA, 253 bp, Artificial Sequence
  Synthetic Polypeptide
tacgtagggggcaagcgttatccggatttactgggtgtaaagggagcgtagacggaacagcaagtctgatgtgaaaacccggggctcaaccccgggactgcattggaaactgttgatctagagtgtcggagaggtaagtggaattcctagtgtagcggtgaaatgcgtagatattaggaggaacaccagtggcgaaggcggcttactggacgatgactgacgttgaggctcgaaagcgtggggagcaaacagg

SEQ ID NO: 225 - DNA, 253 bp, Artificial Sequence
  Synthetic Polypeptide
tacgtagggggcaagcgttatccggatttactgggtgtaaagggagcgcagacggcgatgcaagtctggagtgaaagcccggggctcaaccccgggactgctttggaaactgtatggctagagtgctggagaggcaagcggaattcctagtgtagcggtgaaatgcgtagatattaggaagaacaccagtggcgaaggcggcttgctggacagtaactgacgttcaggctcgaaagcgtggggagcaaacagg

SEQ ID NO: 226 - DNA, 253 bp, Artificial Sequence
  Synthetic Polypeptide
tacgtatggagcaagcgttatccggatttactgggtgtaaagggagtgtaggtggccatgcaagtcagaagtgaaaatccggggctcaaccccggaactgcttttgaaactgtaaggctagagtgcaggaggggtgagtggaattcctagtgtagcggtgaaatgcgtagatattaggaggaacaccagtggcgaaggcggctcactggactgtaactgacactgaggctcgaaagcgtggggagcaaacagg

SEQ ID NO: 227 - DNA, 253 bp, Artificial Sequence
  Synthetic Polypeptide
tacgtaggtggcgagcgttatccggatttactgggtgtaaagggcgcgtaggcgggaatgcaagtcagatgtgaaatccaagggctcaacccttgaactgcatttgaaactgtatttcttgagtgtcggagaggttgacggaattcctagtgtagcggtgaaatgcgtagatattaggaggaacaccagtggcgaaggcggtcaactggacgataactgacgctgaggcgcgaaagcgtggggagcaaacagg

SEQ ID NO: 228 - DNA, 253 bp, Artificial Sequence
  Synthetic Polypeptide
tacgtagggggcaagcgttatccggatttactgggtgtaaagggagcgcagacggcactgcaagtctgaagtgaaagcccggggctcaaccccgggactgctttggaaactgtagagctagagtgctggagaggcaagcggaattcctagtgtagcggtgaaatgcgtagatattaggaagaacaccagtggcgaaggcggcttgctggacagtaactgacgttcaggctcgaaagcgtggggagcaaacagg

SEQ ID NO: 229 - DNA, 253 bp, Artificial Sequence
  Synthetic Polypeptide
tacgtaggtggcaagcgttgtccggatttactgggtgtaaagggcgtgtagccgggaaggcaagtcagatgtgaaatccacgggctcaactcgtgaactgcatttgaaactgtttttcttgagtatcggagaggcaatcggaattcctagtgtagcggtgaaatgcgtagatattaggaggaacaccagtggcgaaggcggattgctggacgacaactgacggtgaggcgcgaaagcgtggggagcaaacagg

SEQ ID NO: 230 - DNA, 253 bp, Artificial Sequence
  Synthetic Polypeptide
tacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacagg

SEQ ID NO: 231 - DNA, 253 bp, Artificial Sequence
  Synthetic Polypeptide
tacgtagggggcaagcgttatccggatttactgggtgtaaagggagcgtagacggtaaagcaagtctgaagtgaaagcccggggctcaaccccgggactgctttggaaactgtttaactagagtgctggagaggtaagcggaattcctagtgtagcggtgaaatgcgtagatattaggaggaacaccagtggcgaaggcggcttactggacagtaactgacgttgaggctcgaaagcgtggggagcaaacagg

SEQ ID NO: 232 - DNA, 253 bp, Artificial Sequence
  Synthetic Polypeptide
tacgtagggggcaagcgttatccggatttactgggtgtaaagggtgcgtaggtggcaaggcaagtcagatgtgaaagcccggggctcaaccccggtactgcatttgaaactgtctagctagagtgcaggagaggtaagcggaattcctagtgtagcggtgaaatgcgtagatattaggaggaacaccagtggcgaaggcggcttactggactgtaactgacactgaggcacgaaagcgtggggagcaaacagg

SEQ ID NO: 233 - DNA, 253 bp, Artificial Sequence
  Synthetic Polypeptide
tacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 234 - DNA, 253 bp, Artificial Sequence
  Synthetic Polypeptide
tacgtagggggcaagcgttatccggatttactgggtgtaaagggagcgcagacggcactgcaagtctgaagtgaaagcccggggctcaaccccgggactgctttggaaactgtagggctagagtgctggagaggcaagcggaattcctagtgtagcggtgaaatgcgtagatattaggaagaacaccagtggcgaaggcggcttgctggacagtaactgacgttcaggctcgaaagcgtggggagcaaacagg

SEQ ID NO: 235 - DNA, 253 bp, Artificial Sequence
  Synthetic Polypeptide
tacgtagggggcaagcgttatccggatttactgggtgtaaagggagcgtagacggcgacgcaagtctgaagtgaaatacccgggctcaacctgggaactgctttggaaactgtgttgctagagtgctggagaggtaagcggaattcctagtgtagcggtgaaatgcgtagatattaggaagaacaccagtggcgaaggcggcttactggacagtaactgacgttgaggctcgaaagcgtggggagcaaacagg

SEQ ID NO: 236 - DNA, 253 bp, Artificial Sequence
  Synthetic Polypeptide
tacgtagggggcaagcgttatccggatttactgggtgtaaagggagcgtagacagcagcgcaagtctgaagtgaaagcccggggctcaaccccggaatggctttggaaactgtgcagctagagtaccggaggggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 237 - DNA, 253 bp, Artificial Sequence
  Synthetic Polypeptide
tacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 238 - DNA, 253 bp, Artificial Sequence
  Synthetic Polypeptide
tacgtaggtggcgagcgttatccggaatgattgggcgtaaagggtgcgtaggtggcagatcaagtctggagtaaaaggtatgggctcaacccgtacttgctctggaaactgatcagctagagaacagaagaggacggcggaactccatgtgtagcggtaaaatgcgtagatatatggaagaacaccggtggcgaaggcggccgtctggtctggattctgacactgaagcacgaaagcgtggggagcaaatagg

SEQ ID NO: 239 - DNA, 252 bp, Artificial Sequence
  Synthetic Polypeptide
tacgtaggtggcgagcgttatccggaatcattgggcgtaaagagggagcaggcggccgcaaaggtctgtggtgaaagaccgaagctaaacttcggtaagccatggaaaccgggcggctagagtgcggaagaggatcgtggaattccatgtgtagcggtgaaatgcgtagatatatggaggaacaccagtggcgaaggcgacggtctgggccgcaactgacgctcattcccgaaagcgtggggagcaaatagg

SEQ ID NO: 240 - DNA, 252 bp, Artificial Sequence
  Synthetic Polypeptide
tacgtaggtggcgagcgttatccggaatcattgggcgtaaagagggagcaggcggccgcaagggtctgtggtgaaagaccgaagctaaacttcggtaagccatggaaaccgggcggctagagtgcggaagaggatcgtggaattccatgtgtagcggtgaaatgcgtagatatatggaggaacaccagtggcgaaggcgacggtctgggccgcaactgacgctcattcccgaaagcgtggggagcaaatagg

SEQ ID NO: 241 - DNA, 252 bp, Artificial Sequence
  Synthetic Polypeptide
tacgtaggtggcgagcgttatccggaatcattgggcgtaaagagggagcaggcggccgcaagggtctgtggtgaaagaccgaagctaaacttcggtaagccatggaaaccgggcggctagagtgcggaagaggatcgtggaattccatgtgtagcggtgaaatgcgtagatatatggaggaacaccagtggcgaaggcgacggtctgggccgcaactgacgctgattcccgaaagcgtggggagcaaatagg

SEQ ID NO: 242 - DNA, 252 bp, Artificial Sequence
  Synthetic Polypeptide
tacgtaggtggcgagcgttatccggaatcattgggcgtaaagagggagcaggcggccgcaagggtctgtggtgaaagaccgaagctaaacttcggtaagccatggaaaccgggcggctagagtgcggaagaggatcgtggaattccatgtgtagcggtgagatgcgtagatatatggaggaacaccagtggcgaaggcgacggtctgggccgcaactgacgctcattcccgaaagcgtggggagcaaatagg

SEQ ID NO: 243 - DNA, 252 bp, Artificial Sequence
  Synthetic Polypeptide
tacgtagggggcgagcgttatccggaatcattgggcgtaaagagggagcaggcggccgcaagggtctgtggtgaaagaccgaagctaaacttcggtaagccatggaaaccgggcggctagagtgcggaagaggatcgtggaattccatgtgtagcggtgaaatgcgtagatatatggaggaacaccagtggcgaaggcgacggtctgggccgcaactgacgctcattcccgaaagcgtggggagcaaatagg

SEQ ID NO: 244 - DNA, 252 bp, Artificial Sequence
  Synthetic Polypeptide
tacgtaggtggcgagcgttatccggaattattgggcgtaaagagggagcaggcggcactaagggtctgtggtgaaagatcgaagcttaacttcggtaagccatggaaaccgtagagctagagtgtgtgagaggatcgtggaattccatgtgtagcggtgaaatgcgtagatatatggaggaacaccagtggcgaaggcgacgatctggcgcataactgacgctcagtcccgaaagcgtggggagcaaatagg

SEQ ID NO: 245 - DNA, 252 bp, Artificial Sequence
  Synthetic Polypeptide
tacgtaggtggcgagcgttatccggaatcattgggcgtaaagagggagcaggcggccgcaagggtctgtggtgaaagaccgaagctaaacttcggtaagccatggaaaccgggcggctagagtgcggaagaggatcgtggaattccatgtgtagcggtgaaatgcgtagatatatggaggaacaccagtggcgaaggcggcttactggacggtaactgacgttgaggctcgaaagcgtggggagcaaacagg

SEQ ID NO: 246 - DNA, 252 bp, Artificial Sequence
  Synthetic Polypeptide
tacgtaggcggcgagcgttatccggaattattgggcgtaaagagggagcaggcggcactaagggtctgtggtgaaagatcgaagcttaacttcggtaagccatggaaaccgtagagctagagtgtgtgagaggatcgtggaattccatgtgtagcggtgaaatgcgtagatatatggaggaacaccagtggcgaaggcgacgatctggcgcataactgacgctcagtcccgaaagcgtggggagcaaatagg

SEQ ID NO: 247 - DNA, 252 bp, Artificial Sequence
  Synthetic Polypeptide
tacgtaggcggcaagcgttgtccggaattattgggcgtaaagggagcgcaggcgggaaactaagcggatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggctttctggactgtaactgacgctgaggctcgaaagcgagggtagcgaacggg

SEQ ID NO: 248 - DNA, 252 bp, Artificial Sequence
  Synthetic Polypeptide
tacgtaggtggcaagcgttgtccggaattattgggcgtaaagcgcgcgcaggcggataggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacggg

SEQ ID NO: 249 - DNA, 252 bp, Artificial Sequence
  Synthetic Polypeptide
tacgtaggcggcaagcgttgtccggaattattgggcgtaaagggagcgcaggcgggaaactaagcggatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggctttctggactgtaactgacgctgaggctcgaaagtgtgggtagcaaacagg

SEQ ID NO: 250 - DNA, 252 bp, Artificial Sequence
  Synthetic Polypeptide
tacgtaggcggcaagcgttgtccggaattattgggcgtaaagggagcgcaggcgggaaactaagcggatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggcctactgggcaccaactgacgctgaggctcgaaagtgtgggtagcaaacagg

SEQ ID NO: 251 - DNA, 252 bp, Artificial Sequence
  Synthetic Polypeptide
tacgtaggcggcaagcgttgtccggaattattgggcgtaaagggagcgcaggcgggaaactaagcagatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggcttactggacgataactgacgctgaggctcgaaagcgtggggagcaaacagg

SEQ ID NO: 252 - DNA, 252 bp, Artificial Sequence
  Synthetic Polypeptide
tacgtaggtggcaagcgttgtccggaattattgggcgtaaagagcatgtaggcgggcttttaagtctgacgtgaaaatgcggggcttaaccccgtatggcgttggatactggaagtcttgagtgcaggagaggaaaggggaattcccagtgtagcggtgaaatgcgtagatattgggaagaacaccagtggcgaaggcggctttctggactgtaactgacgctgaggctcgaaagctagggtagcgaacggg

SEQ ID NO: 253 - DNA, 253 bp, Artificial Sequence
  Synthetic Polypeptide
tacgtagggggcaagcgttgtccggaataattgggcgtaaagggcgcgtaggcggctcggtaagtctggagtgaaagtcctgcttttaaggtgggaattgctttggatactgtcgggcttgagtgcaggagaggttagtggaattcccagtgtagcggtgaaatgcgtagagattgggaggaacaccagtggcgaaggcgactaactggactgtaactgacgctgaggcgcgaaagtgtggggagcaaacagg

SEQ ID NO: 254 - DNA, 253 bp, Artificial Sequence
  Synthetic Polypeptide
tacgtagggggcgagcgttgtccggaatgattgggcgtaaagggcgcgtaggcggcctgctaagtctgaagtgaaagtcctgctttcaaggtgggaagtgctttggatactggtgggctggagtgcaggagaggaaagcggaattaccggtgtagcggtgaaatgcgtagagatcggtaggaacaccagtggcgaaggcggctttctggactgaaactgacgctgaggcgcgaaagcgtggggagcaaacagg

SEQ ID NO: 255 - DNA, 253 bp, Artificial Sequence
  Synthetic Polypeptide
tacgtagggggcgagcgttgtccggaatgattgggcgtaaagggcgcgtaggcggcctgctaagtctggagtgaaagtcctgctttcaaggtgggaattgctttggatactggtgggctggagtgcaggagaggaaagcggaattaccggtgtagcggtgaaatgcgtagagatcggtaggaacaccagtggcgaaggcggctttctggactgaaactgacgctgaggcgcgaaagcgtggggagcaaacagg

SEQ ID NO: 256 - DNA, 253 bp, Artificial Sequence
  Synthetic Polypeptide
tacgtaggtgacaagcgttgtccggatttactgggtgtaaagggcgcgtaggcggactgtcaagtcagtcgtgaaataccggggcttaaccccggggctgcgattgaaactgacagccttgagtatcggagaggaaagcggaattcctagtgtagcggtgaaatgcgtagatattaggaggaacaccagtggcgaaggcggctttctggacgacaactgacgctgaggcgcgaaagtgtggggagcaaacagg

SEQ ID NO: 257 - DNA, 253 bp, Artificial Sequence
  Synthetic Polypeptide
tacgtagggggcgagcgttgtccggaatgattgggcgtaaagggcgcgtaggcggcctgctaagtctggagtgaaagtcctgctttcaaggtgggaagtgctttggatactggtgggctggagtgcaggagaggaaagcggaattaccggtgtagcggtgaaatgcgtagagatcggtaggaacaccagtggcgaaggcggctttctggactgaaactgacgctgaggcgcgaaagcgtggggagcaaacagg

SEQ ID NO: 258 - DNA, 253 bp, Artificial Sequence
  Synthetic Polypeptide
tacgtagggagcaagcgttatccggatttattgggtgtaaagggtgcgtagacgggacaacaagttagttgtgaaatccctcggcttaactgaggaactgcaactaaaactattgttcttgagtgttggagaggaaagtggaattcctagtgtagcggtgaaatgcgtagatattaggaggaacaccggtggcgaaggcgactttctggacaataactgacgttgaggcacgaaagtgtggggagcaaacagg

SEQ ID NO: 259 - DNA, 253 bp, Artificial Sequence
  Synthetic Polypeptide
tacgtagggagcaagcgttatccggatttattgggtgtaaagggtgcgtagacggtaatgcaagttagttgtgaaatccctcggcttaactgaggaactgcaactaaaactacatttcttgagtattggaggggaaagtggaattcctagtgtagcggtgaaatgcgtagatattaggaggaacaccggtggcgaaggcgactttctggacaattactgacgttgaggcacgaaagtgtggggagcaaacagg

SEQ ID NO: 260 - DNA, 253 bp, Artificial Sequence
  Synthetic Polypeptide
tacgtatggtgcaagcgttatccggatttactgggtgtaaagggagcgtaggtggcaaggcaagccagaagtgaaaacccggggctcaaccgcgggattgcttttggaactgtcatgctagagtgcaggaggggtgagcggaattcctagtgtagcggtgaaatgcgtagatattaggaggaacaccggaggcgaaggcggctcactggactgtaactgacactgaggctcgaaagcgtggggagcaaacagg

SEQ ID NO: 261 - DNA, 252 bp, Artificial Sequence
  Synthetic Polypeptide
tacgtagggggcaagcgttatccggaattattgggcgtaaagagtacgtaggtggttttctaagcacggggtttaaggcaatggcttaaccattgttcgccttgtgaactggaagacttgagtgcaggagaggaaagcggaattcctagtgtagcggtgaaatgcgtagatattaggaggaacaccagtggcgaaggcggctttctggactgtaactgacactgaggtacgaaagcgtggggagcaaacagg

SEQ ID NO: 262 - DNA, 253 bp, Artificial Sequence
  Synthetic Polypeptide
tacgtaggtgacaagcgttgtccggatttactgggtgtaaagggcgcgtaggcggactgtcaagtcagtcgtgaaataccggggcttaaccccggggctgcgattgaaactgacagccttgagtatcggagaggaaagcggaattcctagtgtagcggtgaaatgcgtagatattaggaggaacaccagtggcgaaggcggcttactggacgataactgacgctgaggctcgaaagcgtggggagcaaacagg

SEQ ID NO: 263 - DNA, 253 bp, Artificial Sequence
  Synthetic Polypeptide
tacgtatggtgcaagcgttatccggatttactgggtgtaaagggagcgtaggtggcaaggcaagccagaagtgaaaacccggggctcaaccgcgggattgcttttggaactgtcatgctggagtgcaggaggggtgagcggaattcctagtgtagcggtgaaatgcgtagatattaggaggaacaccggaggcgaaggcggctcactggactgtaactgacactgaggctcgaaagcgtggggagcaaacagg

SEQ ID NO: 264 - DNA, 253 bp, Artificial Sequence
  Synthetic Polypeptide
tacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacgtgtagcagtgaaatgcgtagatatgtggaagaacaccgatggcgaaggcagcctcctgggacataactgacgctcaggcacgaaagcgtggggagcaaacagg

SEQ ID NO: 265 - DNA, 253 bp, Artificial Sequence
  Synthetic Polypeptide
tacgtagggtgcaagcgttaatcggaattactgggcgtaaagcgtgcgcaggcggttctgtaagatagatgtgaaatccccgggctcaacctgggaattgcatatatgactgcaggacttgagtttgtcagaggagggtggaattccacgtgtagcagtgaaatgcgtagatatgtggaagaacaccgatggcgaaggcagccctctgggacatgactgacgctcatgcacgaaagcgtggggagcaaacagg

SEQ ID NO: 266 - DNA, 253 bp, Artificial Sequence
  Synthetic Polypeptide
tacgtagggtgcaagcgttaatcggaattactgggcgtaaagcgtgcgcaggcggttctgtaagatagatgtgaaatccccgggctcaacctgggaattgcatatatgactgcagaacttgagtttgtcagaggagggtggaattccacgtgtagcagtgaaatgcgtagatatgtggaagaacaccgatggcgaaggcagccctctgggacatgactgacgctcatgcacgaaagcgtggggagcaaacagg

SEQ ID NO: 267 - DNA, 253 bp, Artificial Sequence
  Synthetic Polypeptide
tacggagggtgcaagcgttaatcggaatcactgggcgtaaagcgcacgtaggctgttatgtaagtcaggggtgaaatcccacggctcaaccgtggaactgcccttgatactgcacgacttgaatccgggagagggtggcggaattccaggtgtaggagtgaaatccgtagatatctggaggaacatcagtggcgaaggcggccacctggaccggtattgacgctgaggtgcgaaagcgtggggagcaaacagg

SEQ ID NO: 268 - DNA, 252 bp, Artificial Sequence
  Synthetic Polypeptide
tacgtatggagcaagcgttatccggatttattgggcgtaaagcgcgcgtaggcggcttgttaagcaaggtcttaaatcttggggctcaacctcaagccggatcttgaactggcaggctcgagtatggtagaggaaagtggaattcccagtgtagcggtggaatgcgcagatattgggaagaacaccgatggcgaaggcagctttctgggccattactgacgctgaggtgcgaaagctaggggagcaaacagg

(19) United States
(12) Patent Application Publication
(10) Pub. No.: US 2020/0360450 A1
(43) Pub. Date: Nov. 19, 2020

(54) TREATMENT OF LIVER DISEASE BY MODULATION OF THE MICROBIOME

(71) Applicant: Finch Therapeutics, Inc., Somerville, MA (US)

(72) Inventors: Sonia TIMBERLAKE, Somerville, MA (US); Ylaine GERARDIN, Somerville, MA (US)

(21) Appl. No.: 16/636,837

(22) PCT Filed: Aug. 7, 2018
(86) PCT No.: PCT/US18/45595

§ 371 Date: Feb. 5, 2020

Related U.S. Application Data
(60) Provisional application No. 62/644,236, filed on Mar. 16, 2018.
(60) Provisional application No. 62/542,038, filed on Aug. 7, 2017.

(51) Int. Cl. A61K 35/747; A61K 35/745; A61K 35/741; A61K 35/742; A61P 1/16

(52) U.S. Cl. CPC A61K 35/747; A61K 35/745; A61P 1/16; A61K 35/742; A61K 35/741

(57) ABSTRACT

The present invention relates to, in part, compositions and methods for delivery of mixtures of bacterial strains for treating and/or preventing Primary Sclerosing Cholangitis (PSC).

### PRIORITY

This application claims the benefit of and priority to U.S. 62/542,038, filed Aug. 7, 2017 and U.S. 62/644,236, filed Mar. 16, 2018. The contents of the aforementioned applications are hereby incorporated by reference in their entirety.

### FIELD OF THE INVENTION

The present invention relates to, in part, compositions and methods useful for halting progression of and/or treating Primary Sclerosing Cholangitis (PSC).

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: FIN-006PC-Sequence_Listing_ST25; date recorded: Aug. 7, 2018; file size: 136,513 bytes).

### BACKGROUND

Primary sclerosing cholangitis (PSC) is a rare but serious chronic disease that affects approximately 25,000 patients in the United States. The cause of PSC is currently unknown. PSC is characterized by inflammation of the bile ducts that leads to biliary scarring and blockage, impairing liver function and causing liver damage. While many patients with PSC will initially be asymptomatic, as the disease progresses, patients may experience a range of symptoms including pruritus, jaundice, fatigue, dark urine, fat malabsorption, and other symptoms of liver dysfunction. Patients will ultimately progress to terminal liver failure, requiring a liver transplant. There is no currently-available therapy for treating PSC, other than liver transplant. Accordingly, there remains an unmet need for a therapy that treats and/or prevents PSC.

### SUMMARY OF THE INVENTION

The present invention is based, in part, on the discovery that pharmaceutical compositions comprising fresh, frozen, dried, or reconstituted feces from at least one healthy human donor who satisfies at least one selection criterion, as described herein, and/or comprising novel mixtures of bacterial strains diminish inflammation in the bile ducts, halt the progression of PSC, and/or treat PSC.

In various aspects, the present invention relates to a pharmaceutical composition comprising a bacterial mixture wherein at least one bacterial strain in the bacterial mixture comprises a 16S V4 sequence that is greater than about 97% identical to the 16S V4 sequence of any one of the operational taxonomic units (OTUs) recited in Table 1.

In various embodiments, the 16S V4 sequence of the at least one bacterial strain in the bacterial mixture is greater than about 98%, 99%, or 99.5% identical to the 16S V4 sequence of any one of the OTUs recited in Table 1. In various embodiments, the 16S V4 sequence of the at least one bacterial strain in the bacterial mixture is identical to the 16S V4 sequence of any one of the OTUs recited in Table 1.

In various embodiments, the bacterial mixture comprises a fecal microbiota preparation that comprises a donor's entire or substantially complete fecal microbiota. In one aspect, a fecal microbiota preparation comprises a non-selected fecal microbiota. In another aspect, a fecal microbiota preparation comprises an isolated or purified population of live non-pathogenic fecal bacteria. In a further aspect, a fecal microbiota preparation comprises a non-selected and substantially complete fecal microbiota preparation from a single donor. In such embodiments, at least one bacterial strain in the bacterial mixture comprises a 16S V4 sequence that is greater than about 97% identical to the 16S V4 sequence of any one of the operational taxonomic units (OTUs) recited in Table 1.

In various embodiments, the at least one bacterial strain is a commensal bacterial strain.

In various embodiments, the at least one bacterial strain is obtained from one or more human beings, e.g., healthy human beings and/or who satisfy at least one selection criterion.

In various embodiments, the at least one selection criterion comprises a donor having fecal material which lacks or has a low abundance of bacteria that are specifically found in fecal material originating from a PSC patient.

In various embodiments, the at least one selection criterion comprises the number of priority bacterial strains and/or their relative abundance in a donor's stool, wherein the priority bacterial strains are identified in Table 1 as having a 16S V4 sequence of one of SEQ ID NO: 1 to SEQ ID NO: 32.

In various embodiments, the at least one selection criterion comprises the number of priority clusters and/or their relative abundance in a donor's stool, wherein the priority clusters are identified in Table 1 as having a 16S V4 sequence that is at least 97% identical to one of SEQ ID NO: 10, SEQ ID NO: 13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240.

In various embodiments, the donor's stool comprises a least about five (e.g., about seven and about twelve) of the priority clusters identified in Table 1.

In various embodiments, the donor's stool comprises at least one (e.g., at least about five, at least about ten, at least about fifteen, at least about twenty, at least about twenty-five, at least about thirty, and about thirty-five) bacterial strains which comprise a 16S V4 sequence that is greater than about 97% identical (e.g., about identical) to one of SEQ ID NO: 1 to SEQ ID NO: 32, SEQ ID NO: 60, SEQ ID NO: 238, or SEQ ID NO: 240.

In various embodiments, the donor's stool comprises a relative abundance of priority bacterial strains and/or priority clusters greater than about 0.01% (e.g., greater than about 0.05% and greater than about 0.1%) of the total stool bacterial community.

In various embodiments, a donor is selected for being in the top quartile (e.g., top 10th percentile) based on the number of priority bacterial strains and/or priority clusters and abundance thereof in their stool relative to other healthy, screened, pathogen-free potential donors.

In various embodiments, the at least one selection criterion comprises the presence of one or more (e.g., two, three, four, five, six, seven, eight, nine, ten, eleven, and twelve) of the following bacterial strains in a donor's stool: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

In various embodiments, the at least one selection criterion comprises the absence of Primary Sclerosing Cholangitis (PSC) or the absence of symptoms of PSC.

In various embodiments, the at least one bacterial strain is obtained from one human being or from more than one human being.

In various embodiments, the source material is fresh, frozen, dried, or reconstituted feces. In various embodiments, the at least one bacterial strain is obtained from a laboratory stock or bacterial cell bank.

In various embodiments, the at least one bacterial strain is isolated and/or purified from its source material or is not isolated and/or purified from its source material prior to forming the bacterial mixture.

In various embodiments, the at least one bacterial strain is cultured prior to forming the bacterial mixture or is not cultured prior to forming the bacterial mixture.

In various embodiments, the at least one bacterial strain is isolated and/or purified from its source material prior to forming the bacterial mixture.

In various embodiments, the bacterial mixture comprises at least two (e.g., five, ten, twenty, thirty, forty, and fifty) bacterial strains comprising a 16S V4 sequence that is greater than about 97% identical to the 16S V4 sequence of one of the OTUs recited in Table 1.

In various embodiments, the bacterial mixture comprises at least two (e.g., five, ten, twenty, thirty, forty, and fifty) bacterial strains, wherein each bacterial strain in the bacterial mixture comprises a 16S V4 sequence that is greater than about 97% identical to the 16S V4 sequence of one of the OTUs recited in Table 1.

In various embodiments, the bacterial mixture comprises between about five and about one hundred (e.g., between about ten and about seventy-five, between about fifteen and about fifty, between about twenty and about forty-five, between about twenty-five and about forty, and between about thirty and about thirty-five) bacterial strains in the bacterial mixture, wherein a plurality of the bacterial strains comprise a 16S V4 sequence that is greater than about 97% identical to the 16S V4 sequence of one of the OTUs recited in Table 1.

In various embodiments, at least one bacterial strain (e.g., a plurality of bacterial strains) is included in the bacterial mixture due its greater abundance in the GI tract of a healthy subject relative to its abundance in the GI track of a subject with PSC and/or due to its greater abundance in feces from a healthy subject relative to its abundance in feces from a subject with PSC.

In various embodiments, at least one bacterial strain (e.g., a plurality of bacterial strains) is included in the bacterial mixture due to its ability to engraft in the GI tract of a PSC patient.

In various embodiments, at least one bacterial strain (e.g., a plurality of bacterial strains) is included in the bacterial mixture due to its ability to improve levels in the liver biomarker Alkaline Phosphatase (ALP).

In various embodiments, at least one bacterial strain (e.g., a plurality of bacterial strains) is included in the bacterial mixture due to its ability to reduce inflammation in the bile duct and/or in the liver.

In various embodiments, at least one (e.g., at least about five, at least about ten, at least about fifteen, at least about twenty, at least about twenty-five, at least about thirty, and about thirty-five) bacterial strain included in the bacterial mixture comprises a 16S V4 sequence that is greater than about 97% identical (e.g., about identical) to one of SEQ ID NO: 1 to SEQ ID NO: 32, SEQ ID NO: 60, SEQ ID NO: 238, or SEQ ID NO: 240.

In various embodiments, the mixture of bacterial strains comprises one or more (e.g., two, three, four, five, six, seven, eight, nine, ten, eleven, and twelve) of the following bacterial strains: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

In various embodiments, the pharmaceutical composition further comprises a pharmaceutically acceptable excipient.

In various embodiments, the pharmaceutical composition is formulated for oral administration and/or for delivery of the bacterial mixture to an intestine, e.g., the small intestine and/or the large intestine, e.g., including the cecum. In various embodiments, delivery of the pharmaceutical composition is substantially completed prior to the rectum.

In various embodiments, the pharmaceutical composition is formulated as a capsule, e.g., which comprises a delayed-release coating.

In various embodiments, a plurality of the bacterial strains in the bacterial mixture is live, vegetative cells and/or lyophilized cells.

In various embodiments, a plurality of the bacterial strains in the bacterial mixture is spores.

In various embodiments, a plurality of the bacterial strains in the bacterial mixture is non-pathogenic bacteria. In various embodiments, each bacterial strain in the bacterial mixture is a non-pathogenic bacterium.

In various embodiments, the pharmaceutical composition is capable of treating or preventing PSC in a subject, e.g., a human subject.

In various aspects, the present invention relates to a method for treating or preventing PSC. The method comprises a step of administering to a subject in need thereof an effective amount of a pharmaceutical composition of any aspect or embodiment disclosed herein. In embodiments, following administration of the pharmaceutical composition, the subject's microbiome diversity changes towards the diversity present in the pharmaceutical composition. In various embodiments, the administering an effective amount of the pharmaceutical composition reduces inflammation of the bile duct and/or the liver.

In various aspects, the present invention relates to a method for treating or preventing Primary Sclerosing Cholangitis (PSC) in a patient in need thereof. The method comprises a step of administering an effective amount of fresh, frozen, dried, or reconstituted feces from at least one healthy human donor, wherein the at least one healthy human donor satisfies at least one selection criterion.

In various embodiments, the at least one selection criterion comprises a donor having fecal material which lacks or has a low abundance of bacteria that are specifically found in fecal material originating from a PSC patient.

In various embodiments, the at least one selection criterion comprises the number of priority bacterial strains and/or their relative abundance in a donor's stool, wherein the priority bacterial strains are identified in Table 1 as having a 16S V4 sequence of one of SEQ ID NO: 1 to SEQ ID NO: 32.

In various embodiments, the at least one selection criterion comprises the number of priority clusters and/or their relative abundance in a donor's stool, wherein the priority clusters are identified in Table 1 as having a 16S V4 sequence that is at least 97% identical to one of SEQ ID NO: 10, SEQ ID NO: 13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240. In various embodiments, the presence of a priority cluster and its relative abundance in each donor can be determined by counting the number of sequencing reads with more than 97% identity to the priority clusters' sequences identified in Table 1.

In various embodiments, the donor's stool comprises a least about five (e.g., seven and twelve) of the priority clusters identified in Table 1.

In various embodiments, the donor's stool comprises at least one (e.g., at least about five, at least about ten, at least about fifteen, at least about twenty, at least about twenty-five, at least about thirty, and about thirty-five) bacterial strain which comprise a 16S V4 sequence that is greater than about 97% identical (e.g., about identical) to one of SEQ ID NO: 1 to SEQ ID NO: 32, SEQ ID NO: 60, SEQ ID NO: 238, or SEQ ID NO: 240.

In various embodiments, the donor's stool comprises a relative abundance of priority bacterial strains and/or the priority cluster greater than about 0.01% (e.g., 0.05% and 0.1%) of the total stool bacterial community.

In various embodiments, a donor is selected for being in the top quartile (e.g., top 10th percentile) based on the number of priority bacterial strains and/or the priority clusters and abundance thereof in their stool relative to other healthy, screened, pathogen-free potential donors.

In various embodiments, the at least one selection criterion comprises the presence of one or more (e.g., two, three, four, five, six, seven, eight, nine, ten, eleven, and twelve) of the following bacterial strains in a donor's stool: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

In various embodiments, the at least one selection criterion comprises the absence of Primary Sclerosing Cholangitis (PSC) or the absence of symptoms of PSC.

In various embodiments, the effective amount of fresh, frozen, dried, or reconstituted feces reduces inflammation of the bile duct and/or the liver.

In various embodiments, the effective amount of fresh, frozen, dried, or reconstituted feces improves levels of the liver biomarker Alkaline Phosphatase (ALP).

In various embodiments, the fresh, frozen, dried, or reconstituted feces is obtained from one healthy human donor.

In various embodiments, the fresh, frozen, dried, or reconstituted feces is obtained from more than one healthy human donor.

In various embodiments, the fresh, frozen, dried, or reconstituted feces comprises spores and/or live, vegetative cells.

In various embodiments, the fresh, frozen, dried, or reconstituted feces comprises a plurality of non-pathogenic bacteria.

In various embodiments, the fresh, frozen, dried, or reconstituted feces comprises a plurality of bacterial strains having greater abundances relative to their abundances in fresh, frozen, dried, or reconstituted feces from a subject with PSC.

In various embodiments, the fresh, frozen, dried, or reconstituted feces comprises at least one bacterial strain capable of engrafting in the GI tract of a PSC patient.

In various embodiments, the method further comprises administering at least one isolated, purified, and/or cultured bacterial strain comprising a 16S V4 sequence that is greater than about 97% identical to the 16S V4 sequence of any one of the operational taxonomic units (OTUs) recited in Table 1.

In various embodiments, the method further comprises administering a pharmaceutically acceptable excipient combined with the fresh, frozen, dried, or reconstituted feces.

In various embodiments, following administration of the donor's stool, the PSC patient's microbiome diversity changes towards the diversity present in the donor's stool.

In various embodiments, the patient in need thereof is a human.

In various aspects, the present invention relates to a method for manufacturing a pharmaceutical composition of any aspect or embodiment disclosed herein. The method comprises a step of obtaining at least one bacterial strain comprising a 16S V4 sequence that is greater than about 97% identical to the 16S V4 sequence of any one of the operational taxonomic units (OTUs) recited in Table 1 and formulating the least one bacterial strain into a pharmaceutical composition.

In various embodiments, the at least one bacterial strain is contained in fresh, frozen, dried, or reconstituted feces obtained from one or more healthy human beings who satisfy at least one selection criterion.

In various embodiments, the at least one selection criterion comprises a donor having fecal material which lacks or has a low abundance of bacteria that are specifically found in fecal material originating from a PSC patient.

In various embodiments, the at least one selection criterion comprises the number of priority bacterial strains and/or their relative abundance in a donor's stool, wherein the priority bacterial strains are identified in Table 1 as having a 16S V4 sequence of one of SEQ ID NO: 1 to SEQ ID NO: 32.

In various embodiments, the at least one selection criterion comprises the number of priority clusters and/or their relative abundance in a donor's stool, wherein the priority clusters are identified in Table 1 as having a 16S V4 sequence that is at least 97% identical to one of SEQ ID NO: 10, SEQ ID NO: 13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240. In various embodiments, the presence of a priority cluster and its relative abundance in each donor can be determined by counting the number of sequencing reads with more than 97% identity to the priority clusters' sequences identified in Table 1.

In various embodiments, the donor's stool comprises a least about five (e.g., seven and twelve) of the priority clusters identified in Table 1.

In various embodiments, the donor's stool comprises at least one (e.g., at least about five, at least about ten, at least about fifteen, at least about twenty, at least about twenty-five, at least about thirty, and about thirty-five) bacterial strain which comprise a 16S V4 sequence that is greater than about 97% identical (e.g., about identical) to one of SEQ ID NO: 1 to SEQ ID NO: 32, SEQ ID NO: 60, SEQ ID NO: 238, or SEQ ID NO: 240.

In various embodiments, the donor's stool comprises a relative abundance of priority bacterial strains and/or the priority cluster greater than about 0.01% (e.g., 0.05% and 0.1%) of the total stool bacterial community.

In various embodiments, a donor is selected for being in the top quartile (e.g., top 10th percentile) based on the number of priority bacterial strains and/or the priority clusters and abundance thereof in their stool relative to other healthy, screened, pathogen-free potential donors.

In various embodiments, the at least one selection criterion comprises the presence of one or more (e.g., two, three, four, five, six, seven, eight, nine, ten, eleven, and twelve) of the following bacterial strains in a donor's stool: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

In various embodiments, the at least one selection criterion comprises the absence of Primary Sclerosing Cholangitis (PSC) or the absence of symptoms of PSC.

In various aspects, the present invention relates to method for manufacturing a pharmaceutical composition suitable for the treatment of PSC The method comprises steps of screening a potential human feces donor for the presence of at least one selection criterion; selecting a potential human feces donor as a human feces donor based upon the presence of the at least one selection criterion; obtaining feces from the human feces donor; and formulating the obtained feces into a pharmaceutical composition for administration to a PSC patient.

In various embodiments, the at least one selection criterion comprises the number of priority bacterial strains and/or their relative abundance in a donor's stool, wherein the priority bacterial strains are identified in Table 1 as having a 16S V4 sequence of one of SEQ ID NO: 1 to SEQ ID NO: 32.

In various embodiments, the at least one selection criterion comprises the number of priority clusters and/or their relative abundance in a donor's stool, wherein the priority clusters are identified in Table 1 as having a 16S V4 sequence that is at least 97% identical to one of SEQ ID NO: 10, SEQ ID NO: 13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240. In various embodiments, the presence of a priority cluster and its relative abundance in each donor can be determined by counting the number of sequencing reads with more than 97% identity to the priority clusters' sequences identified in Table 1.

In various embodiments, the donor's stool comprises a least about five (e.g., seven and twelve) of the priority clusters identified in Table 1.

In various embodiments, the donor's stool comprises at least one (e.g., at least about five, at least about ten, at least about fifteen, at least about twenty, at least about twenty-five, at least about thirty, and about thirty-five) bacterial strain which comprise a 16S V4 sequence that is greater than about 97% identical (e.g., about identical) to one of SEQ ID NO: 1 to SEQ ID NO: 32, SEQ ID NO: 60, SEQ ID NO: 238, or SEQ ID NO: 240.

In various embodiments, the donor's stool comprises a relative abundance of priority bacterial strains and/or the priority cluster greater than about 0.01% (e.g., 0.05% and 0.1%) of the total stool bacterial community.

In various embodiments, a donor is selected for being in the top quartile (e.g., top 10th percentile) based on the number of priority bacterial strains and/or the priority clusters and abundance thereof in their stool relative to other healthy, screened, pathogen-free potential donors.

In various embodiments, the at least one selection criterion comprises the presence of one or more (e.g., two, three, four, five, six, seven, eight, nine, ten, eleven, and twelve) of the following bacterial strains in a donor's stool: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

In various embodiments, the at least one selection criterion comprises the absence of Primary Sclerosing Cholangitis (PSC) or the absence of symptoms of PSC. In various embodiments, the at least one selection criterion comprises a donor having fecal material which lacks or has a low abundance of bacteria that are specifically found in fecal material originating from a PSC patient.

In various embodiments, the obtained feces is fresh, frozen, dried, or reconstituted feces.

In various embodiments, the pharmaceutical composition further comprises at least one bacterial strain that is isolated, purified, and/or cultured.

In various embodiments, the at least bacterial strain comprises a 16S V4 sequence that is greater than about 97% identical to the 16S V4 sequence of any one of the operational taxonomic units (OTUs) recited in Table 1.

In various embodiments, the pharmaceutical composition further comprises a pharmaceutically acceptable excipient.

In various aspects, the present invention relates to a method for manufacturing a pharmaceutical composition suitable for the treatment of PSC. The method comprises steps of screening a plurality of potential human feces donors for the presence of at least one selection criterion; selecting a plurality of potential human feces donor as human feces donors based upon the presence of the at least one selection criterion; obtaining feces from the human feces donors; and formulating the obtained feces into a pharmaceutical composition for administration to a PSC patient.

In various embodiments, the at least one selection criterion comprises a donor having fecal material which lacks or has a low abundance of bacteria that are specifically found in fecal material originating from a PSC patient.

In various embodiments, the at least one selection criterion comprises the number of priority bacterial strains and/or their relative abundance in a donor's stool, wherein the priority bacterial strains are identified in Table 1 as having a 16S V4 sequence of one of SEQ ID NO: 1 to SEQ ID NO: 32.

In various embodiments, the at least one selection criterion comprises the number of priority clusters and/or their relative abundance in a donor's stool, wherein the priority clusters are identified in Table 1 as having a 16S V4 sequence that is at least 97% identical to one of SEQ ID NO: 10, SEQ ID NO: 13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240. In various embodiments, the presence of a priority cluster and its relative abundance in each donor can be determined by counting the number of sequencing reads with more than 97% identity to the priority clusters' sequences identified in Table 1.

In various embodiments, the donor's stool comprises a least about five (e.g., seven and twelve) of the priority bacterial strains and/or the priority clusters identified in Table 1.

In various embodiments, the donor's stool comprises a relative abundance of priority bacterial strains and/or the priority cluster greater than about 0.01% (e.g., 0.05% and 0.1%) of the total stool bacterial community.

In various embodiments, a donor is selected for being in the top quartile (e.g., top 10th percentile) based on the number of priority bacterial strains and/or the priority clusters and abundance thereof in their stool relative to other healthy, screened, pathogen-free potential donors.

In various embodiments, the at least one selection criterion comprises the presence of one or more (e.g., two, three, four, five, six, seven, eight, nine, ten, eleven, and twelve) of the following bacterial strains in a donor's stool: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

In various embodiments, the at least one selection criterion comprises the absence of Primary Sclerosing Cholangitis (PSC) or the absence of symptoms of PSC.

In various embodiments, the obtained feces is fresh, frozen, dried, or reconstituted feces.

In various embodiments, the pharmaceutical composition further comprises at least one bacterial strain that is isolated, purified, and/or cultured.

In various embodiments, the at least bacterial strain comprises a 16S V4 sequence that is greater than about 97% identical to the 16S V4 sequence of any one of the operational taxonomic units (OTUs) recited in Table 1.

In various embodiments, the pharmaceutical composition further comprises a pharmaceutically acceptable excipient.

Any aspect or embodiment described herein can be combined with any other aspect or embodiment as disclosed herein.

### BRIEF DESCRIPTION OF THE DRAWINGS

FIG. 1A is a graph showing specific changes in patients' microbiome pre-FMT versus 1 week post-FMT.

FIG. 1B is a metric multidimensional scaling (MDS) plot of Jensen-Shannon divergence. Changes in diversity for patient's pre-FMT and post-FMT is shown.

FIG. 1C is a graph showing changes in the diversity of patient's microbiome (as measured by the Shannon Diversity Index) at 1 week following FMT.

FIG. 1D is a graph showing changes in patient's microbiome relative to the diversity of the donor microbiome (as measured by 1.0 minus the Jensen-Shannon divergence value) at 1 week following FMT.

FIG. 1E is a graph showing changes over time in the diversity of patient's microbiome up to 24 weeks following FMT.

FIG. 1F is a graph showing changes over time in patient's microbiome relative to the diversity of the donor microbiome up to 24 weeks following FMT.

FIG. 1G is a graph showing changes in diversity across the ten patients (as measured by the Shannon Diversity Index).

FIG. 2A is a graph showing changes over time in the total abundance of engrafters as a fraction of community

FIG. 2B and FIG. 2C are graphs showing specific engrafting bacterial strains (at the Family level). FIG. 2C shows data for the top represented bacterial families.

FIG. 2D is a pie chart showing distribution of engrafting bacterial strains (at the Class level).

FIG. 2E is a graph showing Spearman's rank correlating engrafting strains and liver function.

FIG. 2F is a graph showing Spearman rank correlating the twenty-four top frequent engrafting strains and liver function.

FIG. 3 is a graph showing literature-identified taxa and changes in abundance between patents with PSC and healthy subjects. In particular, the literature shows that Rothia, Veillonella, and Fusobacterium are enriched in the microbiome of PSC patients whereas PSC patients have a deficit in Coprococcus, Clostridiales, Phascolarctobacterium, and Christensenellaceae.

### DETAILED DESCRIPTION

The present invention is based, in part, on the discovery that inflammation in the bile ducts can be diminished, progression of Primary Sclerosing Cholangitis (PSC) can be halted, and PSC can be treated, thereby avoiding or delaying terminal liver failure that results from PSC, by administering pharmaceutical compositions comprising fresh, frozen, dried, or reconstituted feces from at least one healthy human donor who satisfies at least one selection criterion, as described herein, and/or comprising novel mixtures of bacterial strains.

While the causes of PSC are not all fully understood, a dysbiotic microbiome is strongly implicated in the disease's etiology. There are strong links between inflammatory bowel disease (IBD) and PSC, with 70% of PSC patients having some form of IBD, albeit usually mild. Microbiome landscape studies have shown that the microbiome of PSC patients is distinct from both healthy patients and non-PSC IBD patients (which includes over 98% of all patients with IBD). Relative to healthy individuals, the PSC microbiome has low diversity—an indicator usually associated with disease. Finally, antibiotic therapies have been observed to have a significant impact on key liver function blood markers associated with PSC, indicating that a disruption of the microbiome through antibiotics may temporarily disrupt disease activity.

In some embodiments, restoring a healthy gut community is an effective therapy for halting the inflammatory tissue injury, scarring, and clinical symptoms associated with PSC.

In various embodiments, fresh, frozen, dried, or reconstituted feces from at least one healthy human donor who satisfies at least one selection criterion, as described herein, are formulated into a pharmaceutical composition for delivery to the colon for the treatment of PSC. For example, a composition described herein can comprise a non-selected or substantially complete fecal microbiota preparation from one or more human donors. Herein a non-selected fecal microbiota refers to a community or mixture of fecal microbes derived from a donor's fecal sample without selection and substantially resembling microbial constituents and population structure found in such fecal sample. As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5%.

In various embodiments, novel mixtures of bacterial strains are formulated into a pharmaceutical composition for delivery to the colon for the treatment of PSC.

In various embodiments, fresh, frozen, dried, or reconstituted feces from at least one healthy human donor who satisfies at least one selection criterion, as described herein, and novel mixtures of bacterial strains are formulated into a pharmaceutical composition for delivery to the colon for the treatment of PSC.

The pharmaceutical compositions of the present invention halt progression of PSC and/or symptoms of PSC and improve a patient's quality of life by improving liver function serum biomarkers, which are surrogate endpoints that are associated with long-term disease outcomes (liver failure requiring liver transplant or fatal liver disease). Without wishing to be bound by theory, the present invention replaces the dysbiotic gut microbiome with a healthy community (and which has greater diversity than the dysbiotic gut, e.g., the gut of a subject with PSC), reducing bile duct inflammation, and improving liver function in a patient with PSC, thereby preventing and/or treating PSC.

In some embodiments, a pharmaceutical composition is a minimally processed fecal product from donors, selected for treatment of PSC based on the presence and abundance of certain bacterial strains in their colon. In some embodiments, a pharmaceutical composition is a minimally processed fecal product from donors, selected for treatment of PSC based on their satisfaction of selection criteria, as described herein. For example, a pharmaceutical composition can comprise a substantially complete or non-selected fecal microbiota from a single donor, selected for treatment of PSC based on the presence and abundance of certain bacterial strains in their colon, or the absence of certain bacterial strains in their colon.

In embodiments, a pharmaceutical composition further includes or comprises isolated, purified, and/or cultured bacterial strains. Without wishing to be bound by theory, the pharmaceutical compositions reduce symptoms and disease severity observed in a fecal microbiota transplant (FMT) interventional studies. Broadly, these pharmaceutical compositions are defined, in part, by: 1) fecal material originating from a healthy donor, 2) bacterial strains ability to engraft in a PSC patient, and/or 3) bacterial strains associated with improvement in alkaline phosphatase (ALP), which is the most clinically-relevant liver function biomarker. There is significant heterogeneity among individuals' gut microbiota; thus, a method for selecting donors specifically for treating PSC increases the probability that these mixture of bacterial strains will engraft in the patient gut and provide a clinical response.

Examples of microbial strains, present in the feces of a selected donor and/or from an isolated, purified, and/or cultured source, and which are useful in the present invention, are listed in Table 1.

Donor Selection Criteria

A donor who satisfies at least one selection criterion, as described herein, will have feces comprising at least one of the bacterial strains or a plurality of bacterial strains described in the next section.

In various embodiments, a selection criterion is fecal material (originating from a donor) which lacks or has a low abundance of bacteria that are specifically found in fecal material originating from a PSC patient, e.g., bacteria known to be in or found in fecal material from a PSC patient and not in fecal material from a healthy person (who does not have PSC).

In various embodiments, a selection criterion relates to the number of priority bacterial strains and/or the priority clusters and their relative abundance in a donor's stool microbiome.

In various embodiments, the number of priority bacterial strains and/or the priority clusters and their relative abundance in a donor's stool may be characterized by the presence of genetic markers (e.g., specific nucleotide sequences) associated with specific bacterial strains. Identification of these genetic markers can be performed by high throughput DNA sequencing.

In various embodiments, the priority bacterial strains in Table 1 are identifiable as having a 16S V4 sequence of one of SEQ ID NO: 1 to SEQ ID NO: 32. The priority strains are identified by their stronger associations with decreases in the three main liver function tests (e.g., ALP, ALT, and AST).

In various embodiments, the priority clusters in Table 1 are identifiable as having a 16S V4 sequence that is at least 97% identical to one of SEQ ID NO: 10, SEQ ID NO: 13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240. In various embodiments, the presence of a priority cluster and its relative abundance in each donor can be determined by counting the number of sequencing reads with more than 97% identity to the clusters' sequences identified in Table 1.

In various embodiments, the donor's stool comprises a least about five (e.g., seven and twelve) of the priority clusters identified in Table 1.

In various embodiments, the donor's stool comprises at least one (e.g., at least about five, at least about ten, at least about fifteen, at least about twenty, at least about twenty-five, at least about thirty, and about thirty-five) bacterial strain which comprise a 16S V4 sequence that is greater than about 97% identical (e.g., about identical) to one of SEQ ID NO: 1 to SEQ ID NO: 32, SEQ ID NO: 60, SEQ ID NO: 238, or SEQ ID NO: 240.

In various embodiments, the donor's stool comprises a relative abundance of priority bacterial strains and/or priority clusters greater than about 0.01% (e.g., greater than about 0.05% and greater than about 0.1%) of the total stool bacterial community.

In various embodiments, a donor is selected for being in the top quartile (e.g., top 10th percentile) based on the number of priority bacterial strains and/or priority clusters and abundance thereof in their stool relative to other healthy, screened, pathogen-free potential donors.

In embodiments, bacterial strains in the priority clusters include Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

In an embodiment, a bacterial strain is included in a bacterial mixture based, in part, on its abundance in donors whose feces was used for successful or unsuccessful fecal microbiota transplants (FMTs) for treating PSC.

In an embodiment, a bacterial strain is included in a bacterial mixture based, in part, on its presence in the fecal samples of donors whose feces was used for FMTs for treating PSC and which provided a therapeutically effective result in the PSC patient.

In embodiments, a bacterial strain is included in a bacterial mixture based, in part, on its depletion in subjects with PSC. For example, it has been reported the following bacterial strains are depleted in subjects with PSC: Bacteria, Firmicutes, Clostridia, Clostridiales, Lachnospiraceae, Blautia; Bacteria, Firmicutes, Clostridia, Clostridiales, Lachnospiraceae, Coprococcus; Bacteria, Firmicutes, Clostridia, Clostridiales, Lachnospiraceae, Roseburia; Bacteria, Firmicutes, Negativicutes, Selenomonadales, Veillonellaceae, Veillonella; and Bacteria, Proteobacteria, Deltaproteobacteria, Desulfovibrionales, Desulfovibrionaceae, Desulfovibrio. See, e.g., FIG. 3, which shows literature-identified taxa and changes in abundance between patents with PSC and healthy subjects.

In an embodiment, a bacterial strain is included in a bacterial mixture based, in part, on its ability to engraft in a recipient. For example, the recipient may be a FMT recipient who received fecal transplant from a donor. The bacterial strain is considered to successfully engraft if the strain is abundant in donors and also increased in recipient patients.

In various embodiments, a bacterial strain is included in a bacterial mixture based, in part, on its presence in a healthy, non-PSC individual. Alternately, a bacterial strain may be selected for inclusion in the bacterial mixture based, in part, on its absence or reduced levels in a subject with PSC.

In various embodiments, a bacterial strain is included in a bacterial mixture based, in part, on its association with improvement in Alkaline Phosphatase (ALP).

In various embodiments, a bacterial strain is included in a bacterial mixture based, in part, on its ability to provide systemic and/or localized anti-inflammatory and/or immunoregulatory effects. An example of a localized anti-inflammatory effect is reducing inflammation of the bile ducts and/or of the liver.

In various embodiments, a bacterial strain is included in a bacterial mixture based, in part, on its ability to directly inhibit a pathogenic bacterium through production of a secreted product.

In some embodiments, a bacterial strain is included in a bacterial mixture based, in part, on its ability to directly compete with the pathogenic bacteria for a niche

In some embodiments, a bacterial strain is included in a bacterial mixture based, in part, on its ability to help maintain and/or repair a deficient gut barrier.

In embodiments, a bacterial strain is included in a bacterial mixture based, in part, on its ability to activate Toll-Like Receptors (TLRs), which modulate the production of antimicrobial peptides, which target many human bacterial pathogens.

In embodiments, a bacterial strain is included in a bacterial mixture based, in part, on its ability to induce a thickening of the colonic epithelial mucus.

In embodiments, a bacterial strain is included in a bacterial mixture based, in part, on its ability to decolonize a pathogenic bacterium.

In some embodiments, a bacterial strain is included in a bacterial mixture based, in part, on its ability to induce an increase in IgA production.

In embodiments, a bacterial strain is included in a bacterial mixture based, in part, on its ability to eradicate a pathogenic bacterium.

In embodiments, a bacterial strain is included in a bacterial mixture based, in part, on its ability to improve tight junction integrity.

In some embodiments, a bacterial is included in a bacterial mixture based, in part, on its ability to induce an increase in antimicrobial peptide production.

In embodiments, a bacterial strain is included in a bacterial mixture based, in part, on its ability to induce improved tight junction integrity.

In embodiments, a bacterial strain is included in a bacterial mixture based, in part, on its ability to produce Short-Chain Fatty Acid (SCFAs) or its ability to enhance production of SCFAs, which increase the thickness of the mucus layer, maintain the health of colonocytes, and induce IgA production. As used herein, SCFAs refer to fatty acids with an aliphatic tail of less than six carbon atoms. Illustrative SCFAs include, but are not limited to, formic acid, acetic acid, propionic acid, butyric acid, isobutyric acid, valeric acid, and isovaleric acid.

In an embodiment, a bacterial strain is included in a bacterial mixture based, in part, on its ability to promote restoration of mucosal barrier functions.

In some embodiments, the pharmaceutical composition of the invention includes a bacterial strain that prevents and/or reduces the loss of mucus thickness associated with various GI disorders. In some embodiments, the pharmaceutical composition of the invention includes a bacterial strain that results in a reduction of bacterial penetration or bacterial load in the mucus. In some embodiments, the pharmaceutical composition of the invention includes a bacterial strain that reduces sulfate-reducing bacteria (SRB) in a subject.

Additional criteria that may be utilized for selecting a bacterial strain for inclusion in the pharmaceutical composition of the invention include, but are not limited to, the ability of the bacterial strain to inhibit IgA-degrading bacteria, the ability of the bacterial strain to inhibit serotonin-producing and serotonin-inducing bacteria, the ability of the bacterial strain to enhance tryptophan availability, the ability of the bacterial strain to produce anti-inflammatory zwitterionic polysaccharides, modification of signaling molecules interacting with the Aryl Hydrocarbon Receptor, and/or the ability of the bacterial strain to block the vitamin D receptor (VCD) or vitamin D signaling.

In some embodiments, the pharmaceutical composition of the invention comprises a bacterial strain derived from any one of the phylum, class, order, family, genus, and/or species listed in Table 1.

In some embodiments, the pharmaceutical composition of the invention comprises a bacterial strain belonging to the phylum Bacteroidetes or Firmicutes. In exemplary embodiments, the pharmaceutical composition of the invention comprises a bacterial strain belonging to the class Clostridia, Bacteroidia, or Bacilli. In exemplary embodiments, the pharmaceutical composition of the invention comprises a bacterial strain belonging to the order Bacteroidales, Clostridiales, or Lactobacillales. In exemplary embodiments, the pharmaceutical composition of the invention comprises a bacterial strain belonging to the family Bacteroidaceae, Ruminococcaceae, Lachnospiraceae, or Streptococcacea. In exemplary embodiments, the pharmaceutical composition of the invention comprises a bacterial strain belonging to the genus Bacteroides, Blautia, Faecalibacterium, Coprococcus, Roseburia, Dorea, or Streptococcus. In exemplary embodiments, the pharmaceutical composition of the invention comprises a bacterial strain belonging to the species B. uniformis, F. prausnitzii, or E. faecis.

In various embodiments, individual bacterial strains are initially selected from Table 1 and subsequently pooled to form a mixture of bacterial strains. For example, in an embodiment, a mixture of bacterial strains may be formed by including one or more strains that has a 16S rRNA sequence that is at least about 97% identical with the 16S rRNA sequence of any one of the operational taxonomic units provided in Table 1.

Bacterial Strains

The present invention relates to pharmaceutical compositions (formulated for targeted delivery to the colon) of mixtures of bacterial strains that are introduced into the gut to replace the dysbiotic gut microbiome with a healthy community, reduce bile duct inflammation, and/or improve liver function in a patient with PSC, thereby preventing and/or treating PSC.

In embodiments, a mixture of bacterial strains useful in the present invention is contained in feces from a donor who satisfies at least one selection criterion, as described herein.

In some embodiments, a mixture of bacterial strains useful in the present invention is obtained by combining a plurality of isolated, purified, and/or cultured bacterial strains that are known to be present in feces of donors who satisfy at least one selection criterion, as described herein.

In embodiments, a mixture of bacterial strains useful in the present invention is contained in feces from a donor who satisfies at least one selection criterion, as described herein, is combined with a plurality of isolated, purified, and/or cultured bacterial strains that are known to be present in feces of donors who satisfy at least one selection criterion, as described herein.

The mixture of bacterial strains of the present invention can be delivered to patients in a variety of ways including orally (e.g., in a capsule), via ND/NG tube, or colonoscopically.

The mixture can also be formulated in a multitude of formulations including pure and/or isolated cultures, both lyophilized bacteria and aqueous solutions, spores, and as part of a broader community or mixtureconsortium of bacteria (e.g., a mixture of natural communities, including bacteria contained in a source material).

In various embodiments, the bacterial strains of the invention comprise bacteria isolated or purified from one or more humans, e.g., who satisfy at least one selection criterion, as described herein. In various embodiments, the present mixtures of bacterial strains is isolated or purified from one or more humans. For instance, the isolation or purification may be from feces of the one or more humans. Further, the isolation or purification may be from aspirates of the fluid in the GI tract or mucosal biopsies from a site in the GI tract.

In various embodiments, the bacterial strains of the invention are isolated or purified from its source material, i.e., separated from at least some of the components with which they were associated when initially produced (e.g., nature (from feces) or in an experimental setting (a laboratory stock) and/or produced, prepared, purified, and/or manufactured by man. Bacterial strains may be separated from at least about 10%, or about 20%, or about 30%, or about 40%, or about 50%, or about 60%, or about 70%, or about 80%, or about 90%, or more of the other components with which they were initially associated. In some embodiments, bacterial strains are more than about 80%, or about 85%, or about 90%, or about 91%, or about 92%, or about 93%, or about 94%, or about 95%, or about 96%, or about 97%, or about 98%, or about 99%, or more than about 99% pure.

In embodiments, bacterial strains for a bacterial mixture are directly obtained from human feces. In these embodiments, fecal matter is collected from one or more humans and processed ultimately until a formulation suitable for oral delivery and/or delivery into the GI tract is prepared.

In some embodiments, the bacterial strains are contained in fresh, frozen, dried, or reconstituted fecal material from a donor who satisfies at least one selection criterion, as described herein.

In some embodiments, the bacterial strains are from fresh, frozen, dried, or reconstituted fecal material, e.g., from a donor who satisfies at least one selection criterion, as described herein.

In some embodiments, the bacterial strains are contained in minimally processed fecal material from a donor who satisfies at least one selection criterion, as described herein.

In some embodiments, the bacterial strains are from minimally processed fecal material, e.g., from a donor who satisfies at least one selection criterion, as described herein.

In other embodiments, bacterial strains for a bacterial mixture are indirectly obtained from human feces and/or are obtained independent of human feces (e.g., from a bacterial cell bank or from a laboratory stock). When indirectly obtained, bacterial strains from human feces are cultured and the bacteria are expanded and then isolated and/or purified. The isolated/purified bacteria can be introduced into a bacterial mixture comprising bacterial strains directly obtained from human feces. Alternately, a plurality of isolated/purified bacteria can be combined into a defined bacterial mixture comprising only bacterial strains indirectly obtained from human feces or obtained independent of human feces.

In some embodiments, the bacteria are live, vegetative cells. In some embodiments, the bacteria are capable of forming spores. In some embodiments, the bacteria are in the form of spores, e.g., viable spores. In some embodiments, the mixtures of bacterial strains as described herein comprise live, vegetative cells and spores. In some embodiments, the mixture of bacterial strains as described herein is substantially free of live, vegetative cells. In some embodiments, the mixture of bacterial strains as described herein is substantially free of spores. In some embodiments, the bacterial strains are in the form of live, vegetative cells. In some embodiments, the bacterial strains are in the form of spores. In some embodiments, the bacterial strains are in the form of lyophilized cells. In some embodiments, the bacterial mixture comprises one or more of live, vegetative cells; spores; and lyophilized cells.

In some embodiments, the bacterial strains are non-pathogenic. For instance, in some embodiments, the bacterial strains are substantially free of organisms or entities which are capable of causing or affecting a disease, disorder or condition of a host organism containing the organism or entity. Illustrative pathogenic bacteria are provided elsewhere herein.

Illustrative pathogenic bacteria include C. difficile, Salmonella spp., enteropathogenic E. coli, multi-drug resistant bacteria such as Klebsiella, and E. coli, Carbapenem-resistent Enterobacteriaceae (CRE), extended spectrum beta-lactam resistant Enterococci (ESBL), fluoroquinolone-resistant Enterobacteriaceae, and vancomycin-resistant Enterococci (VRE). Further illustrative bacteria include Yersinia, Vibrio, Treponema, Streptococcus, Staphylococcus, Shigella, Salmonella, Rickettsia, Orientia, Pseudomonas, Neisseria, Mycoplasma, Mycobacterium, Listeria, Leptospira, Legionella, Klebsiella, Helicobacter, Haemophilus, Francisella, Escherichia, Ehrlichia, Enterococcus, Coxiella, Corynebacterium, Clostridium, Chlamydia, Chlamydophila, Campylobacter, Burkholderia, Brucella, Borrelia, Bordetella, Bifidobacterium, Bacillus, Proteus, Morganella, multi-drug resistant bacteria, extended spectrum beta-lactam resistant Enterococci (ESBL), Carbapenem-resistent Enterobacteriaceae (CRE), fluoroquinolone-resistant Enterobacteriaceae, and vancomycin-resistant Enterococci (VRE). Illustrative pathogenic bacteria include Aeromonas hydrophila, Campylobacter fetus, Plesiomonas shigelloides, Bacillus cereus, Campylobacter jejuni, Clostridium botulinum, Clostridium difficile, Clostridium perfringens, enteroaggregative Escherichia coli, enterohemorrhagic Escherichia coli, enteroinvasive Escherichia coli, enterotoxigenic Escherichia coli (such as, but not limited to, LT and/or ST), Escherichia coli 0157: H7, Helicobacter pylori, Klebsiellia pneumonia, Lysteria monocytogenes, Plesiomonas shigelloides, Salmonella spp., Salmonella typhi, Salmonella paratyphi, Shigella spp., Staphylococcus spp., Staphylococcus aureus, vancomycin-resistant enterococcus spp., Vibrio spp., Vibrio cholerae, Vibrio parahaemolyticus, Vibrio vulnificus, and Yersinia enterocolitica. Specifically-relevant pathogenic bacteria include Antibiotic-resistant Proteobacteria, Vancomycin Resistant Enterococcus (VRE), Carbapenem Resistant Enterobacteriaceae (CRE), fluoroquinolone-resistant Enterobacteriaceae, and Extended Spectrum Beta-Lactamase producing Enterobacteriaceae (ESBL-E).

In various embodiments, a pharmaceutical composition of the invention comprises one or more bacterial strains, e.g., isolated, purified, and/or cultured bacterial strains, having a 16S rRNA sequence that is at least about 80% identical to the 16S rRNA sequence of any one of the operational taxonomic units (OTUs) provided in Table 1. For example, the pharmaceutical composition may comprise one or more bacterial strains having a 16S rRNA sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical with the 16S rRNA sequence of any one of the OTUs provided in Table 1. In an embodiment, the pharmaceutical composition may comprise one or more bacterial strains having a 16S rRNA sequence that is at least about 97%, at least about 98%, at least about 99%, or about 100% identical with the 16S rRNA sequence of any one of the OTUs provided in Table 1.

In various embodiments, a pharmaceutical composition of the invention incudes fecal material from a donor who satisfies at least one selection criterion, as described herein, and which comprises one or more bacterial strains having a 16S rRNA sequence that is at least about 80% identical to the 16S rRNA sequence of any one of the OTUs provided in Table 1. For example, the fecal material may comprise one or more bacterial strains having a 16S rRNA sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical with the 16S rRNA sequence of any one of the OTUs provided in Table 1. In an embodiment, the fecal matter may comprise one or more bacterial strains having a 16S rRNA sequence that is at least about 97%, at least about 98%, at least about 99%, or about 100% identical with the 16S rRNA sequence of any one of the OTUs provided in Table 1.

In various embodiments, the pharmaceutical composition of the invention comprises a bacterial mixture of at least about 50 different bacterial strains, or at least about 49 different bacterial strains, or at least about 48 different bacterial strains, or at least about 47 different bacterial strains, or at least about 46 different bacterial strains, or at least about 45 different bacterial strains, or at least about 44 different bacterial strains, or at least about 43 different bacterial strains, or at least about 42 different bacterial strains, or at least about 41 different bacterial strains, or at least about 40 different bacterial strains, or at least about 39 bacterial strains, or at least about 38 bacterial strains, or at least about 37 bacterial strains, or at least about 36 bacterial strains, or at least about 35 bacterial strains, or at least about 34 bacterial strains, or at least about 33 bacterial strains, or at least about 32 bacterial strains, or at least about 31 bacterial strains, or at least about 30 bacterial strains, or at least about 29 bacterial strains, or at least about 28 bacterial strains, or at least about 27 bacterial strains, or at least about 26 bacterial strains, or at least about 25 bacterial strains, or at least about 24 bacterial strains, or at least about 23 bacterial strains, or at least about 22 bacterial strains, or at least about 21 bacterial strains, or at least about 20 bacterial strains, or at least about 19 bacterial strains, or at least about 18 bacterial strains, or at least about 17 bacterial strains, or at least about 16 bacterial strains, or at least about 15 bacterial strains, or at least about 14 bacterial strains, or at least about 13 bacterial strains, or at least about 12 bacterial strains, or at least about 11 bacterial strains, or at least about 10 bacterial strains, or at least about 9 bacterial strains, or at least about 8 bacterial strains, or at least about 7 bacterial strains, or at least about 6 bacterial strains, or at least about 5 bacterial strains, or at least about 4 bacterial strains, or at least about 3 bacterial strains, or at least about 2 bacterial strains, or about 1 bacterial strain with reference to Table 1, e.g., as listed in Table 1 or having a 16S rRNA sequence that is, as examples, at least about 95%, at least about 96%, at least about 97%, at least about 98%, at least about 99%, or about 100% identical with the 16S rRNA sequence of any one of the strains listed in Table 1.

In various embodiments, the pharmaceutical composition of the invention comprises a bacterial mixture of about 50 or fewer different bacterial strains as described herein (e.g., with reference to Table 1).

In some embodiments, the pharmaceutical composition of the invention comprises greater than about 2, greater than about 5, or greater than about 10, or greater than about 15, or greater than about 20, or greater than about 25, or greater than about 30, or greater than about 35, or greater than about 40, or greater than about 45, or greater than about 50, greater than about 75, or greater than about 100 different bacterial strains as described herein (e.g., with reference to Table 1).

In some embodiments, the pharmaceutical composition of the invention comprises less than about 5, or less than about 10, or less than about 15, or less than about 20, or less than about 25, or less than about 30, or less than about 35, or less than about 40, or less than about 45, or less than about 50 different bacterial strains as described herein (e.g., with reference to Table 1).

In some embodiments, the pharmaceutical composition of the invention comprises about 10 to about 50 different bacterial strains as described herein (e.g., with reference to Table 1), including about 10 to about 45, or about 10 to about 40, or about 10 to about 30, or about 10 to about 20, or about 10 to about 15 different bacterial strains.

In some embodiments, the pharmaceutical composition of the invention comprises about 10 to about 20 different bacterial strains as described herein (e.g., with reference to Table 1).

In various embodiments, the mixtures of bacterial strains are selected from any of the bacterial strains listed in Table 1 below or the bacterial strains having a 16S rRNA sequence that is, as examples, at least about 95%, at least about 96%, at least about 97%, at least about 98%, at least about 99%, or about 100% identical with the 16S rRNA sequence of any one of the strains listed in Table 1 below.

In various embodiments, the mixtures of bacterial strains lack or have a low abundance of bacteria that are specifically found in fecal material originating from a PSC patient, e.g., bacteria known to be in or found in fecal material from a PSC patient and not in fecal material from a healthy person (who does not have PSC).

In various embodiments, the mixtures of bacterial strains comprise one or more (e.g., one, two, three, four, five, six, seven, eight, nine, ten, eleven, twelve, or more, and up to all thirty-two) strains from the priority bacterial strains in Table 1 which are identifiable as having a 16S V4 sequence of one of SEQ ID NO: 1 to SEQ ID NO: 32.

In various embodiments, the mixtures of bacterial strains comprise one or more (e.g., one, two, three, four, five, six, seven, eight, nine, ten, eleven, or twelve) strains from the priority clusters in Table 1 which are identifiable as having a 16S V4 sequence that is at least 97% identical to one of SEQ ID NO: 10, SEQ ID NO: 13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240.

In various embodiments, at least one (e.g., at least about five, at least about ten, at least about fifteen, at least about twenty, at least about twenty-five, at least about thirty, and about thirty-five) bacterial strain included in the bacterial mixture comprises a 16S V4 sequence that is greater than about 97% identical (e.g., about identical) to one of SEQ ID NO: 1 to SEQ ID NO: 32.

In various embodiments, the mixtures of bacterial strains comprises one or more (e.g., one, two, three, four, five, six, seven, eight, nine, ten, eleven, or twelve) of the following bacterial strains: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

In various embodiments, the mixtures of bacterial strains in a pharmaceutical composition may stimulate and/or activate Toll-like receptor activity (e.g., TLR1, and/or TLR2, and/or TLR3, and/or TLR4, and/or TLR5, and/or TLR6, and/or TLR7, and/or TLR8, and/or TLR9, and/or TLR10, and/or TLR11, and/or TLR12, and/or TLR13).

In some embodiments, the mixtures of bacterial strains in a pharmaceutical composition treat or prevent the various GI disorders disclosed herein and/or as known in the art to be a result of gut dysbiosis.

In various embodiments, the mixture of bacterial strains in a pharmaceutical composition includes one or more bacterial strains that interact synergistically for treating or preventing PSC.

In some embodiments, the mixtures of bacterial strains in a pharmaceutical composition reduce, ameliorate, or eliminate one or more symptom(s) associated with PSC,

Table 1 contains 268 strains (OTUs) with information derived from an FMT study and analysis of donors. OTUs were defined by a unique 16S V4 sequence found in at least two separate samples in the study (SEQ ID Nos identified in Column R). The 268 OTUs in Table 1 were selected by requiring engraftment in at least one PSC patient (Column E>0) and an association with ALP decrease (Column L<0). OTUs are sorted by frequency of engraftment (Column E).
Columns:
A. Taxonomic classification (Kingdom, Phylum, Class, Order, Family, Genus)
B. Prevalence of strain among thirty-one healthy donors
C. Mean relative abundance of strain among thirty-one healthy donors
D. Coefficient of variation (CV) of relative strain abundance among thirty-one healthy donors; higher CV indicates greater variability among donors
E. Frequency strain was observed engrafting in PSC patients in an FMT study; higher frequency indicates greater probability strain engrafts in a given patient
F. Spearman correlation coefficient between strain abundance in patients and patients' level of serum alkaline phosphatase; negative correlation indicates association with slower disease progression
G. Pearson correlation coefficient between strain abundance in patients and patients' level of serum alkaline phosphatase; negative correlation indicates association with slower disease progression
H. Spearman correlation coefficient between strain abundance in patients and patients' level of serum alanine aminotransferase; negative correlation indicates association with slower disease progression
I. Pearson correlation coefficient between strain abundance in patients and patients' level of serum alanine aminotransferase; negative correlation indicates association with slower disease progression
J. Spearman correlation coefficient between strain abundance in patients and patients' level of serum aspartate aminotransferase; negative correlation indicates association with slower disease progression
K. Pearson correlation coefficient between strain abundance in patients and patients' level of serum aspartate aminotransferase; negative correlation indicates association with slower disease progression
L. Spearman correlation coefficient between strain abundance in patients and patients' level of serum alkaline phosphatase relative to baseline; negative correlation indicates association with improved liver function
M. Pearson correlation coefficient between strain abundance in patients and patients' level of serum alkaline phosphatase relative to baseline; negative correlation indicates association with improved liver function
N. Spearman correlation coefficient between strain abundance in patients and patients' level of serum aspartate aminotransferase relative to baseline; negative correlation indicates association with improved liver function
O. Pearson correlation coefficient between strain abundance in patients and patients' level of serum aspartate aminotransferase relative to baseline; negative correlation indicates association with improved liver function
P. Spearman correlation coefficient between strain abundance in patients and patients' level of serum alanine aminotransferase relative to baseline; negative correlation indicates association with improved liver function
Q. Pearson correlation coefficient between strain abundance in patients and patients' level of serum alanine aminotransferase relative to baseline; negative correlation indicates association with improved liver function
R. Sequence Identifier (i.e., SEQ ID NO) for its 16S V4 sequence

TABLE 1
Exemplary OTUs Useful in the Present Invention

| A | B | C | D | E | F | G | H | I | J | K | L | M | N | O | P | Q | R |
|---|---|---|---|---|---|---|---|---|---|---|---|---|---|---|---|---|---|
| Bacteria;Actinobacteria; | 0.387097 | 0.002043 | 2.632139 | 0.5 | −0.34977 | −0.227 | −0.25351 | −0.12257 | −0.36947 | −0.28135 | −0.34519 | −0.17333 | −0.27825 | −0.11002 | −0.45112 | −0.24369 | 1 |
| Actinobacteria; | | | | | | | | | | | | | | | | | |
| Bifidobacteriales; | | | | | | | | | | | | | | | | | |
| Bifidobacteriaceae; | | | | | | | | | | | | | | | | | |
| Bifidobacterium | | | | | | | | | | | | | | | | | |
| Bacteria;Actinobacteria; | 0.064516 | 2.58E−05 | 4.006938 | 0.125 | 0.006933 | −0.03062 | 0.035288 | −0.07385 | −0.10688 | −0.09239 | −0.28648 | −0.16677 | −0.14974 | −0.11698 | −0.103 | −0.19152 | 2 |
| Actinobacteria; | | | | | | | | | | | | | | | | | |
| Bifidobacteriales; | | | | | | | | | | | | | | | | | |
| Bifidobacteriaceae; | | | | | | | | | | | | | | | | | |
| Bifidobacterium | | | | | | | | | | | | | | | | | |
| Bacteria;Bacteroidetes; | 0.419355 | 5.89E−05 | 1.533308 | 0.375 | 0.139081 | 0.126417 | 0.034642 | 0.045797 | 0.038063 | 0.096624 | −0.26646 | −0.21559 | −0.23119 | −0.2648 | −0.21689 | −0.33545 | 3 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.516129 | 0.000112 | 1.28406 | 0.25 | 0.010978 | 0.093039 | 0.174708 | 0.246684 | 0.011237 | 0.221091 | −0.42187 | −0.20146 | −0.21382 | −0.16327 | −0.24053 | −0.19112 | 4 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.354839 | 2.85E−05 | 1.601001 | 0.125 | 0.047213 | −0.03339 | 0.003464 | −0.19605 | 0.047659 | −0.02841 | −0.37755 | −0.26166 | −0.28821 | −0.30963 | −0.3393 | −0.55277 | 5 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.387097 | 4.48E−05 | 1.955845 | 0.125 | −0.20812 | −0.16583 | −0.1213 | −0.2299 | −0.18678 | −0.17149 | −0.19467 | −0.15989 | −0.21636 | −0.36 | −0.29225 | −0.48979 | 6 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.419355 | 4.87E−05 | 1.791645 | 0.125 | 0.022131 | −0.02334 | −0.05555 | −0.18851 | −0.06411 | −0.08781 | −0.29211 | −0.20076 | −0.24175 | −0.29866 | −0.41638 | −0.52497 | 7 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.516129 | 7.68E−05 | 1.525709 | 0.125 | 0.155338 | 0.432029 | 0.136867 | 0.483215 | 0.179187 | 0.531582 | −0.33937 | −0.12167 | −0.17868 | −0.10005 | −0.31234 | −0.15485 | 8 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.387097 | 4.72E−05 | 1.496377 | 0.125 | −0.01851 | −0.02094 | 0.004628 | −0.13508 | −0.10647 | −0.07438 | −0.18991 | −0.19184 | −0.35261 | −0.39104 | −0.28162 | −0.44756 | 9 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Firmicutes; | 0.709677 | 0.003001 | 1.5855 | 0.25 | −0.25935 | −0.37566 | −0.36884 | −0.4035 | −0.43927 | −0.37264 | −0.34082 | −0.33961 | −0.20006 | −0.29713 | −0.48947 | −0.60446 | 10 |
| Bacilli; | | | | | | | | | | | | | | | | | |
| Lactobacillales; | | | | | | | | | | | | | | | | | |
| Lactobacillaceae; | | | | | | | | | | | | | | | | | |
| Lactobacillus | | | | | | | | | | | | | | | | | |
| Bacteria;Firmicutes; | 0.354839 | 4.27E−05 | 1.864112 | 0.125 | −0.29504 | −0.21219 | −0.22578 | −0.11235 | −0.30823 | −0.23633 | −0.35778 | −0.22013 | −0.1439 | −0.16069 | −0.44572 | −0.28612 | 11 |
| Bacilli; | | | | | | | | | | | | | | | | | |
| Lactobacillales; | | | | | | | | | | | | | | | | | |
| Lactobacillaceae; | | | | | | | | | | | | | | | | | |
| Lactobacillus | | | | | | | | | | | | | | | | | |
| Bacteria;Firmicutes; | 0.193548 | 3.15E−05 | 2.693763 | 0.125 | −0.30911 | −0.2351 | −0.15959 | −0.15444 | −0.35277 | −0.2077 | −0.26881 | −0.20936 | −0.28381 | −0.25023 | −0.30255 | −0.26439 | 12 |
| Bacilli; | | | | | | | | | | | | | | | | | |
| Lactobacillales; | | | | | | | | | | | | | | | | | |
| Lactobacillaceae; | | | | | | | | | | | | | | | | | |
| unclassified | | | | | | | | | | | | | | | | | |
| Bacteria;Firmicutes; | 0.967742 | 0.023298 | 0.949369 | 0.375 | −0.23603 | −0.2366 | −0.11571 | −0.27867 | −0.10814 | −0.1751 | −0.23666 | −0.17192 | −0.15231 | −0.19288 | −0.30284 | −0.36761 | 13 |
| Clostridia; | | | | | | | | | | | | | | | | | |
| Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.451613 | 9.73E−05 | 1.7581 | 0.25 | −0.33554 | −0.27909 | −0.21857 | −0.15187 | −0.26317 | −0.20636 | −0.42204 | −0.20614 | −0.28489 | −0.13675 | −0.40093 | −0.21851 | 14 |
| Clostridia; | | | | | | | | | | | | | | | | | |
| Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.645161 | 0.000108 | 1.302277 | 0.125 | −0.08539 | −0.03846 | −0.032801 | −0.04747 | −0.06779 | −0.1244 | −0.15627 | −0.16342 | −0.39133 | −0.38234 | −0.28393 | −0.2565 | 15 |
| Clostridia; | | | | | | | | | | | | | | | | | |
| Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.516129 | 6.89E−05 | 1.44225 | 0.125 | −0.21316 | −0.28801 | −0.23035 | −0.31198 | −0.19329 | −0.20327 | −0.18986 | −0.20118 | −0.17146 | −0.11483 | −0.19511 | −0.25447 | 16 |
| Clostridia; | | | | | | | | | | | | | | | | | |
| Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.483871 | 5.59E−05 | 1.424557 | 0.125 | −0.15242 | −0.18325 | −0.15724 | −0.27361 | −0.12797 | −0.19999 | −0.35611 | −0.222 | −0.14982 | −0.18786 | −0.26272 | −0.36236 | 17 |
| Clostridia; | | | | | | | | | | | | | | | | | |
| Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.451613 | 4.11E−05 | 1.420253 | 0.125 | 0.023573 | −0.07402 | 0.004917 | −0.16858 | 0.000615 | −0.05532 | −0.29507 | −0.20216 | −0.17027 | −0.1271 | −0.20488 | −0.31912 | 18 |
| Clostridia; | | | | | | | | | | | | | | | | | |
| Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.516129 | 4.81E−05 | 1.332431 | 0.125 | −0.05457 | −0.13243 | −0.12423 | −0.20889 | −0.1361 | −0.16782 | −0.20652 | −0.1012 | −0.22419 | −0.31286 | −0.19212 | −0.35199 | 19 |
| Clostridia; | | | | | | | | | | | | | | | | | |
| Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.322581 | 3.50E−05 | 1.680503 | 0.125 | 0.08749 | 0.051147 | −0.0888 | −0.13044 | −0.15877 | −0.05872 | −0.19598 | −0.19582 | −0.29454 | −0.32053 | −0.29335 | −0.43883 | 20 |
| Clostridia; | | | | | | | | | | | | | | | | | |
| Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.935484 | 0.019302 | 0.964755 | 0.25 | 0.157295 | 0.178245 | 0.076129 | 0.129087 | 0.055997 | 0.209291 | −0.14522 | −0.14343 | −0.19245 | −0.14569 | −0.33807 | −0.27198 | 21 |
| Clostridia;Clostridiales; | | | | | | | | | | | | | | | | | |
| Ruminococcaceae; | | | | | | | | | | | | | | | | | |
| Faecalibacterium | | | | | | | | | | | | | | | | | |
| Bacteria;Firmicutes; | 0.419355 | 3.04E−05 | 1.403661 | 0.125 | 0.119766 | 0.31462 | 0.076206 | 0.307596 | 0.170277 | 0.375301 | −0.33014 | −0.14481 | −0.19422 | −0.15413 | −0.37157 | −0.23412 | 22 |
| Clostridia;Clostridiales; | | | | | | | | | | | | | | | | | |
| Ruminococcaceae; | | | | | | | | | | | | | | | | | |
| Faecalibacterium | | | | | | | | | | | | | | | | | |
| Bacteria;Firmicutes; | 0.387097 | 3.15E−05 | 1.718051 | 0.125 | 0.096796 | 0.240273 | 0.116152 | 0.249041 | 0.027803 | 0.341002 | −0.31088 | −0.22895 | −0.33578 | −0.17746 | −0.24489 | −0.24518 | 23 |
| Clostridia;Clostridiales; | | | | | | | | | | | | | | | | | |
| Ruminococcaceae; | | | | | | | | | | | | | | | | | |
| Faecalibacterium | | | | | | | | | | | | | | | | | |
| Bacteria;Firmicutes; | 0.548387 | 0.000153 | 1.528122 | 0.125 | 0.116321 | 0.017476 | 0.130711 | −0.04601 | 0.025393 | −0.08102 | −0.25454 | −0.13472 | −0.21724 | −0.17059 | −0.25693 | −0.14601 | 24 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.322581 | 0.000128 | 1.968194 | 0.125 | −0.15004 | −0.19119 | −0.24008 | −0.27165 | −0.27045 | −0.22088 | −0.30215 | −0.15548 | −0.26159 | −0.23863 | −0.39494 | −0.41398 | 25 |
| Clostridia; | | | | | | | | | | | | | | | | | |
| Clostridiales;unclassified; | | | | | | | | | | | | | | | | | |
| unclassified | | | | | | | | | | | | | | | | | |
| Bacteria;Firmicutes; | 0.483871 | 0.000357 | 1.341812 | 0.375 | −0.42507 | −0.28832 | −0.58959 | −0.38243 | −0.54754 | −0.30992 | −0.1754 | −0.24942 | −0.3376 | −0.21576 | −0.52688 | −0.57734 | 26 |
| Clostridia; | | | | | | | | | | | | | | | | | |
| unclassified;unclassified; | | | | | | | | | | | | | | | | | |
| unclassified | | | | | | | | | | | | | | | | | |
| Bacteria;Firmicutes; | 0.548387 | 0.000268 | 1.448045 | 0.25 | −0.07087 | −0.02405 | −0.06605 | −0.1442 | −0.05405 | −0.05217 | −0.26001 | −0.16859 | −0.17139 | −0.21998 | −0.4122 | −0.49904 | 27 |
| Clostridia; | | | | | | | | | | | | | | | | | |
| unclassified;unclassified; | | | | | | | | | | | | | | | | | |
| unclassified | | | | | | | | | | | | | | | | | |
| Bacteria;Firmicutes; | 0.032258 | 9.03E−06 | 5.567764 | 0.125 | −0.14029 | −0.19381 | −0.25475 | −0.24414 | −0.15193 | −0.18812 | −0.15531 | −0.16394 | −0.15451 | −0.1323 | −0.36423 | −0.41803 | 28 |
| Erysipelotrichia; | | | | | | | | | | | | | | | | | |
| Erysipelotrichales; | | | | | | | | | | | | | | | | | |
| Erysipelotrichaceae; | | | | | | | | | | | | | | | | | |
| Erysipelotrichaceae_ | | | | | | | | | | | | | | | | | |
| incertae_sedis | | | | | | | | | | | | | | | | | |
| Bacteria;Firmicutes; | 0.032258 | 2.17E−05 | 5.567764 | 0.375 | −0.16802 | −0.1063 | −0.21666 | −0.27511 | −0.03689 | −0.09347 | −0.29366 | −0.18023 | −0.22528 | −0.18536 | −0.39877 | −0.45399 | 29 |
| Erysipelotrichia; | | | | | | | | | | | | | | | | | |
| Erysipelotrichales; | | | | | | | | | | | | | | | | | |
| Erysipelotrichaceae; | | | | | | | | | | | | | | | | | |
| unclassified | | | | | | | | | | | | | | | | | |
| Bacteria;Firmicutes; | 0.032258 | 5.42E−06 | 5.567764 | 0.125 | 0.018933 | 0.025605 | −0.0274 | −0.04615 | −0.00947 | −0.07576 | −0.2156 | −0.13789 | −0.10154 | −0.12915 | −0.12873 | −0.10687 | 30 |
| Erysipelotrichia; | | | | | | | | | | | | | | | | | |
| Erysipelotrichales; | | | | | | | | | | | | | | | | | |
| Erysipelotrichaceae; | | | | | | | | | | | | | | | | | |
| unclassified | | | | | | | | | | | | | | | | | |
| Bacteria;Proteobacteria; | 0.032258 | 1.81E−05 | 5.567764 | 0.75 | −0.30166 | −0.3019 | −0.33396 | −0.37857 | −0.11096 | −0.21753 | −0.31702 | −0.12079 | −0.14055 | −0.18729 | −0.4038 | −0.35905 | 31 |
| Deltaproteobacteria; | | | | | | | | | | | | | | | | | |
| Desulfovibrionales; | | | | | | | | | | | | | | | | | |
| Desulfovibrionaceae; | | | | | | | | | | | | | | | | | |
| Bilophila | | | | | | | | | | | | | | | | | |
| Bacteria;Proteobacteria; | 0.096774 | 0.000417 | 5.175615 | 0.125 | −0.24808 | −0.05931 | −0.21954 | −0.11409 | −0.18073 | −0.02756 | −0.27413 | −0.22295 | −0.15451 | −0.10576 | −0.45063 | −0.44027 | 32 |
| unclassified;unclassified; | | | | | | | | | | | | | | | | | |
| unclassified;unclassified | | | | | | | | | | | | | | | | | |
| Bacteria;Actinobacteria; | 0.032258 | 3.61E−06 | 5.567764 | 0.125 | −0.20953 | −0.01571 | −0.14218 | −0.0174 | −0.30766 | −0.0617 | −0.23709 | −0.11461 | −0.11443 | −0.01314 | −0.21049 | −0.12189 | 33 |
| Actinobacteria; | | | | | | | | | | | | | | | | | |
| Bifidobacteriales; | | | | | | | | | | | | | | | | | |
| Bifidobacteriaceae; | | | | | | | | | | | | | | | | | |
| Bifidobacterium | | | | | | | | | | | | | | | | | |
| Bacteria;Actinobacteria; | 0.193548 | 3.20E−05 | 2.601326 | 0.125 | 0.13822 | −0.14566 | 0.11203 | −0.18711 | 0.052646 | −0.1234 | −0.10611 | −0.08761 | −0.21566 | −0.04346 | −0.16603 | −0.1818 | 34 |
| Actinobacteria; | | | | | | | | | | | | | | | | | |
| Bifidobacteriales; | | | | | | | | | | | | | | | | | |
| Bifidobacteriaceae; | | | | | | | | | | | | | | | | | |
| Bifidobacterium | | | | | | | | | | | | | | | | | |
| Bacteria;Actinobacteria; | 0.032258 | 3.61E−06 | 5.567764 | 0.125 | −0.03481 | 0.005149 | 0.072542 | 0.042969 | −0.04354 | −0.03265 | −0.03341 | −0.05851 | 0.136273 | −0.01509 | 0.012353 | 0.010704 | 35 |
| Actinobacteria; | | | | | | | | | | | | | | | | | |
| Bifidobacteriales; | | | | | | | | | | | | | | | | | |
| Bifidobacteriaceae; | | | | | | | | | | | | | | | | | |
| Bifidobacterium | | | | | | | | | | | | | | | | | |
| Bacteria;Actinobacteria; | 0.483871 | 8.55E−05 | 1.736353 | 0.125 | 0.017218 | −0.08058 | 0.098266 | −0.02963 | 0.014857 | −0.06752 | −0.26765 | −0.15548 | −0.2464 | −0.13579 | −0.03863 | −0.06409 | 36 |
| Actinobacteria; | | | | | | | | | | | | | | | | | |
| Bifidobacteriales; | | | | | | | | | | | | | | | | | |
| Bifidobacteriaceae; | | | | | | | | | | | | | | | | | |
| Bifidobacterium | | | | | | | | | | | | | | | | | |
| Bacteria;Actinobacteria; | 0.870968 | 0.007308 | 1.629436 | 0.125 | 0.240486 | 0.183119 | 0.185048 | 0.203408 | 0.202005 | 0.174039 | −0.15683 | −0.11806 | −0.36256 | −0.1719 | −0.27865 | −0.03814 | 37 |
| Actinobacteria; | | | | | | | | | | | | | | | | | |
| Bifidobacteriales; | | | | | | | | | | | | | | | | | |
| Bifidobacteriaceae; | | | | | | | | | | | | | | | | | |
| Bifidobacterium | | | | | | | | | | | | | | | | | |
| Bacteria;Actinobacteria; | 0.129032 | 3.11E−05 | 4.847167 | 0.125 | −0.04268 | −0.11975 | 0.049059 | −0.11551 | 0.20867 | 0.026336 | −0.18863 | −0.01517 | 0.129979 | 0.133684 | −0.09168 | −0.07899 | 38 |
| Actinobacteria; | | | | | | | | | | | | | | | | | |
| Bifidobacteriales; | | | | | | | | | | | | | | | | | |
| Bifidobacteriaceae; | | | | | | | | | | | | | | | | | |
| Bifidobacterium | | | | | | | | | | | | | | | | | |
| Bacteria;Actinobacteria; | 0.806452 | 0.007674 | 1.090409 | 0.5 | −0.12492 | −0.13613 | −0.20483 | −0.11911 | −0.00243 | −0.05509 | −0.10674 | 0.104698 | −0.13883 | −0.0195 | −0.2976 | −0.02708 | 39 |
| Actinobacteria; | | | | | | | | | | | | | | | | | |
| Coriobacteriales; | | | | | | | | | | | | | | | | | |
| Coriobacteriaceae; | | | | | | | | | | | | | | | | | |
| Collinsella | | | | | | | | | | | | | | | | | |
| Bacteria;Actinobacteria; | 0.032258 | 1.63E−05 | 5.567764 | 0.375 | −0.39462 | −0.08363 | −0.35985 | 0.077851 | −0.32433 | −0.16207 | −0.06934 | 0.079825 | −0.08552 | 2.22E−05 | −0.34377 | 0.031549 | 40 |
| Actinobacteria; | | | | | | | | | | | | | | | | | |
| Coriobacteriales; | | | | | | | | | | | | | | | | | |
| Coriobacteriaceae; | | | | | | | | | | | | | | | | | |
| Collinsella | | | | | | | | | | | | | | | | | |
| Bacteria;Actinobacteria; | 0.129032 | 3.75E−05 | 3.234235 | 0.125 | −0.1845 | −0.17997 | −0.24425 | −0.19321 | 0.013003 | −0.02535 | −0.35511 | −0.15891 | −0.04232 | 0.019563 | −0.44672 | −0.21665 | 41 |
| Actinobacteria; | | | | | | | | | | | | | | | | | |
| Coriobacteriales; | | | | | | | | | | | | | | | | | |
| Coriobacteriaceae; | | | | | | | | | | | | | | | | | |
| Slackia | | | | | | | | | | | | | | | | | |
| Bacteria;Actinobacteria; | 0.032258 | 5.24E−05 | 5.567764 | 0.875 | −0.19348 | −0.08212 | −0.21885 | −0.12051 | 0.09771 | 0.065976 | −0.17235 | 0.167235 | 0.030288 | 0.124104 | −0.25316 | −0.06603 | 42 |
| Actinobacteria; | | | | | | | | | | | | | | | | | |
| Coriobacteriales; | | | | | | | | | | | | | | | | | |
| Coriobacteriaceae; | | | | | | | | | | | | | | | | | |
| unclassified | | | | | | | | | | | | | | | | | |
| Bacteria;Actinobacteria; | 0.580645 | 0.000148 | 1.576176 | 0.125 | 0.02917 | 0.056293 | −0.01628 | −0.01448 | 0.187988 | 0.094125 | −0.10593 | 0.084555 | −0.09837 | −0.03715 | −0.16355 | −0.05331 | 43 |
| Actinobacteria; | | | | | | | | | | | | | | | | | |
| Coriobacteriales; | | | | | | | | | | | | | | | | | |
| Coriobacteriaceae; | | | | | | | | | | | | | | | | | |
| unclassified | | | | | | | | | | | | | | | | | |
| Bacteria;Bacteroidetes; | 0.774194 | 0.007111 | 2.387753 | 1 | −0.33995 | −0.30101 | −0.40956 | −0.36995 | −0.21815 | −0.20104 | −0.04237 | 0.418045 | −0.09419 | −0.0862 | −0.337 | −0.31154 | 44 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.225806 | 0.001307 | 2.567831 | 0.875 | −0.26377 | −0.20848 | −0.39961 | −0.33833 | −0.23334 | −0.21613 | −0.086 | −0.00882 | −0.13225 | −0.07384 | −0.31964 | −0.39467 | 45 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.483871 | 0.007679 | 1.857098 | 0.875 | −0.08592 | −0.10673 | −0.16365 | −0.20612 | 0.090006 | 0.007941 | −0.10785 | 0.244459 | 0.126236 | 0.207516 | −0.13129 | −0.04003 | 46 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.451613 | 0.000193 | 2.0077 | 0.75 | −0.19161 | −0.25423 | −0.2589 | −0.30237 | −0.00653 | −0.09258 | −0.08195 | 0.243552 | −0.11612 | −0.13031 | −0.24214 | −0.21402 | 47 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.096774 | 0.000141 | 4.75886 | 0.75 | −0.42176 | −0.34513 | −0.48766 | −0.31973 | −0.27014 | −0.21743 | −0.08072 | 0.18805 | −0.02952 | −0.02707 | −0.27118 | −0.14877 | 48 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.354839 | 0.000258 | 2.936627 | 0.75 | −0.09788 | −0.08696 | −0.0828 | −0.09601 | 0.222924 | 0.10165 | −0.16018 | 0.264473 | 0.135557 | 0.209227 | −0.11332 | 0.070552 | 49 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.677419 | 0.00027 | 1.746128 | 0.5 | −0.2078 | −0.29241 | −0.17145 | −0.28838 | 0.069954 | −0.08683 | −0.04823 | 0.436448 | −0.01328 | −0.03837 | −0.18759 | −0.11595 | 50 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.612903 | 0.000269 | 1.354509 | 0.5 | −0.14736 | −0.03279 | −0.14244 | −0.06596 | 0.044371 | 0.014861 | −0.15129 | 0.428484 | −0.0655 | −0.09311 | −0.23587 | −0.09364 | 51 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.677419 | 0.000573 | 1.73257 | 0.5 | −0.20016 | −0.22648 | −0.14338 | −0.24537 | 0.096649 | −0.03568 | −0.11759 | 0.342245 | −0.02539 | −0.07576 | −0.26994 | −0.15777 | 52 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.709677 | 0.002944 | 1.47763 | 0.5 | −0.12837 | −0.21771 | −0.00152 | −0.08525 | 0.198501 | 0.01255 | −0.19699 | 0.057301 | 0.192682 | 0.095091 | 0.006228 | 0.107137 | 53 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.225806 | 8.70E−05 | 3.084331 | 0.375 | 0.000829 | −0.11058 | −0.01845 | −0.16632 | 0.221182 | 0.018391 | −0.10009 | 0.174952 | 0.10585 | 0.09471 | −0.03574 | −0.02991 | 54 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.645161 | 0.010934 | 1.595413 | 0.375 | 0.141242 | 0.189443 | 0.154548 | 0.180209 | 0.286707 | 0.20122 | −0.03683 | 0.122906 | 0.065217 | −0.0168 | 0.000153 | 0.047478 | 55 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.645161 | 0.000116 | 1.247905 | 0.375 | 0.04879 | 0.015597 | 0.154252 | 0.015501 | 0.100325 | 0.054454 | −0.103 | −0.05103 | −0.14039 | −0.15392 | −0.163 | −0.10341 | 56 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.774194 | 0.004669 | 2.395168 | 0.375 | 0.146819 | 0.329569 | 0.278927 | 0.498595 | 0.451033 | 0.466433 | −0.17965 | 0.007838 | 0.089622 | 0.01275 | −0.07776 | 0.108771 | 57 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.870968 | 0.006649 | 2.327538 | 0.375 | 0.17015 | 0.328318 | 0.168556 | 0.374953 | 0.392385 | 0.52352 | −0.12343 | −0.04328 | 0.21722 | 0.221506 | −0.02348 | 0.100106 | 58 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.483871 | 0.000103 | 1.477709 | 0.375 | 0.004016 | 0.031266 | 0.048511 | −0.01469 | 0.175487 | 0.049664 | −0.06379 | 0.58646 | −0.07353 | −0.10746 | −0.16518 | −0.01452 | 59 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 1 | 0.045374 | 1.056667 | 0.375 | −0.0496 | 0.08588 | 0.053854 | 0.167261 | 0.031389 | 0.156866 | −0.1607 | −0.04623 | −0.16398 | −0.15101 | −0.3108 | −0.3064 | 60 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.290323 | 6.61E−05 | 2.027389 | 0.375 | 0.006461 | −0.05503 | −0.05628 | −0.20865 | 0.14241 | −0.10776 | −0.15413 | 0.015581 | 0.144386 | −0.04674 | −0.08544 | −0.16654 | 61 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.290323 | 0.000105 | 2.679136 | 0.375 | −0.10777 | −0.14478 | −0.06156 | −0.09078 | 0.25757 | 0.12058 | −0.10114 | 0.403333 | 0.134985 | 0.24803 | −0.09374 | 0.149343 | 62 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.193548 | 2.29E−05 | 2.50837 | 0.25 | −0.00874 | 0.118561 | −0.12509 | −0.03857 | 0.015353 | 0.014784 | −0.10905 | −0.04259 | −0.03859 | 0.065417 | −0.16315 | −0.03914 | 63 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.83871 | 0.002628 | 1.137709 | 0.25 | −0.38401 | −0.10079 | −0.2058 | 0.098763 | −0.23562 | −0.16046 | −0.06823 | 0.059501 | 0.013102 | 0.01494 | −0.10442 | 0.093928 | 64 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.806452 | 0.005184 | 3.077138 | 0.25 | 0.100304 | 0.106495 | 0.117748 | 0.105603 | 0.261707 | 0.065015 | −0.01488 | 0.127545 | 0.138532 | 0.01504 | 0.021048 | 0.047601 | 65 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.645161 | 0.000103 | 1.378227 | 0.25 | −0.06134 | 0.090975 | −0.04119 | 0.089553 | −0.00342 | 0.138388 | −0.01058 | 0.101872 | −0.09817 | −0.13299 | −0.1736 | −0.18994 | 66 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.709677 | 0.000197 | 1.350626 | 0.25 | 0.112992 | 0.013352 | 0.072481 | −0.10939 | −0.00444 | −0.08442 | −0.00401 | 0.002746 | −0.16434 | −0.32656 | −0.12666 | −0.34394 | 67 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.451613 | 5.39E−05 | 1.397126 | 0.25 | 0.12118 | 0.132316 | 0.136844 | 0.10115 | 0.150545 | 0.258748 | −0.20306 | −0.17483 | −0.11676 | 0.02355 | −0.10854 | −0.19551 | 68 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.483871 | 8.97E−05 | 1.926084 | 0.25 | −0.09138 | −0.05504 | 0.017638 | 0.064249 | −0.04829 | 0.062232 | −0.19943 | −0.07825 | −0.08344 | −0.04651 | −0.22215 | −0.28417 | 69 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.193548 | 2.77E−05 | 2.386641 | 0.25 | −0.12828 | −0.15911 | −0.15047 | −0.14164 | 0.1153 | −0.01329 | −0.01234 | 0.612298 | 0.148664 | 0.109148 | −0.06294 | 0.091145 | 70 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.290323 | 8.45E−05 | 2.081109 | 0.25 | −0.24135 | −0.18003 | −0.2181 | −0.22709 | −0.02371 | −0.03411 | −0.02047 | 0.506327 | 0.041264 | −0.02675 | −0.2335 | −0.1761 | 71 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.580645 | 8.06E−05 | 1.603248 | 0.25 | 0.055441 | 0.227811 | 0.066799 | 0.317087 | 0.114815 | 0.298088 | −0.04087 | 0.107729 | 0.08227 | 0.003943 | 0.01055 | 0.007148 | 72 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.548387 | 8.76E−05 | 1.613159 | 0.25 | 0.073779 | 0.291659 | 0.139067 | 0.385265 | 0.102765 | 0.377637 | −0.29944 | −0.15777 | 0.037652 | −0.05384 | −0.08757 | −0.13289 | 73 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.483871 | 9.85E−05 | 1.938518 | 0.25 | −0.01626 | 0.04088 | 0.141096 | 0.110535 | 0.127334 | 0.175323 | −0.22795 | −0.14381 | 0.170464 | 0.233874 | −0.10699 | −0.16828 | 74 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.612903 | 0.000116 | 1.338248 | 0.125 | −0.00616 | 0.035339 | 0.081104 | 0.030919 | 0.003185 | −0.00483 | −0.12711 | −0.01018 | −0.07188 | −0.063 | −0.10928 | −0.23115 | 75 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.483871 | 7.76E−05 | 2.11918 | 0.125 | 0.030156 | 0.284813 | 0.175574 | 0.38868 | 0.150219 | 0.372955 | −0.0904 | −0.11759 | 0.084051 | 0.069521 | −0.08496 | −0.05311 | 76 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.258065 | 2.96E−05 | 2.034487 | 0.125 | −0.00091 | 0.022772 | 0.124536 | 0.203677 | 0.078578 | 0.117526 | −0.11443 | −0.15704 | 0.123057 | 0.018845 | −0.09775 | −0.01598 | 77 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.516129 | 5.55E−05 | 1.514663 | 0.125 | 0.11941 | 0.093835 | 0.077575 | 0.127642 | −0.01607 | 0.078739 | −0.14545 | −0.03137 | −0.08079 | −0.13546 | −0.14855 | −0.21465 | 78 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.129032 | 1.35E−05 | 2.646754 | 0.125 | 0.18118 | 0.05242 | 0.077405 | −0.09217 | 0.178192 | 0.014469 | −0.00936 | 0.106472 | 0.116696 | −0.06054 | −0.12376 | −0.15458 | 79 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.225806 | 3.55E−05 | 3.012591 | 0.125 | −0.02565 | −0.13236 | −0.00676 | −0.19552 | 0.129589 | −0.0761 | −0.17102 | 0.0299 | 0.167932 | 0.016627 | −0.03776 | −0.1181 | 80 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.580645 | 5.15E−05 | 1.170119 | 0.125 | 0.017532 | 0.084949 | 0.077258 | 0.133539 | 0.043746 | 0.127565 | −0.14755 | −0.13382 | −0.08657 | −0.14897 | −0.26989 | −0.27658 | 81 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.645161 | 0.000103 | 1.381591 | 0.125 | 0.248917 | 0.340116 | 0.241233 | 0.310043 | 0.206564 | 0.279072 | −0.10989 | 0.207032 | −0.09401 | −0.15034 | −0.16893 | −0.05832 | 82 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.548387 | 0.000133 | 1.345277 | 0.125 | −0.02342 | 0.004098 | −0.047 | −0.04376 | 0.110928 | 0.121342 | −0.16974 | −0.13971 | 0.069589 | −0.04623 | −0.27678 | −0.41519 | 83 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.258065 | 1.58E−05 | 2.202001 | 0.125 | −0.08607 | −0.15039 | −0.14605 | −0.20908 | −0.02048 | −0.07332 | −0.17449 | −0.12589 | 0.096499 | −0.03458 | −0.20708 | −0.26238 | 84 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.387097 | 8.78E−05 | 1.770855 | 0.125 | 0.034598 | 0.01581 | −0.13591 | −0.18141 | −0.00657 | −0.01675 | −0.03334 | 0.011886 | −0.23565 | −0.21221 | −0.34218 | −0.40995 | 85 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.387097 | 0.000123 | 2.029176 | 0.125 | −0.03592 | −0.08679 | 0.062623 | −0.10528 | 0.180879 | 0.006706 | −0.13189 | 0.161691 | 0.116513 | −0.08594 | −0.06572 | −0.14298 | 86 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.645161 | 0.000103 | 1.225558 | 0.125 | −0.07949 | −0.03669 | −0.08128 | −0.08693 | −0.02629 | 0.080201 | −0.14921 | 0.033648 | −0.20385 | −0.1167 | −0.38097 | −0.34935 | 87 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.612903 | 0.000104 | 1.888925 | 0.125 | 0.020622 | 0.070035 | 0.087113 | 0.102048 | 0.039499 | 0.066792 | −0.08518 | −0.01604 | −0.09401 | −0.11289 | −0.17322 | −0.13103 | 88 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.193548 | 1.74E−05 | 2.418695 | 0.125 | −0.13557 | −0.17476 | −0.11791 | −0.17686 | −0.03496 | −0.15912 | −0.37549 | −0.272 | 0.161375 | 0.018247 | −0.16996 | −0.26822 | 89 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.516129 | 7.58E−05 | 1.253948 | 0.125 | 0.036509 | 0.010628 | 0.141725 | 0.017241 | 0.122261 | 0.160282 | −0.06309 | 0.026205 | −0.05632 | 0.059551 | −0.23318 | −0.24523 | 90 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.16129 | 1.47E−05 | 2.456119 | 0.125 | −0.16295 | −0.20816 | −0.16676 | −0.18822 | 0.08135 | −0.00167 | −0.0786 | 0.576489 | −0.00463 | 0.115162 | −0.21855 | −0.01033 | 91 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.354839 | 7.63E−05 | 3.316778 | 0.125 | −0.11626 | −0.02604 | −0.10449 | −0.05353 | 0.016986 | 0.069936 | −0.30394 | 0.12299 | −0.1925 | −0.10214 | −0.28776 | −0.10214 | 92 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.193548 | 1.62E−05 | 2.412363 | 0.125 | −0.13734 | −0.19141 | −0.16381 | −0.2889 | −0.02339 | −0.16128 | −0.02124 | 0.274737 | −0.04729 | −0.18587 | 0.012935 | −0.19949 | 93 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.322581 | 3.47E−05 | 1.949339 | 0.125 | −0.10342 | −0.11971 | 0.019197 | −0.13541 | −0.04187 | −0.10828 | −0.17107 | 0.146771 | 0.036278 | −0.14663 | −0.01967 | −0.16541 | 94 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.290323 | 3.04E−05 | 1.82547 | 0.125 | −0.15119 | −0.17061 | −0.08287 | −0.12982 | −0.243 | −0.25182 | −0.03586 | −0.12437 | 0.070253 | −0.02258 | −0.03045 | −0.16083 | 95 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.645161 | 8.90E−05 | 1.068164 | 0.125 | 0.300086 | 0.36618 | 0.214005 | 0.374607 | 0.217784 | 0.362855 | −0.21423 | −0.22355 | −0.04784 | −0.01631 | −0.12889 | −0.09994 | 96 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.451613 | 6.71E−05 | 1.668141 | 0.125 | 0.083596 | 0.08778 | 0.321794 | 0.280365 | 0.230901 | 0.150406 | −0.08168 | 0.135616 | 0.081573 | −0.03048 | 0.088111 | 0.142922 | 97 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.483871 | 7.65E−05 | 1.865476 | 0.125 | −0.03689 | 0.31638 | 0.012716 | 0.17913 | 0.039199 | 0.233694 | −0.2443 | −0.05303 | −0.31027 | −0.22509 | −0.46209 | −0.11746 | 98 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.064516 | 1.34E−05 | 4.600896 | 0.125 | 0.021812 | 0.059364 | −0.0152 | −0.05313 | 0.261304 | 0.112994 | −0.10308 | 0.027125 | 0.21792 | 0.288728 | 0.013887 | 0.056787 | 99 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.419355 | 8.31E−05 | 2.570026 | 0.125 | 0.068702 | 0.195934 | 0.043168 | 0.126618 | 0.088037 | 0.223124 | −0.34319 | −0.07332 | −0.22531 | −0.21582 | −0.37915 | −0.35437 | 100 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| Bacteroides | | | | | | | | | | | | | | | | | |
| Bacteria;Bacteroidetes; | 0.387097 | 0.000329 | 2.267031 | 0.5 | −0.33905 | −0.32529 | −0.34922 | −0.33439 | −0.05574 | −0.09823 | −0.03999 | 0.329543 | −0.13576 | −0.13193 | −0.34601 | −0.25653 | 101 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| unclassified | | | | | | | | | | | | | | | | | |
| Bacteria;Bacteroidetes; | 0.258065 | 0.000247 | 3.094617 | 0.5 | −0.12125 | −0.11641 | −0.14199 | −0.09965 | 0.186037 | 0.053632 | −0.07799 | 0.327853 | 0.079231 | 0.130249 | −0.17421 | 0.095288 | 102 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| unclassified | | | | | | | | | | | | | | | | | |
| Bacteria;Bacteroidetes; | 0.064516 | 7.95E−06 | 4.13495 | 0.125 | 0.005547 | 0.047283 | −0.00196 | 0.012227 | 0.208603 | 0.137159 | −0.31984 | −0.1684 | 0.110202 | 0.1361 | −0.09643 | 0.003693 | 103 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| unclassified | | | | | | | | | | | | | | | | | |
| Bacteria;Bacteroidetes; | 0.354839 | 4.87E−05 | 2.077535 | 0.125 | 0.027502 | −0.00528 | 0.098641 | −0.00071 | 0.170685 | 0.075177 | −0.13812 | −0.08793 | 0.031553 | −0.17117 | −0.1332 | −0.08431 | 104 |
| Bacteroidia; | | | | | | | | | | | | | | | | | |
| Bacteroidales; | | | | | | | | | | | | | | | | | |
| Bacteroidaceae; | | | | | | | | | | | | | | | | | |
| unclassified | | | | | | | | | | | | | | | | | |
| Bacteria;Bacteroidetes; | 0.16129 | 5.85E−05 | 4.475447 | 0.75 | −0.22233 | −0.23794 | −0.29024 | −0.32787 | −0.0769 | −0.07994 | −0.22245 | −0.01558 | −0.10932 | 0.009289 | −0.29781 | −0.27075 | 105 |
| Bacteroidia; | | | | | | | | | | | | | | | | | |
| Bacteroidales; | | | | | | | | | | | | | | | | | |
| Porphyromonadaceae; | | | | | | | | | | | | | | | | | |
| Barnesiella | | | | | | | | | | | | | | | | | |
| Bacteria;Bacteroidetes; | 0.032258 | 3.61E−06 | 5.567764 | 0.125 | 0.251089 | 0.279281 | 0.15247 | 0.149412 | 0.247628 | 0.187992 | −0.02415 | −0.09327 | −0.05281 | −0.05683 | 0.016491 | 0.002099 | 106 |
| Bacteroidia; | | | | | | | | | | | | | | | | | |
| Bacteroidales; | | | | | | | | | | | | | | | | | |
| Porphyromonadaceae; | | | | | | | | | | | | | | | | | |
| Barnesiella | | | | | | | | | | | | | | | | | |
| Bacteria;Bacteroidetes; | 0.290323 | 0.000177 | 2.948035 | 0.375 | −0.24599 | −0.18571 | −0.36516 | −0.25176 | −0.14929 | −0.16154 | −0.05854 | 0.301391 | −0.09683 | −0.12383 | −0.27257 | −0.15989 | 107 |
| Bacteroidia; | | | | | | | | | | | | | | | | | |
| Bacteroidales; | | | | | | | | | | | | | | | | | |
| Porphyromonadaceae; | | | | | | | | | | | | | | | | | |
| Coprobacter | | | | | | | | | | | | | | | | | |
| Bacteria;Bacteroidetes; | 0.774194 | 0.002326 | 1.199726 | 0.75 | −0.09765 | −0.0383 | −0.19552 | −0.12389 | 0.027925 | 0.035425 | −0.1011 | 0.14689 | 0.10142 | 0.154892 | −0.11669 | −0.00165 | 108 |
| Bacteroidia; | | | | | | | | | | | | | | | | | |
| Bacteroidales; | | | | | | | | | | | | | | | | | |
| Porphyromonadaceae; | | | | | | | | | | | | | | | | | |
| Odoribacter | | | | | | | | | | | | | | | | | |
| Bacteria;Bacteroidetes; | 0.387097 | 0.001449 | 2.381053 | 0.75 | −0.3395 | −0.29853 | −0.26702 | −0.27118 | −0.06137 | −0.1063 | −0.1848 | 0.319923 | 0.067847 | 0.072863 | −0.20112 | −0.22379 | 109 |
| Bacteroidia; | | | | | | | | | | | | | | | | | |
| Bacteroidales; | | | | | | | | | | | | | | | | | |
| Porphyromonadaceae; | | | | | | | | | | | | | | | | | |
| Parabacteroides | | | | | | | | | | | | | | | | | |
| Bacteria;Bacteroidetes; | 0.322581 | 0.000246 | 2.971111 | 0.625 | −0.25699 | −0.29297 | −0.17402 | −0.26271 | 0.088522 | −0.08518 | −0.18043 | 0.076597 | 0.073959 | 0.015091 | −0.23637 | −0.33215 | 110 |
| Bacteroidia; | | | | | | | | | | | | | | | | | |
| Bacteroidales; | | | | | | | | | | | | | | | | | |
| Porphyromonadaceae; | | | | | | | | | | | | | | | | | |
| Parabacteroides | | | | | | | | | | | | | | | | | |
| Bacteria;Bacteroidetes; | 0.83871 | 0.009247 | 1.036926 | 0.625 | −0.01924 | −0.00052 | −0.01332 | 0.015953 | 0.178604 | 0.16988 | −0.29975 | −0.13514 | 0.025398 | 0.036181 | −0.26164 | −0.08131 | 111 |
| Bacteroidia; | | | | | | | | | | | | | | | | | |
| Bacteroidales; | | | | | | | | | | | | | | | | | |
| Porphyromonadaceae; | | | | | | | | | | | | | | | | | |
| Parabacteroides | | | | | | | | | | | | | | | | | |
| Bacteria;Bacteroidetes; | 0.419355 | 0.000154 | 1.61906 | 0.375 | −0.06207 | 0.02614 | −0.0141 | 0.014746 | 0.158927 | 0.174473 | −0.12439 | 0.227845 | −0.03741 | −0.10039 | −0.1693 | −0.06613 | 112 |
| Bacteroidia; | | | | | | | | | | | | | | | | | |
| Bacteroidales; | | | | | | | | | | | | | | | | | |
| Porphyromonadaceae; | | | | | | | | | | | | | | | | | |
| Parabacteroides | | | | | | | | | | | | | | | | | |
| Bacteria;Bacteroidetes; | 0.129032 | 2.22E−05 | 3.077303 | 0.25 | −0.11869 | −0.18498 | −0.10908 | −0.12625 | −0.00884 | −0.02665 | −0.17594 | 0.057815 | −0.06268 | −0.10843 | −0.21571 | −0.05231 | 113 |
| Bacteroidia; | | | | | | | | | | | | | | | | | |
| Bacteroidales; | | | | | | | | | | | | | | | | | |
| Porphyromonadaceae; | | | | | | | | | | | | | | | | | |
| unclassified | | | | | | | | | | | | | | | | | |
| Bacteria;Bacteroidetes; | 0.064516 | 0.000229 | 4.501627 | 0.875 | −0.31043 | −0.35592 | −0.33936 | −0.46016 | −0.15693 | −0.26254 | −0.26647 | 0.081546 | −0.12287 | −0.20656 | −0.34852 | −0.48421 | 114 |
| Bacteroidia; | | | | | | | | | | | | | | | | | |
| Bacteroidales; | | | | | | | | | | | | | | | | | |
| Prevotellaceae; | | | | | | | | | | | | | | | | | |
| Paraprevotella | | | | | | | | | | | | | | | | | |
| Bacteria;Bacteroidetes; | 0.129032 | 0.000325 | 5.447453 | 0.875 | −0.35292 | −0.34576 | −0.39011 | −0.42804 | −0.17288 | −0.20909 | −0.29235 | 0.101877 | −0.12164 | −0.13783 | −0.3936 | −0.42932 | 115 |
| Bacteroidia; | | | | | | | | | | | | | | | | | |
| Bacteroidales; | | | | | | | | | | | | | | | | | |
| Prevotellaceae; | | | | | | | | | | | | | | | | | |
| Paraprevotella | | | | | | | | | | | | | | | | | |
| Bacteria;Bacteroidetes; | 0.032258 | 1.08E−05 | 5.567764 | 0.625 | −0.25573 | −0.25775 | −0.19383 | −0.27177 | 0.078335 | −0.05954 | −0.30769 | 0.281946 | 0.040183 | −0.05117 | −0.22198 | −0.18139 | 116 |
| Bacteroidia; | | | | | | | | | | | | | | | | | |
| Bacteroidales; | | | | | | | | | | | | | | | | | |
| Prevotellaceae; | | | | | | | | | | | | | | | | | |
| Paraprevotella | | | | | | | | | | | | | | | | | |
| Bacteria;Bacteroidetes; | 0.032258 | 7.23E−06 | 5.567764 | 0.375 | −0.27707 | −0.1761 | −0.30428 | −0.26273 | −0.08605 | −0.10433 | −0.16267 | 0.536807 | −0.11285 | −0.09885 | −0.27995 | −0.18322 | 117 |
| Bacteroidia; | | | | | | | | | | | | | | | | | |
| Bacteroidales; | | | | | | | | | | | | | | | | | |
| Prevotellaceae; | | | | | | | | | | | | | | | | | |
| Paraprevotella | | | | | | | | | | | | | | | | | |
| Bacteria;Bacteroidetes; | 0.032258 | 9.03E−06 | 5.567764 | 0.375 | −0.29075 | −0.24732 | −0.30218 | −0.29352 | −0.05955 | −0.11248 | −0.21845 | 0.15172 | −0.03288 | −0.03761 | −0.22662 | −0.23576 | 118 |
| Bacteroidia; | | | | | | | | | | | | | | | | | |
| Bacteroidales; | | | | | | | | | | | | | | | | | |
| Prevotellaceae; | | | | | | | | | | | | | | | | | |
| Paraprevotella | | | | | | | | | | | | | | | | | |
| Bacteria;Bacteroidetes; | 0 | 0 | | 0.125 | −0.33414 | −0.26701 | −0.42582 | −0.36199 | −0.16333 | −0.19262 | −0.25347 | −0.03432 | −0.18916 | −0.2404 | −0.43217 | −0.39094 | 119 |
| Bacteroidia; | | | | | | | | | | | | | | | | | |
| Bacteroidales; | | | | | | | | | | | | | | | | | |
| Prevotellaceae; | | | | | | | | | | | | | | | | | |
| Paraprevotella | | | | | | | | | | | | | | | | | |
| Bacteria;Bacteroidetes; | 0.032258 | 3.61E−06 | 5.567764 | 0.125 | 0.100899 | 0.111514 | 0.042236 | 0.047102 | 0.126417 | 0.094156 | −0.16898 | −0.08805 | 0.159093 | 0.113724 | −0.07997 | −0.0348 | 120 |
| Bacteroidia; | | | | | | | | | | | | | | | | | |
| Bacteroidales; | | | | | | | | | | | | | | | | | |
| Prevotellaceae; | | | | | | | | | | | | | | | | | |
| Paraprevotella | | | | | | | | | | | | | | | | | |
| Bacteria;Bacteroidetes; | 0.032258 | 3.61E−06 | 5.567764 | 0.125 | −0.1731 | −0.15778 | −0.12204 | −0.18236 | 0.031212 | −0.03628 | −0.39355 | −0.2144 | 0.042538 | 0.117591 | −0.16786 | −0.19392 | 121 |
| Bacteroidia; | | | | | | | | | | | | | | | | | |
| Bacteroidales; | | | | | | | | | | | | | | | | | |
| Prevotellaceae; | | | | | | | | | | | | | | | | | |
| Paraprevotella | | | | | | | | | | | | | | | | | |
| Bacteria;Bacteroidetes; | 0.032258 | 3.61E−06 | 5.567764 | 0.125 | −0.10152 | −0.19548 | −0.11404 | −0.25544 | −0.01499 | −0.18205 | −0.00125 | 0.341302 | 0.117439 | −0.11443 | −0.03146 | −0.19576 | 122 |
| Bacteroidia; | | | | | | | | | | | | | | | | | |
| Bacteroidales; | | | | | | | | | | | | | | | | | |
| Prevotellaceae; | | | | | | | | | | | | | | | | | |
| Paraprevotella | | | | | | | | | | | | | | | | | |
| Bacteria;Bacteroidetes; | 0.032258 | 1.81E−06 | 5.567764 | 0.125 | −0.34306 | −0.33662 | −0.39783 | −0.42431 | −0.18807 | −0.23831 | −0.28708 | 0.008713 | −0.09043 | −0.12406 | −0.35311 | −0.47539 | 123 |
| Bacteroidia; | | | | | | | | | | | | | | | | | |
| Bacteroidales; | | | | | | | | | | | | | | | | | |
| Prevotellaceae; | | | | | | | | | | | | | | | | | |
| Paraprevotella | | | | | | | | | | | | | | | | | |
| Bacteria;Bacteroidetes; | 0.709677 | 0.001257 | 1.349633 | 0.875 | −0.14779 | −0.13059 | −0.22517 | −0.22966 | −0.04285 | −0.08739 | −0.2886 | −0.14735 | 0.117229 | 0.082537 | −0.25908 | −0.4149 | 124 |
| Bacteroidia; | | | | | | | | | | | | | | | | | |
| Bacteroidales; | | | | | | | | | | | | | | | | | |
| Rikenellaceae;Alistipes | | | | | | | | | | | | | | | | | |
| Bacteria;Bacteroidetes; | 0.741935 | 0.005672 | 1.017888 | 0.875 | −0.29982 | −0.25821 | −0.39393 | −0.30233 | −0.27175 | −0.23423 | −0.0002 | 0.564093 | 0.045529 | 0.073935 | −0.1588 | −0.00704 | 125 |
| Bacteroidia; | | | | | | | | | | | | | | | | | |
| Bacteroidales; | | | | | | | | | | | | | | | | | |
| Rikenellaceae;Alistipes | | | | | | | | | | | | | | | | | |
| Bacteria;Bacteroidetes; | 0.483871 | 0.000666 | 1.913532 | 0.75 | −0.21537 | −0.21542 | −0.29222 | −0.22629 | −0.10854 | −0.13841 | −0.05443 | −0.07755 | 0.037987 | −0.0145 | −0.20592 | −0.3205 | 126 |
| Bacteroidia; | | | | | | | | | | | | | | | | | |
| Bacteroidales; | | | | | | | | | | | | | | | | | |
| Rikenellaceae;Alistipes | | | | | | | | | | | | | | | | | |
| Bacteria;Bacteroidetes; | 0.741935 | 0.004045 | 2.298836 | 0.125 | −0.06961 | −0.13905 | −0.05839 | −0.05725 | 0.184976 | −0.05228 | −0.05381 | −0.00932 | 0.351176 | 0.040554 | 0.0807 | 0.130744 | 127 |
| Bacteroidia; | | | | | | | | | | | | | | | | | |
| Bacteroidales; | | | | | | | | | | | | | | | | | |
| Rikenellaceae;Alistipes | | | | | | | | | | | | | | | | | |
| Bacteria;Bacteroidetes; | 0.322581 | 0.000123 | 1.956982 | 0.125 | −0.13202 | −0.02982 | −0.23947 | −0.13821 | −0.16748 | −0.0771 | −0.18861 | −0.18002 | −0.08982 | 0.009799 | −0.23587 | −0.19313 | 128 |
| Bacteroidia; | | | | | | | | | | | | | | | | | |
| Bacteroidales; | | | | | | | | | | | | | | | | | |
| Rikenellaceae;unclassified | | | | | | | | | | | | | | | | | |
| Bacteria;Bacteroidetes; | 0.193548 | 7.64E−05 | 2.684514 | 0.125 | −0.27331 | −0.11604 | −0.40617 | −0.27301 | −0.27544 | −0.08844 | −0.3757 | −0.20697 | −0.1623 | 0.044043 | −0.40574 | −0.44811 | 129 |
| Bacteroidia; | | | | | | | | | | | | | | | | | |
| Bacteroidales; | | | | | | | | | | | | | | | | | |
| Rikenellaceae;unclassified | | | | | | | | | | | | | | | | | |
| Bacteria;Bacteroidetes; | 0.032258 | 9.03E−06 | 5.567764 | 0.375 | −0.25131 | −0.22736 | −0.20157 | −0.27257 | 0.101311 | 0.013168 | −0.2862 | 0.183936 | 0.012623 | −0.03975 | −0.30331 | −0.2839 | 130 |
| Bacteroidia; | | | | | | | | | | | | | | | | | |
| Bacteroidales; | | | | | | | | | | | | | | | | | |
| unclassified;unclassified | | | | | | | | | | | | | | | | | |
| Bacteria;Bacteroidetes; | 0.064516 | 2.42E−05 | 4.780344 | 0.25 | −0.24334 | −0.28681 | −0.20357 | −0.33157 | 0.077859 | −0.099 | −0.27575 | 0.184275 | 0.019624 | −0.04636 | −0.29168 | −0.29865 | 131 |
| Bacteroidia; | | | | | | | | | | | | | | | | | |
| Bacteroidales; | | | | | | | | | | | | | | | | | |
| unclassified;unclassified | | | | | | | | | | | | | | | | | |
| Bacteria;Bacteroidetes; | 0.258065 | 7.30E−05 | 2.042281 | 0.25 | −0.15787 | −0.14742 | −0.06513 | −0.07133 | 0.084628 | 0.065756 | −0.03722 | 0.194294 | 0.21677 | −0.00692 | −0.12881 | 0.001039 | 132 |
| Bacteroidia; | | | | | | | | | | | | | | | | | |
| Bacteroidales; | | | | | | | | | | | | | | | | | |
| unclassified;unclassified | | | | | | | | | | | | | | | | | |
| Bacteria;Bacteroidetes; | 0.032258 | 1.08E−05 | 5.567764 | 0.75 | −0.3043 | −0.39606 | −0.38814 | −0.34503 | −0.15336 | −0.17582 | −0.20194 | −0.15547 | −0.0685 | −0.01877 | −0.36761 | −0.20521 | 133 |
| Bacteroidia; | | | | | | | | | | | | | | | | | |
| Bacteroidales; | | | | | | | | | | | | | | | | | |
| unclassified;unclassified | | | | | | | | | | | | | | | | | |
| Bacteria;Bacteroidetes; | 0.032258 | 5.42E−06 | 5.567764 | 0.625 | −0.21599 | −0.31891 | −0.21856 | −0.26249 | 0.009303 | −0.1255 | −0.29833 | −0.17745 | 0.094686 | −0.03566 | −0.24489 | −0.17918 | 134 |
| Bacteroidia; | | | | | | | | | | | | | | | | | |
| Bacteroidales; | | | | | | | | | | | | | | | | | |
| unclassified;unclassified | | | | | | | | | | | | | | | | | |
| Bacteria;Firmicutes; | 0.032258 | 0.000398 | 5.567764 | 0.125 | 0.259448 | 0.396227 | 0.259487 | 0.291423 | 0.259553 | 0.420272 | −0.04351 | −0.02392 | −0.08746 | −0.05469 | −0.15989 | −0.10828 | 135 |
| Bacilli;Lactobacillales; | | | | | | | | | | | | | | | | | |
| Lactobacillaceae; | | | | | | | | | | | | | | | | | |
| Lactobacillus | | | | | | | | | | | | | | | | | |
| Bacteria;Firmicutes; | 1 | 0.004274 | 1.104826 | 0.125 | 0.469636 | 0.426206 | 0.407451 | 0.458781 | 0.20646 | 0.31916 | −0.03279 | −0.08774 | 0.033779 | −0.0788 | 0.062569 | 0.095465 | 136 |
| Bacilli;Lactobacillales; | | | | | | | | | | | | | | | | | |
| Streptococcaceae; | | | | | | | | | | | | | | | | | |
| Streptococcus | | | | | | | | | | | | | | | | | |
| Bacteria;Firmicutes; | 0.580645 | 0.001247 | 1.575506 | 0.125 | 0.013688 | 0.218767 | 0.099229 | 0.239814 | 0.068773 | 0.164239 | −0.07437 | −0.07131 | −0.18868 | −0.10319 | −0.16662 | −0.00726 | 137 |
| Bacilli;unclassified; | | | | | | | | | | | | | | | | | |
| unclassified;unclassified | | | | | | | | | | | | | | | | | |
| Bacteria;Firmicutes; | 0.193548 | 1.59E−05 | 2.685929 | 0.125 | −0.09811 | 0.011977 | 0.064984 | 0.073181 | −0.0782 | −0.04375 | −0.29699 | −0.11842 | 0.08007 | 0.019032 | −0.14956 | −0.02553 | 138 |
| Bacilli;unclassified; | | | | | | | | | | | | | | | | | |
| unclassified;unclassified | | | | | | | | | | | | | | | | | |
| Bacteria;Firmicutes; | 0.064516 | 1.08E−05 | 4.101578 | 0.75 | 0.143946 | 0.135041 | 0.06959 | 0.032267 | 0.135024 | 0.120691 | −0.10203 | −0.15019 | −0.15262 | −0.05002 | −0.18702 | −0.04846 | 139 |
| Clostridia;Clostridiales; | | | | | | | | | | | | | | | | | |
| Eubacteriaceae; | | | | | | | | | | | | | | | | | |
| unclassified | | | | | | | | | | | | | | | | | |
| Bacteria;Firmicutes; | 0.903226 | 0.000663 | 1.29878 | 0.25 | −0.29081 | −0.23641 | −0.0216 | −0.01842 | 0.052878 | −0.04143 | −0.07742 | −0.04289 | −0.09087 | 0.066245 | −0.23551 | −0.00969 | 140 |
| Clostridia;Clostridiales; | | | | | | | | | | | | | | | | | |
| Lachnospiraceae; | | | | | | | | | | | | | | | | | |
| Blautia | | | | | | | | | | | | | | | | | |
| Bacteria;Firmicutes; | 0.935484 | 0.015708 | 0.740071 | 0.25 | 0.287768 | 0.302013 | 0.322231 | 0.266703 | 0.243127 | 0.307685 | −0.19289 | −0.21926 | −0.06777 | −0.02709 | −0.14178 | −0.00232 | 141 |
| Clostridia;Clostridiales; | | | | | | | | | | | | | | | | | |
| Lachnospiraceae; | | | | | | | | | | | | | | | | | |
| Blautia | | | | | | | | | | | | | | | | | |
| Bacteria;Firmicutes; | 0.290323 | 6.30E−05 | 2.855144 | 0.125 | 0.077568 | 0.040852 | −0.05737 | −0.10486 | 0.027395 | −0.00857 | −0.14237 | −0.10358 | −0.00952 | −0.06168 | −0.07561 | −0.20758 | 142 |
| Clostridia;Clostridiales; | | | | | | | | | | | | | | | | | |
| Lachnospiraceae; | | | | | | | | | | | | | | | | | |
| Blautia | | | | | | | | | | | | | | | | | |
| Bacteria;Firmicutes; | 0.096774 | 1.78E−05 | 4.559169 | 0.125 | −0.02668 | −0.21749 | −0.13829 | −0.24898 | −0.01564 | −0.11864 | −0.04214 | −0.04983 | −0.14505 | 0.012671 | −0.21957 | −0.2342 | 143 |
| Clostridia;Clostridiales; | | | | | | | | | | | | | | | | | |
| Lachnospiraceae;Blautia | | | | | | | | | | | | | | | | | |
| Bacteria;Firmicutes; | 0.806452 | 0.001298 | 0.827767 | 0.5 | 0.148745 | 0.104663 | 0.022766 | −0.07688 | 0.10884 | 0.058388 | −0.23841 | −0.26915 | −0.09844 | 0.014963 | −0.33605 | −0.31944 | 144 |
| Clostridia;Clostridiales; | | | | | | | | | | | | | | | | | |
| Lachnospiraceae; | | | | | | | | | | | | | | | | | |
| Coprococcus | | | | | | | | | | | | | | | | | |
| Bacteria;Firmicutes; | 0.83871 | 0.004297 | 0.745378 | 0.25 | 0.064777 | 0.284704 | 0.0576 | 0.193989 | 0.152086 | 0.337456 | −0.00367 | 0.050339 | −0.16214 | −0.10749 | −0.31621 | −0.21897 | 145 |
| Clostridia;Clostridiales; | | | | | | | | | | | | | | | | | |
| Lachnospiraceae; | | | | | | | | | | | | | | | | | |
| Coprococcus | | | | | | | | | | | | | | | | | |
| Bacteria;Firmicutes; | 0.290323 | 2.46E−05 | 1.917127 | 0.125 | 0.216781 | −0.0174 | 0.164269 | −0.09122 | 0.303701 | 0.045757 | −0.12319 | −0.06116 | −0.0571 | 0.001714 | −0.2469 | −0.17564 | 146 |
| Clostridia;Clostridiales; | | | | | | | | | | | | | | | | | |
| Lachnospiraceae; | | | | | | | | | | | | | | | | | |
| Coprococcus | | | | | | | | | | | | | | | | | |
| Bacteria;Firmicutes; | 1 | 0.012882 | 0.647079 | 0.125 | −0.33311 | −0.31826 | −0.19174 | −0.23886 | −0.22014 | −0.29375 | −0.07027 | 0.024418 | 0.026001 | 0.036058 | −0.13086 | −0.06177 | 147 |
| Clostridia;Clostridiales; | | | | | | | | | | | | | | | | | |
| Lachnospiraceae; | | | | | | | | | | | | | | | | | |
| Fusicatenibacter | | | | | | | | | | | | | | | | | |
| Bacteria;Firmicutes; | 0.870968 | 0.001017 | 1.291857 | 0.125 | −0.29498 | −0.1589 | −0.20621 | −0.15566 | −0.18125 | −0.23637 | −0.01017 | −0.0629 | 0.272724 | 0.19773 | 0.023366 | −0.0662 | 148 |
| Clostridia;Clostridiales; | | | | | | | | | | | | | | | | | |
| Lachnospiraceae;_ | | | | | | | | | | | | | | | | | |
| Lachnospiracea_ | | | | | | | | | | | | | | | | | |
| incertae_sedis | | | | | | | | | | | | | | | | | |
| Bacteria;Firmicutes; | 0.935484 | 0.006349 | 0.795346 | 0.125 | −0.00729 | 0.246206 | −0.01296 | 0.320028 | −0.04313 | 0.254515 | −0.01955 | −0.09421 | −0.04279 | −0.02786 | −0.21792 | −0.14704 | 149 |
| Clostridia;Clostridiales; | | | | | | | | | | | | | | | | | |
| Lachnospiraceae; | | | | | | | | | | | | | | | | | |
| Lachnospiracea_incertae_ | | | | | | | | | | | | | | | | | |
| sedis | | | | | | | | | | | | | | | | | |
| Bacteria;Firmicutes; | 0.83871 | 0.003171 | 1.429324 | 0.25 | 0.248416 | 0.340629 | 0.196988 | 0.287071 | 0.314399 | 0.277165 | −0.05937 | 0.084751 | 0.067165 | −0.02834 | −0.0113 | 0.134147 | 150 |
| Clostridia;Clostridiales; | | | | | | | | | | | | | | | | | |
| Lachnospiraceae; | | | | | | | | | | | | | | | | | |
| Roseburia | | | | | | | | | | | | | | | | | |
| Bacteria;Firmicutes; | 0.483871 | 0.000115 | 1.613401 | 0.125 | 0.073513 | 0.154021 | 0.060395 | 0.163511 | 0.163459 | 0.138618 | −0.23976 | −0.09928 | −0.09706 | −0.06248 | −0.13604 | 0.085369 | 151 |
| Clostridia;Clostridiales; | | | | | | | | | | | | | | | | | |
| Lachnospiraceae; | | | | | | | | | | | | | | | | | |
| Roseburia | | | | | | | | | | | | | | | | | |
| Bacteria;Firmicutes; | 0.870968 | 0.001441 | 1.444082 | 0.125 | 0.162584 | 0.122183 | 0.248874 | 0.182657 | 0.117075 | 0.042934 | −0.06906 | 0.015344 | −0.00491 | 0.018526 | 0.006228 | 0.09007 | 152 |
| Clostridia;Clostridiales; | | | | | | | | | | | | | | | | | |
| Lachnospiraceae; | | | | | | | | | | | | | | | | | |
| Roseburia | | | | | | | | | | | | | | | | | |
| Bacteria;Firmicutes; | 0.806452 | 0.001178 | 1.147206 | 0.375 | −0.19696 | −0.18676 | 0.0608 | −0.01691 | 0.14555 | 0.005382 | −0.14455 | −0.08938 | −0.05041 | 0.097777 | −0.22744 | −0.06374 | 153 |
| Clostridia;Clostridiales; | | | | | | | | | | | | | | | | | |
| Lachnospiraceae; | | | | | | | | | | | | | | | | | |
| unclassified; | | | | | | | | | | | | | | | | | |
| Bacteria;Firmicutes; | 0.83871 | 0.000832 | 1.156287 | 0.375 | 0.108255 | 0.18593 | 0.093987 | 0.077867 | 0.144957 | 0.188025 | −0.16752 | −0.14882 | 0.137956 | 0.197466 | 0.003075 | −0.0053 | 154 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.419355 | 9.65E−05 | 1.486352 | 0.25 | 0.194041 | 0.170046 | 0.081818 | 0.082144 | 0.165658 | 0.07241 | −0.02349 | −0.03136 | −0.07251 | −0.12503 | −0.07619 | −0.03608 | 155 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.387097 | 0.000247 | 2.359666 | 0.25 | 0.5643 | 0.212354 | 0.552316 | 0.215954 | 0.547677 | 0.196383 | −0.27313 | −0.16948 | 0.139608 | −0.1315 | 0.107724 | 0.062189 | 156 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.548387 | 6.30E−05 | 1.392664 | 0.25 | 0.073317 | 0.03048 | 0.099449 | −0.03365 | −0.11458 | −0.14466 | −0.08073 | −0.20785 | −0.26264 | −0.2478 | −0.19674 | −0.24583 | 157 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.419355 | 5.13E−05 | 1.551209 | 0.25 | 0.1615 | −0.03975 | 0.148633 | −0.08462 | −0.09246 | −0.19152 | −0.22101 | −0.23017 | −0.19363 | −0.23976 | −0.08721 | −0.24907 | 158 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.419355 | 3.68E−05 | 1.539135 | 0.25 | 0.038778 | −0.03063 | −0.01565 | −0.16011 | 0.038242 | −0.03067 | −0.25404 | −0.10422 | −0.04523 | −0.14024 | −0.21197 | −0.30399 | 159 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.806452 | 0.00094 | 1.338159 | 0.25 | −0.21196 | −0.22108 | 0.022293 | −0.07102 | 0.059295 | −0.00131 | −0.18043 | −0.10468 | −0.14488 | 0.038069 | −0.20731 | −0.04943 | 160 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.709677 | 0.000141 | 0.912702 | 0.125 | −0.04391 | −0.10808 | −0.05764 | −0.21829 | −0.00707 | −0.10083 | −0.00818 | −0.13058 | −0.14525 | −0.1906 | −0.28461 | −0.38194 | 161 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.290323 | 1.78E−05 | 1.851082 | 0.125 | −0.03073 | −0.17945 | −0.09411 | −0.28665 | −0.16225 | −0.26737 | −0.12238 | −0.0774 | −0.094 | −0.28327 | −0.16246 | −0.3282 | 162 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.354839 | 3.02E−05 | 1.566251 | 0.125 | −0.01453 | −0.02882 | −0.01578 | −0.08179 | 0.071879 | 0.152385 | −0.17793 | −0.16372 | −0.11869 | 0.025625 | −0.28857 | −0.2476 | 163 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.580645 | 0.000147 | 1.350701 | 0.125 | −0.14391 | −0.02344 | 0.024915 | 0.048403 | −0.02838 | −0.04997 | −0.34741 | −0.16324 | −0.16251 | −0.08332 | −0.31865 | −0.17934 | 164 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.290323 | 3.12E−05 | 1.999477 | 0.125 | 0.125929 | −0.02234 | 0.106254 | −0.06672 | 0.113241 | −0.02706 | −0.22185 | −0.19869 | −0.01836 | −0.11282 | −0.08799 | −0.20375 | 165 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.129032 | 9.75E−06 | 2.922283 | 0.125 | −0.23809 | −0.23851 | −0.1839 | −0.2895 | −0.21853 | −0.24267 | −0.04185 | −0.00954 | −0.04671 | −0.22481 | −0.12439 | −0.31806 | 166 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.354839 | 2.86E−05 | 1.555659 | 0.125 | 0.000347 | −0.14538 | 0.079571 | −0.16609 | 0.174327 | 0.013143 | −0.31914 | −0.20643 | 0.07713 | 0.027557 | −0.17824 | −0.27831 | 167 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.419355 | 5.66E−05 | 1.588083 | 0.125 | 0.166283 | 0.202112 | 0.257263 | 0.201822 | 0.233977 | 0.180435 | −0.09185 | −0.13024 | 0.054926 | 0.15436 | −0.03618 | 0.152277 | 168 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.516129 | 6.42E−05 | 1.303103 | 0.125 | −0.02687 | −0.06156 | 0.068447 | −0.08636 | −0.07908 | 0.10512 | −0.26152 | −0.06474 | −0.20488 | −0.20088 | −0.22916 | −0.2827 | 169 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.290323 | 2.01E−05 | 1.90503 | 0.125 | −0.21153 | −0.17817 | −0.11659 | −0.2666 | −0.20251 | 0.2182 | −0.02813 | 0.024564 | −0.05895 | −0.21571 | −0.05373 | −0.30333 | 170 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.516129 | 6.64E−05 | 1.286891 | 0.125 | 0.100103 | 0.020285 | 0.07548 | −0.0793 | 0.151109 | 0.050176 | −0.13972 | −0.09858 | −0.13604 | −0.03988 | −0.13954 | −0.16098 | 171 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.516129 | 5.70E−05 | 1.376118 | 0.125 | −0.1594 | −0.17373 | −0.0635 | −0.2273 | −0.00152 | −0.08264 | −0.13716 | −0.12677 | 0.004291 | −0.13261 | −0.15526 | −0.31017 | 172 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.677419 | 7.77E−05 | 1.0313 | 0.125 | −0.25344 | −0.30665 | −0.033147 | −0.37974 | −0.22815 | −0.26329 | −0.10649 | −0.09691 | −0.23665 | −0.22181 | −0.3232 | −0.35662 | 173 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.516129 | 7.23E−05 | 2.745631 | 0.125 | −0.12665 | −0.21483 | −0.11856 | −0.22261 | −0.00577 | −0.10354 | −0.17646 | −0.06545 | −0.03224 | 0.04714 | −0.02167 | −0.17059 | 174 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.032258 | 1.08E−05 | 5.567764 | 0.125 | −0.45873 | −0.29892 | −0.30063 | −0.24257 | −0.12632 | −0.1247 | −0.06069 | 0.318177 | −0.02745 | −0.15738 | −0.17476 | −0.08702 | 175 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.225806 | 2.07E−05 | 2.268131 | 0.125 | 0.201702 | 0.189911 | 0.173137 | 0.121129 | 0.143614 | 0.073824 | −0.05862 | −0.10151 | −0.06856 | −0.03868 | −0.00596 | 0.070802 | 176 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.516129 | 3.83E−05 | 1.564341 | 0.125 | −0.22059 | −0.20666 | −0.10442 | −0.23489 | −0.21328 | −0.21664 | −0.02883 | −0.03078 | −0.10494 | −0.25485 | −0.14397 | −0.283 | 177 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.419355 | 4.62E−05 | 2.119685 | 0.125 | 0.300485 | 0.395381 | 0.528512 | 0.387476 | 0.502179 | 0.384927 | −0.16869 | −0.04466 | 0.018181 | −0.0746 | 0.090135 | 0.105169 | 178 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.548387 | 5.57E−05 | 1.257205 | 0.125 | −0.4191 | −0.37925 | −0.30924 | −0.2456 | −0.15762 | −0.1402 | −0.10164 | −0.02883 | −0.13743 | −0.08548 | −0.17669 | −0.08919 | 179 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.612903 | 8.80E−05 | 11.462124 | 0.125 | 0.067581 | −0.1046 | −0.13432 | −0.25393 | −0.10572 | −0.17983 | −0.16284 | −0.07596 | −0.36655 | −0.30023 | −0.38417 | −0.35078 | 180 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.548387 | 0.000115 | 1.367948 | 0.125 | −0.3601 | −0.1777 | −0.11702 | 0.012967 | −0.1278 | −0.18971 | −0.0929 | 0.061225 | −0.25176 | −0.07895 | −0.18281 | 0.02708 | 181 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.419355 | 3.63E−05 | 1.448696 | 0.125 | 0.061475 | 0.067816 | 0.055821 | −0.03663 | 0.071788 | 0.048869 | −0.18602 | −0.148 | 0.132289 | −0.02075 | −0.08542 | −0.16483 | 182 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.032258 | 3.61E−05 | 5.567764 | 0.125 | −0.18835 | −0.06379 | −0.04171 | −0.02115 | −0.01577 | 0.021156 | −0.05066 | 0.395094 | −0.16755 | −0.06924 | −0.03915 | 0.091357 | 183 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.612903 | 0.000177 | 1.624337 | 0.125 | −0.22648 | −0.17351 | −0.12019 | −0.15336 | −0.03103 | 0.065522 | −0.03328 | −0.11882 | −0.32627 | −0.03384 | −0.347 | −0.2235 | 184 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.290323 | 2.93E−05 | 1.94499 | 0.125 | 0.077278 | −0.10157 | 0.103537 | −0.15569 | −0.038 | −0.17343 | −0.14834 | −0.04981 | −0.06722 | −0.21766 | −0.14283 | −0.25607 | 185 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 1 | 0.015065 | 0.542637 | 0.125 | 0.42753 | 0.323597 | 0.358658 | 0.201824 | 0.157554 | 0.070724 | −0.05581 | −0.16546 | −0.01433 | 0.025096 | 0.121158 | 0.122619 | 186 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.129032 | 1.36E−05 | 3.248861 | 0.125 | −0.00935 | −0.12791 | 0.054487 | −0.19779 | 0.090943 | −0.08253 | −0.22768 | −0.09703 | −0.06064 | −0.17682 | −0.26072 | −0.3204 | 187 |
| Clostridia;Clostridiales; | | | | | | | | | | | | | | | | | |
| Lachnospiraceae; | | | | | | | | | | | | | | | | | |
| unclassified | | | | | | | | | | | | | | | | | |
| Bacteria;Firmicutes; | 0.483871 | 0.000104 | 1.63865 | 0.25 | −0.08931 | −0.02942 | −0.04925 | −0.12826 | 0.069729 | −0.01757 | −0.00172 | −0.00234 | −0.21794 | −0.18468 | −0.21194 | −0.25553 | 188 |
| Clostridia;Clostridiales; | | | | | | | | | | | | | | | | | |
| Ruminococcaceae; | | | | | | | | | | | | | | | | | |
| Faecalibacterium | | | | | | | | | | | | | | | | | |
| Bacteria;Firmicutes; | 0.967742 | 0.010265 | 1.015259 | 0.25 | 0.193561 | 0.195665 | 0.189642 | 0.215157 | 0.224529 | 0.154243 | −0.26156 | −0.18881 | −0.01638 | 0.028407 | −0.04216 | 0.173162 | 189 |
| Clostridia;Clostridiales; | | | | | | | | | | | | | | | | | |
| Ruminococcaceae; | | | | | | | | | | | | | | | | | |
| Faecalibacterium | | | | | | | | | | | | | | | | | |
| Bacteria;Firmicutes; | 0.451613 | 0.000154 | 1.683517 | 0.125 | 0.193624 | 0.086849 | 0.119643 | 0.042239 | 0.066474 | −0.02091 | −0.01625 | −0.1465 | −0.24864 | −0.13476 | −0.06003 | 0.044337 | 190 |
| Clostridia;Clostridiales; | | | | | | | | | | | | | | | | | |
| Ruminococcaceae; | | | | | | | | | | | | | | | | | |
| Faecalibacterium | | | | | | | | | | | | | | | | | |
| Bacteria;Firmicutes; | 0.354839 | 2.89E−05 | 1.621558 | 0.125 | 0.142334 | 0.364819 | 0.206381 | 0.411396 | 0.261367 | 0.509225 | −0.2727 | −0.17572 | −0.05039 | −0.05581 | −0.2055 | −0.17801 | 191 |
| Clostridia;Clostridiales; | | | | | | | | | | | | | | | | | |
| Ruminococcaceae; | | | | | | | | | | | | | | | | | |
| Faecalibacterium | | | | | | | | | | | | | | | | | |
| Bacteria;Firmicutes; | 0.741935 | 0.000274 | 1.264979 | 0.125 | 0.00304 | −0.07504 | 0.033338 | −0.10972 | −0.00081 | −0.03544 | −0.10296 | −0.06834 | −0.18485 | −0.00443 | −0.25125 | −0.15409 | 192 |
| Clostridia;Clostridiales; | | | | | | | | | | | | | | | | | |
| Ruminococcaceae; | | | | | | | | | | | | | | | | | |
| Faecalibacterium | | | | | | | | | | | | | | | | | |
| Bacteria;Firmicutes; | 0.709677 | 0.000281 | 0.949542 | 0.125 | −0.00346 | −0.08397 | 0.021185 | −0.14721 | 0.02007 | −0.05658 | −0.16393 | −0.08038 | −0.29166 | −0.09343 | −0.30809 | −0.22706 | 193 |
| Clostridia;Clostridiales; | | | | | | | | | | | | | | | | | |
| Ruminococcaceae; | | | | | | | | | | | | | | | | | |
| Faecalibacterium | | | | | | | | | | | | | | | | | |
| Bacteria;Firmicutes; | 0.387097 | 4.41E−05 | 1.754947 | 0.125 | 0.132857 | 0.108247 | 0.180806 | 0.072929 | 0.056647 | 0.061027 | −0.44956 | −0.22972 | −0.12612 | −0.05493 | −0.30607 | −0.20593 | 194 |
| Clostridia;Clostridiales; | | | | | | | | | | | | | | | | | |
| Ruminococcaceae; | | | | | | | | | | | | | | | | | |
| Faecalibacterium | | | | | | | | | | | | | | | | | |
| Bacteria;Firmicutes; | 0.258065 | 3.13E−05 | 3.108913 | 0.125 | 0.416419 | 0.533883 | 0.413607 | 0.522997 | 0.480067 | 0.545399 | −0.04212 | 0.21303 | 0.190959 | 0.076121 | 0.050104 | 0.110056 | 195 |
| Clostridia;Clostridiales; | | | | | | | | | | | | | | | | | |
| Ruminococcaceae; | | | | | | | | | | | | | | | | | |
| Faecalibacterium | | | | | | | | | | | | | | | | | |
| Bacteria;Firmicutes; | 0.741935 | 0.002168 | 1.491187 | 0.5 | 0.179457 | 0.139497 | 0.085681 | 0.020405 | 0.229083 | 0.108124 | −0.11009 | −0.11156 | 0.11497 | 0.074326 | −0.18261 | −0.1953 | 196 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.709677 | 0.000388 | 1.541816 | 0.375 | 0.027151 | 0.106408 | 0.007912 | −0.0462 | 0.119369 | 0.07433 | −0.00267 | −0.02254 | −0.08496 | −0.06796 | −0.12419 | −0.20045 | 197 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.483871 | 0.000139 | 1.291429 | 0.375 | −0.31091 | −0.1324 | −0.35035 | −0.11152 | −0.16841 | 0.02412 | −0.13362 | −0.21336 | −0.0937 | −0.05302 | −0.2805 | −0.38749 | 198 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.548387 | 0.000133 | 1.567384 | 0.125 | −0.05502 | −0.08943 | 0.084841 | 0.028922 | 0.266367 | 0.252599 | −0.19355 | −0.12903 | −0.00906 | 0.206571 | −0.13462 | 0.020875 | 199 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.903226 | 0.015763 | 1.113328 | 0.125 | 0.153652 | 0.044171 | 0.060032 | −0.02892 | 0.046276 | −0.07378 | −0.2654 | −0.27235 | −0.04934 | −0.01585 | −0.155 | −0.02193 | 200 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.419355 | 7.11E−05 | 1.561842 | 0.125 | 0.069525 | −0.0603 | 0.082866 | −0.08494 | 0.113418 | −0.00277 | −0.14286 | −0.08824 | −0.0208 | 0.005534 | −0.16623 | −0.13917 | 201 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.516129 | 6.73E−05 | 1.246947 | 0.125 | 0.070064 | 0.043339 | −0.07151 | −0.06745 | 0.00022 | −0.01152 | −0.29417 | −0.21759 | 0.031418 | 0.049716 | −0.14709 | −0.16589 | 202 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.548387 | 4.75E−05 | 1.173618 | 0.125 | −0.28374 | −0.1894 | −0.21468 | −0.17451 | −0.01741 | −0.02537 | −0.16643 | −0.02712 | 0.17412 | 0.207184 | 0.048103 | 0.035968 | 203 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.870968 | 0.000487 | 1.323221 | 0.125 | 0.272475 | 0.190692 | 0.158848 | 0.11308 | 0.083261 | −0.0149 | −0.19985 | −0.17551 | −0.2086 | −0.18082 | −0.14654 | −0.01645 | 204 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.548387 | 0.000127 | 1.686602 | 0.125 | −0.17901 | −0.10987 | −0.09036 | −0.08613 | 0.13891 | 0.106527 | −0.08803 | 0.024132 | 0.126747 | 0.243626 | −0.10891 | 0.010928 | 205 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.806452 | 0.005824 | 1.049482 | 0.125 | 0.120369 | −0.04772 | 0.144628 | 0.01439 | 0.008761 | −0.06642 | −0.2123 | −0.20036 | 0.3102 | 0.147488 | −0.00914 | −0.05609 | 206 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.16129 | 1.30E−05 | 2.493957 | 0.125 | −0.08018 | −0.0708 | −0.11844 | −0.11347 | −0.12546 | −0.1262 | −0.05175 | −0.08267 | 0.056281 | −0.00042 | 0.039498 | −0.04331 | 207 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | | | | | | | | | | | | | | | | | |
| Clostridia;Clostridiales; | | | | | | | | | | | | | | | | | |
| Ruminococcaceae; | 0.548387 | 0.00024 | 1.835874 | 0.125 | −0.09954 | 0.006999 | −0.03887 | −0.02624 | −0.00063 | 0.082867 | −0.0267 | −0.0787 | −0.04614 | 0.094263 | −0.15649 | −0.10654 | 208 |
| unclassified | | | | | | | | | | | | | | | | | |
| Bacteria;Firmicutes; | | | | | | | | | | | | | | | | | |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.967742 | 0.000335 | 0.767109 | 0.125 | −0.12086 | −0.18734 | −0.09993 | −0.13929 | 0.142466 | 0.135272 | −0.06535 | −0.0321 | 0.081211 | 0.28658 | −0.06683 | 0.023905 | 209 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.322581 | 9.04E−05 | 1.846371 | 0.125 | −0.02591 | 0.018336 | 0.19229 | 0.120675 | 0.150523 | 0.184493 | −0.06997 | 0.181912 | −0.00286 | 0.061743 | 0.029829 | 0.054621 | 210 |
| Clostridia;Clostridiales; | | | | | | | | | | | | | | | | | |
| Ruminococcaceae; | | | | | | | | | | | | | | | | | |
| unclassified | | | | | | | | | | | | | | | | | |
| Bacteria;Firmicutes; | 0.709677 | 0.000755 | 1.115653 | 0.125 | 0.302857 | 0.240238 | 0.210214 | 0.097257 | 0.207308 | 0.189756 | −0.36765 | −0.30894 | 0.1618 | 0.123394 | −0.10401 | −0.20748 | 211 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.709677 | 0.001339 | 1.629547 | 0.5 | −0.37447 | −0.35043 | −0.33703 | −0.38069 | −0.19491 | −0.24313 | −0.05321 | −0.11464 | −0.25227 | −0.25121 | −0.39051 | −0.43513 | 212 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.258065 | 8.65E−05 | 2.329432 | 0.375 | −0.01556 | 0.046806 | −0.12418 | −0.0761 | 0.118582 | 0.127559 | −0.08095 | 0.360556 | −0.05072 | −0.06029 | −0.31071 | −0.16168 | 213 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.612903 | 0.000673 | 1.213608 | 0.375 | 0.313477 | 0.151959 | 0.184798 | 0.033727 | 0.141252 | 0.034758 | −0.19341 | −0.10374 | 0.155841 | 0.204 | 0.003501 | 0.032599 | 214 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.290323 | 0.000135 | 2.770811 | 0.25 | 0.142773 | 0.332545 | 0.07598 | 0.441019 | 0.132031 | 0.441371 | −0.26373 | −0.09348 | −0.13409 | 0.067755 | −0.38039 | −0.08167 | 215 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.451613 | 0.000206 | 1.598464 | 0.25 | 0.145061 | −0.00211 | 0.178705 | 0.020149 | 0.191237 | 0.3631 | −0.29338 | −0.17841 | 0.279298 | 0.197028 | 0.060239 | 0.01149 | 216 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.548387 | 0.00184 | 1.63242 | 0.25 | 0.367861 | 0.279472 | 0.377275 | 0.209144 | 0.1618 | 0.15842 | −0.2081 | −0.20402 | 0.084396 | 0.163313 | 0.000384 | 0.109156 | 217 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.741935 | 0.000388 | 1.334926 | 0.25 | −0.14407 | −0.13913 | 0.029792 | −0.0263 | 0.057571 | 0.059804 | −0.27625 | −0.10623 | −0.06107 | 0.171575 | −0.06447 | 0.064032 | 218 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.83871 | 0.000429 | 1.278232 | 0.125 | −0.27938 | −0.17276 | −0.19313 | −0.06347 | −0.07778 | −0.06274 | −0.20813 | −0.22799 | 0.032409 | 0.065958 | −0.19054 | −0.01255 | 219 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.709677 | 0.000538 | 1.520622 | 0.125 | −0.00835 | 0.004644 | 0.23446 | 0.148146 | 0.064386 | 0.009122 | −0.2668 | −0.09891 | 0.069414 | 0.075586 | 0.095405 | 0.15342 | 220 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.387097 | 3.62E−05 | 1.561876 | 0.125 | 0.04858 | 0.037415 | 0.235269 | 0.121899 | 0.162194 | 0.056804 | −0.10701 | 0.169453 | 0.067732 | 0.064443 | 0.006302 | 0.096407 | 221 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0 | 0 | | 0.125 | −0.14119 | 0.111258 | −0.16604 | 0.026682 | −0.11045 | 0.034639 | −0.14141 | −0.04813 | −0.39545 | −0.3133 | −0.40668 | −0.15232 | 222 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.935484 | 0.002485 | 1.137183 | 0.125 | 0.068121 | −0.05004 | 0.104778 | −0.05659 | 0.058124 | −0.0446 | −0.1662 | −0.06931 | −0.026 | −0.04643 | −0.22742 | −0.19131 | 223 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.451613 | 0.000103 | 1.714318 | 0.125 | 0.025361 | 0.009931 | −0.0353 | −0.03506 | −0.08087 | −0.11644 | −0.31449 | −0.20169 | −0.26845 | −0.14602 | −0.24958 | −0.08661 | 224 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.83871 | 0.000614 | 1.302288 | 0.375 | −0.11143 | 0.004787 | −0.00553 | 0.055555 | 0.244611 | 0.185695 | −0.1419 | 0.06837 | 0.173604 | 0.166872 | 0.000818 | 0.111677 | 225 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.774191 | 0.002685 | 1.612531 | 0.375 | −0.25763 | −0.0565 | −0.11423 | 0.004423 | −0.07464 | 0.153216 | −0.44539 | −0.25993 | 0.052645 | 0.222396 | −0.3438 | −0.1533 | 226 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.419355 | 0.00057 | 2.369729 | 0.375 | −0.14402 | −0.22445 | −0.28777 | −0.28821 | −0.17515 | −0.20779 | −0.38627 | −0.27115 | −0.06197 | 0.15814 | −0.40975 | −0.36257 | 227 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.741935 | 0.001023 | 1.451012 | 0.25 | −0.47822 | −0.39723 | −0.42053 | −0.3889 | −0.34533 | −0.31771 | −0.06137 | 0.440496 | −0.14406 | −0.16636 | −0.22509 | −0.20984 | 228 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.516129 | 0.000672 | 2.706569 | 0.25 | −0.11185 | 0.108531 | −0.14298 | 0.003956 | −0.18873 | 0.048235 | −0.12763 | −0.1782 | 0.006968 | −0.05749 | −0.36548 | −0.24931 | 229 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.677419 | 0.001594 | 1.716022 | 0.125 | −0.51653 | −0.42619 | −0.56649 | −0.5108 | −0.55437 | −0.37835 | −0.0757 | −0.08137 | −0.23704 | −0.24314 | −0.38875 | −0.50502 | 230 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.870968 | 0.002065 | 0.9986 | 0.125 | 0.545569 | 0.351829 | 0.506662 | 0.330246 | 0.263816 | 0.154674 | −0.2013 | −0.21988 | 0.076348 | 0.045474 | 0.066563 | 0.110635 | 231 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.548387 | 0.001217 | 1.609717 | 0.125 | −0.30021 | −0.22775 | −0.18241 | −0.1933 | 0.000509 | −0.0654 | −0.22295 | −0.09565 | 0.148921 | 0.054337 | −0.17048 | −0.16471 | 232 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.806452 | 0.000295 | 1.206449 | 0.125 | 0.263267 | 0.247716 | 0.259037 | 0.180566 | 0.317574 | 0.233923 | −0.21508 | −0.18647 | 0.056436 | 0.127214 | 0.001691 | 0.090899 | 233 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.16129 | 1.57E−05 | 2.632074 | 0.125 | −0.36846 | −0.16461 | −0.16894 | −0.06382 | −0.05224 | 0.000456 | −0.31201 | −0.05411 | 0.057704 | 0.005639 | −0.15182 | 0.037542 | 234 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.806452 | 0.000859 | 1.41356 | 0.125 | 0.02834 | −0.00734 | 0.034418 | 0.017 | 0.073005 | 0.031943 | −0.05071 | −0.00537 | −0.08086 | −0.22854 | −0.1329 | −0.16676 | 235 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.032258 | 3.61E−06 | 5.567764 | 0.125 | −0.03496 | −0.19653 | −0.2879 | −0.33565 | −0.30898 | −0.32763 | −0.07081 | −0.18077 | −0.20043 | −0.26422 | −0.21302 | −0.29889 | 236 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.612903 | 0.000111 | 1.482324 | 0.125 | 0.468222 | 0.318421 | 0.50459 | 0.299925 | 0.395537 | 0.200557 | −0.11482 | −0.09057 | 0.090483 | 0.056429 | 0.132272 | 0.214587 | 237 |
| Clostridia;Clostridiales; | | | | | | | | | | | | | | | | | |
| unclassified; | | | | | | | | | | | | | | | | | |
| unclassified | | | | | | | | | | | | | | | | | |
| Bacteria;Firmicutes; | 0.096774 | 0.000535 | 4.069468 | 1 | −0.30504 | 0.3023 | −0.34494 | −0.35884 | −0.09869 | −0.16487 | −0.26693 | −0.11857 | 0.007607 | 0.014106 | −0.38276 | −0.53172 | 238 |
| Erysipelotrichia; | | | | | | | | | | | | | | | | | |
| Erysipelotrichales; | | | | | | | | | | | | | | | | | |
| Erysipelotrichaceae; | | | | | | | | | | | | | | | | | |
| Erysipelotrichaceae_ | | | | | | | | | | | | | | | | | |
| incertae_sedis | | | | | | | | | | | | | | | | | |
| Bacteria;Firmicutes; | 0.032258 | 0.000322 | 5.567764 | 1 | −0.26671 | −0.24161 | −0.36154 | −0.33096 | −0.20448 | −0.17486 | −0.11471 | 0.273789 | −0.21884 | −0.11819 | −0.34678 | −0.25681 | 239 |
| Erysipelotrichia; | | | | | | | | | | | | | | | | | |
| Erysipelotrichales; | | | | | | | | | | | | | | | | | |
| Erysipelotrichaceae; | | | | | | | | | | | | | | | | | |
| unclassified | | | | | | | | | | | | | | | | | |
| Bacteria;Firmicutes; | 0.064516 | 0.001089 | 5.561483 | 1 | −0.15127 | −0.13996 | −0.25389 | −0.32332 | −0.10359 | −0.13808 | −0.16349 | 0.013387 | −0.22101 | −0.04979 | −0.36207 | −0.36058 | 240 |
| Erysipelotrichia; | | | | | | | | | | | | | | | | | |
| Erysipelotrichales; | | | | | | | | | | | | | | | | | |
| Erysipelotrichaceae; | | | | | | | | | | | | | | | | | |
| unclassified | | | | | | | | | | | | | | | | | |
| Bacteria;Firmicutes; | 0.032258 | 3.61E−06 | 5.567764 | 0.125 | −0.18278 | −0.16659 | −0.373 | −0.3186 | −0.35211 | −0.2388 | −0.2728 | −0.0476 | −0.43371 | −0.36256 | −0.56175 | −0.4229 | 241 |
| Erysipelotrichia; | | | | | | | | | | | | | | | | | |
| Erysipelotrichales; | | | | | | | | | | | | | | | | | |
| Erysipelotrichaceae; | | | | | | | | | | | | | | | | | |
| unclassified | | | | | | | | | | | | | | | | | |
| Bacteria;Firmicutes; | 0.032258 | 3.61E−06 | 5.567764 | 0.125 | 0.085823 | −0.02446 | 0.044426 | −0.03319 | 0.201752 | 0.073248 | −0.07462 | 0.061166 | 0.318478 | 0.300349 | −0.12084 | −0.00785 | 242 |
| Erysipelotrichia; | | | | | | | | | | | | | | | | | |
| Erysipelotrichales; | | | | | | | | | | | | | | | | | |
| Erysipelotrichaceae; | | | | | | | | | | | | | | | | | |
| unclassified | | | | | | | | | | | | | | | | | |
| Bacteria;Firmicutes; | 0.032258 | 1.81E−06 | 5.567764 | 0.125 | −0.1615 | −0.14236 | −0.13714 | −0.17392 | −0.00459 | −0.03108 | −0.15672 | −0.10728 | −0.04729 | −0.08936 | −0.18399 | −0.25728 | 243 |
| Erysipelotrichia; | | | | | | | | | | | | | | | | | |
| Erysipelotrichales; | | | | | | | | | | | | | | | | | |
| Erysipelotrichaceae; | | | | | | | | | | | | | | | | | |
| unclassified | | | | | | | | | | | | | | | | | |
| Bacteria;Firmicutes; | 0.870968 | 0.005456 | 0.891732 | 0.25 | 0.160838 | 0.257819 | 0.115357 | 0.293422 | 0.244545 | 0.251379 | −0.15296 | −0.10817 | −0.01106 | −0.02779 | −0.02133 | 0.069463 | 244 |
| Erysipelotrichia; | | | | | | | | | | | | | | | | | |
| Erysipelotrichales; | | | | | | | | | | | | | | | | | |
| unclassified; | | | | | | | | | | | | | | | | | |
| unclassified | | | | | | | | | | | | | | | | | |
| Bacteria;Firmicutes; | 0.032258 | 3.61E−06 | 5.567764 | 0.125 | 0.139987 | 0.092562 | 0.174294 | 0.101751 | 0.291137 | 0.165302 | −0.10679 | 0.406369 | 0.140412 | 0.274315 | −0.07955 | 0.230505 | 245 |
| Erysipelotrichia; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 0.129032 | 7.88E−06 | 2.824929 | 0.125 | 0.271505 | 0.211629 | 0.30861 | 0.227771 | 0.319886 | 0.159632 | −0.04489 | 0.148687 | 0.11901 | −0.06477 | 0.234752 | 0.178664 | 246 |
| Erysipelotrichia; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 0.032258 | 1.81E−05 | 5.567764 | 0.125 | −0.24682 | −0.25939 | −0.21398 | −0.25921 | −0.05914 | −0.15064 | −0.04612 | 0.48308 | −0.18508 | −0.1834 | −0.24513 | −0.14225 | 247 |
| Negativicutes; | | | | | | | | | | | | | | | | | |
| Selenomonadales; | | | | | | | | | | | | | | | | | |
| Veillonellaceae; | | | | | | | | | | | | | | | | | |
| Megamonas | | | | | | | | | | | | | | | | | |
| Bacteria;Firmicutes; | 0.419355 | 0.000139 | 1.847284 | 0.125 | 0.198418 | 0.113563 | 0.274742 | 0.191072 | −0.02002 | −0.03028 | −0.25793 | −0.13985 | −0.20319 | −0.09073 | −0.0042 | 0.012561 | 248 |
| Negativicutes; | | | | | | | | | | | | | | | | | |
| Selenomonadales; | | | | | | | | | | | | | | | | | |
| Veillonellaceae; | | | | | | | | | | | | | | | | | |
| Veillonella | | | | | | | | | | | | | | | | | |
| Bacteria;Firmicutes; | 0.064516 | 1.54E−05 | 3.931105 | 0.125 | −0.25701 | −0.30753 | −0.20678 | −0.28625 | −0.05115 | −0.16661 | −0.02117 | 0.247279 | −0.23212 | −0.29352 | −0.28065 | −0.19229 | 249 |
| Negativicutes; | | | | | | | | | | | | | | | | | |
| Selenomonadales; | | | | | | | | | | | | | | | | | |
| Veillonellaceae; | | | | | | | | | | | | | | | | | |
| unclassified | | | | | | | | | | | | | | | | | |
| Bacteria;Firmicutes; | 0.064516 | 1.38E−05 | 4.060778 | 0.125 | −0.30371 | −0.20323 | −0.26518 | −0.18886 | −0.11021 | −0.09435 | −0.03797 | 0.27934 | −0.2352 | −0.26829 | −0.25266 | −0.09689 | 250 |
| Negativicutes; | | | | | | | | | | | | | | | | | |
| Selenomonadales; | | | | | | | | | | | | | | | | | |
| Veillonellaceae; | | | | | | | | | | | | | | | | | |
| unclassified | | | | | | | | | | | | | | | | | |
| Bacteria;Firmicutes; | 0.032258 | 7.23E−06 | 5.567764 | 0.125 | −0.33685 | −0.19613 | −0.46088 | −0.31736 | −0.43964 | −0.23812 | −0.06765 | −0.00668 | −0.45739 | −0.32361 | −0.4117 | −0.36106 | 251 |
| Negativicutes; | | | | | | | | | | | | | | | | | |
| Selenomonadales; | | | | | | | | | | | | | | | | | |
| Veillonellaceae; | | | | | | | | | | | | | | | | | |
| unclassified | | | | | | | | | | | | | | | | | |
| Bacteria;Firmicutes; | 0.032258 | 1.08E−05 | 5.567764 | 0.125 | −0.38361 | −0.38068 | −0.24358 | −0.3552 | −0.15867 | −0.22671 | −0.01751 | 0.062516 | −0.28917 | −0.32873 | −0.16059 | −0.26967 | 252 |
| Negativicutes; | | | | | | | | | | | | | | | | | |
| Selenomonadales; | | | | | | | | | | | | | | | | | |
| unclassified; | | | | | | | | | | | | | | | | | |
| unclassified | | | | | | | | | | | | | | | | | |
| Bacteria;Firmicutes; | 0.322581 | 0.001256 | 2.408696 | 0.75 | −0.05919 | −0.00924 | −0.17322 | −0.12734 | 0.00073 | 0.101255 | −0.42821 | −0.25952 | −0.16083 | −0.07403 | −0.50076 | −0.49403 | 253 |
| unclassified; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 0.193548 | 0.000304 | 2.847147 | 0.75 | −0.22228 | −0.20543 | −0.33161 | −0.34946 | −0.08242 | −0.14896 | −0.21489 | 0.008442 | 0.002201 | −0.03983 | −0.41358 | −0.49597 | 254 |
| unclassified; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 0.548387 | 0.000869 | 1.42493 | 0.625 | 0.046961 | −0.10367 | −0.12393 | −0.2732 | −0.17536 | −0.26542 | −0.11649 | −0.04197 | −0.18977 | −0.27053 | −0.25058 | −0.32853 | 255 |
| unclassified; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 1 | 0.003154 | 0.949678 | 0.25 | −0.42918 | −0.2155 | −0.23469 | −0.06398 | −0.21579 | −0.06171 | −0.25016 | −0.23979 | 0.079717 | 0.045381 | −0.26984 | −0.11271 | 256 |
| unclassified; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 0.032258 | 1.81E−06 | 5.567764 | 0.25 | −0.21607 | −0.1738 | −0.31638 | −0.34147 | −0.07646 | −0.14077 | −0.22114 | 0.029062 | −0.0764 | −0.09902 | −0.41567 | −0.49992 | 257 |
| unclassified; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 0.096774 | 0.000195 | 3.701467 | 0.25 | 0.220115 | 0.241427 | 0.044574 | 0.087721 | −0.11815 | −0.05125 | −0.15395 | −0.04911 | −0.22035 | −0.10888 | −0.18664 | 0.82106 | 258 |
| unclassified; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 0.096774 | 4.40E−05 | 4.465898 | 0.25 | 0.244931 | 0.068161 | 0.274107 | 0.180853 | 0.277429 | 0.344111 | −0.32135 | −0.22615 | 0.088586 | 0.293416 | −0.00264 | 0.079615 | 259 |
| unclassified; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 0.903226 | 0.002936 | 1.267472 | 0.25 | −0.34339 | −0.2559 | −0.2999 | −0.21725 | −0.36105 | −0.29772 | −0.16093 | −0.20029 | −0.02211 | 0.003814 | −0.24502 | −0.12248 | 260 |
| unclassified; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 0.548387 | 0.000206 | 1.262493 | 0.125 | −0.16845 | −0.15574 | −0.32822 | −0.31808 | −0.23818 | −0.26545 | −0.2505 | −0.24954 | −0.09359 | −0.05009 | −0.27915 | −0.29323 | 261 |
| unclassified; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 0.322581 | 4.60E−05 | 2.041864 | 0.125 | −0.40227 | −0.34019 | −0.16075 | −0.14994 | −0.07608 | −0.10751 | −0.26335 | −0.20658 | 0.138209 | 0.058333 | −0.20879 | −0.11531 | 262 |
| unclassified; | | | | | | | | | | | | | | | | | |
| unclassified; | | | | | | | | | | | | | | | | | |
| unclassified;unclassified | | | | | | | | | | | | | | | | | |
| Bacteria;Firmicutes; | 0.258065 | 0.000172 | 4.521821 | 0.125 | −0.34236 | −0.24955 | −0.34664 | −0.36227 | −0.38027 | −0.27792 | −0.02877 | 0.019024 | −0.20069 | −0.2959 | −0.31119 | −0.3847 | 263 |
| unclassified; | | | | | | | | | | | | | | | | | |
| unclassified; | | | | | | | | | | | | | | | | | |
| unclassified;unclassified | | | | | | | | | | | | | | | | | |
| Bacteria;Proteobacteria; | 0.83871 | 0.003592 | 1.257042 | 0.125 | −0.04718 | 0.034776 | 0.024908 | 0.176597 | 0.255925 | 0.217825 | −0.11999 | −0.02718 | 0.139239 | 0.040683 | −0.0587 | 0.151284 | 264 |
| Betaproteobacteria; | | | | | | | | | | | | | | | | | |
| Burkholderiales; | | | | | | | | | | | | | | | | | |
| Sutterellaceae; | | | | | | | | | | | | | | | | | |
| Parasutterella | | | | | | | | | | | | | | | | | |
| Bacteria;Proteobacteria; | 0.225806 | 0.00045 | 2.608189 | 0.75 | −0.09016 | −0.09507 | −0.10039 | −0.10898 | 0.138958 | 0.096452 | −0.21078 | 0.294954 | −0.04544 | 0.004084 | −0.26001 | −0.09408 | 265 |
| Betaproteobacteria; | | | | | | | | | | | | | | | | | |
| Burkholderiales; | | | | | | | | | | | | | | | | | |
| Sutterellaceae;Sutterella | | | | | | | | | | | | | | | | | |
| Bacteria;Proteobacteria; | 0.032258 | 3.25E−05 | 5.567764 | 0.5 | −0.14331 | −0.16151 | −0.14634 | −0.18044 | 0.094811 | 0.071706 | −0.12293 | 0.249233 | −0.0321 | 0.049464 | −0.26428 | −0.14238 | 266 |
| Betaproteobacteria; | | | | | | | | | | | | | | | | | |
| Burkholderiales; | | | | | | | | | | | | | | | | | |
| Sutterellaceae;Sutterella | | | | | | | | | | | | | | | | | |
| Bacteria;Proteobacteria; | 0.096774 | 9.26E−05 | 4.239045 | 0.75 | −0.47361 | −0.46846 | −0.5629 | −0.4777 | −0.32482 | −0.28838 | −0.03112 | 0.090144 | −0.07979 | −0.13158 | −0.30365 | −0.24885 | 267 |
| Deltaproteobacteria, | | | | | | | | | | | | | | | | | |
| Desulfovibrionales; | | | | | | | | | | | | | | | | | |
| Desulfovibrionaceae; | | | | | | | | | | | | | | | | | |
| Desulfovibrio | | | | | | | | | | | | | | | | | |
| Bacteria;unclassified; | 0.483871 | 0.000569 | 2.178318 | 0.5 | −0.02166 | 0.15406 | −0.04487 | 0.34805 | 0.169412 | 0.209784 | −0.044 | −0.01351 | 0.215692 | 0.394552 | −0.06479 | 0.07059 | 268 |
| unclassified;unclassified; | | | | | | | | | | | | | | | | | |
| unclassified;unclassified | | | | | | | | | | | | | | | | | |

Donor Pre-Screening and Screening

In various embodiments, human feces are obtained from screened, qualified donors.

In embodiments of the present invention, potential donors are screened via: (1) Initial Preliminary Screen. Prior to enrollment, potential donors (e.g., aged about 18 to about 50) undergo a preliminary screen comprising a subset of questions selected from a Donor Health Questionnaire (DHQ) to assess eligibility and/or (2) In-Person Donor Interview. If the potential donor passes the initial preliminary screen, he/she conducts in-person interview and clinical assessment with a healthcare professional. As part of this interview the potential donor completes informed consent and a donor affidavit attesting to provide true, accurate, and complete information. The DHQ, in-person interview, and clinical assessment determine the potential donor's eligibility as a donor.

The DHQ and clinical assessment identify relevant criteria which would preclude one from being a donor (e.g., temporarily and permanently). Three categories of criteria covered by a DHQ include: (1) Infectious risk factors, e.g., risk for factors for multi-drug resistance organisms (MDROs); high-risk sexual behaviors; social history, including illicit drug use; high-risk travel history (including a 12-month exclusion if a potential donor has traveled to a high-risk or very high-risk area, as defined by current International SOS (ISOS) guidelines); (2) potential microbiome-mediated conditions and general health status, e.g., gastrointestinal comorbidities; metabolic comorbidities; neurological comorbidities; psychiatric comorbidities; chronic pain syndromes; infectious diseases; autoimmune diseases; atopy, asthma and allergies (food and other); malignancy; surgeries/other medical history; current symptoms (including stool habits); medications including antimicrobial therapy; diet; and family history; and (3) pregnancy and breastfeeding status, for potential female donors. In embodiments, the clinical assessment includes, as examples, determination of vital signs including temperature, blood pressure, heart rate, respiratory rate, waist circumference, and body mass index (BMI).

In embodiments, the DHQ is analogous to that used by the Red Cross for screening potential blood donors (with fewer or additional questions, if desired).

Potential donors who are eligible to be donors based upon their DHQ, in-person interview results, and clinical assessment then undergo a series of serological, stool, and nasal swab screens/tests. Serological, stool, and nasal swab testing/screening are performed in conjunction with a diagnostic laboratory, e.g., a Clinical Laboratory Improvement Amendments (CLIA)-certified diagnostic laboratory.

Table 2 provides an overview of exemplary serological, stool, and nasal swab screens/tests conducted as part of the donor screening process of various embodiments. Screening/testing is performed under conditions well-known in the art, such as, by way of a non-limiting example: Hepatitis C may be detected by an immunoassay (IA), Shiga may be detected by enzyme immunoassay (EIA), and Clostridium difficile may be detected by real-time polymerase chain reaction (RT-PCR).

TABLE 2
Exemplary Serological, Stool, and Nasal Swab Screens/Tests

| | Pathogen | |
|---|---|---|
| Serological Testing | HIV 1/2 | |
| | Hepatitis A | |
| | Hepatitis B | |
| | Hepatitis C | |
| | Treponema pallidum | |
| | Strongyloides | |
| Stool Testing | Multi-Drug Resistant | VRE |
| | Organisms | CRE |
| | | FRE |
| | | ESBL |
| | Salmonella spp | |
| | Shigella spp | |
| | Campylobacter spp | |
| | Vibrio spp | |
| | Rotavirus A | |
| | Cryptosporidium spp | |
| | Shiga | |
| | Giardia lamblia | |
| | Adenovirus | |
| | Norovirus | |
| | Clostridium difficile (e.g., a producer of Toxin B) | |
| | Cryptosporidium spp | |
| | Helicobacter pylori | |
| | Ova and parasites | |
| | Cyclospora and Isospora | |
| | Microsporidia | |
| | Bristol Stool Type assessment | |
| Nasal Swab | Multi-Drug Resistant Organisms | VRE |
| | | CRE |
| | | FRE |
| | | MRSA |
| | | ESBL |

VRE = Vancomycin-resistant enterococci; CRE = carbapenem-resistant Enterobacteriaceae; ESBL = Extended-spectrum beta-lactamases; FRE = fluoroquinolone-resistant Enterobacteriaceae.

In some embodiments, a potential donor is excluded if he/she has a positive result in a test/screen for an infectious disease, e.g., caused by one of the pathogens listed in Table 1. In some embodiments, a potential donor who tests positive for HIV-1/2, Hepatitis B, or Hepatitis C is indefinitely excluded from donating.

In some embodiments, a potential donor who tests positive for Hepatitis A, Treponema pallidum, or Strongyloides is deferred from donating until eight weeks after a successful treatment has been completed, symptoms have resolved, and no recurrence of symptoms have occurred.

In some embodiments, a potential donor who tests positive for Adenovirus, Campylobacter spp, Clostridium difficile toxin B, Cryptosporidium spp, Cyclospora and Isospora, Giardia lamblia, Proteus, Morganella, Helicobacter pylori, Microsporidia, Norovirus, Ova and parasites, Salmonella spp, Shiga, Shigella spp, or Vibrio spp, is immediately placed on hold and deferred for eight weeks from symptom resolution, completion of treatment, and no recurrence. Screened donors deferred for eight weeks from symptom resolution, completion of treatment, and no recurrence due to any of the above may undergo a full repeat screen to evaluate for inclusion.

In some embodiments, a potential donor who tests positive for rotavirus is placed immediately on donation hold and undergoes repeat confirmatory testing. If confirmed positive, these donors are ineligible for donation for eight weeks. Screened donors deferred for eight weeks due to rotavirus may undergo a full repeat screen to evaluate for inclusion.

In some embodiments, a potential donor who tests positive for a Multi-Drug Resistant Organism (MDROs), e.g., Vancomycin-resistant Enterococcus (VRE), Carbapenem-resistant enterobacteriaceae (CRE), fluoroquinolone-resistant Enterobacteriaceae (FRE), and Extended-spectrum beta-lactamase (ESBL) is immediately placed on hold and deferred for eight weeks after successful treatment/decolonization with no symptoms or recurrence. Screened donors deferred for eight weeks after successful treatment/decolonization with no symptoms or recurrence due to any of the above may undergo a full repeat screen to evaluate for inclusion.

In some embodiments, a potential donor who tests positive for Methicillin-resistant Staphylococcus aureus (MRSA) is immediately placed on hold and deferred for eight weeks after successful treatment/decolonization with no symptoms or recurrence. Screened donors deferred for eight weeks after successful treatment/decolonization with no symptoms or recurrence due to any of the above may undergo a full repeat screen to evaluate for inclusion.

In some embodiments, potential donors may submit samples for additional screening which may include assays for Liver Function Panel, Alanine Aminotransferase (ALT), Aspartate Aminotransferase (AST), Alkaline Phosphatase (ALP), Albumin, Bilirubin (Total, direct, or indirect), and Complete Blood Count (CBC) with Differential. Donors whose results from these Additional Screening assays are outside the bounds of normal (see, e.g., Table 3) are ineligible to donate stool.

TABLE 3
Exemplary Low and High limit for Complete
Blood Count (CBC) and Hepatic Function Panel (HFP)

| Test | Category | Low | High | Units |
|---|---|---|---|---|
| CBC | WBC | 3.8 | 10.8 | ×10³/μL |
| CBC | RBC | 4.20 | 5.80 | ×10⁶/μL |
| CBC | Hemoglobin | 13.2 | 17.1 | g/dL |
| CBC | Hematocrit | 38.5 | 50.0 | % |
| CBC | MCV | 80 | 100 | fL |
| CBC | MCH | 27.0 | 33.0 | pg |
| CBC | MCHC | 32.0 | 36.0 | g/dL |
| CBC | RDW | 11 | 15 | % |
| CBC | Platelets | 140 | 400 | ×10³/μL |
| CBC | MPV | 7.5 | 11.5 | fL |
| CBC | Absolute Neutrophils | 1500 | 7800 | cells/μL |
| CBC | Absolute Lymphocytes | 850 | 3900 | cells/μL |
| CBC | Absolute Monocytes | 200 | 950 | cells/μL |
| CBC | Absolute Eosinophils | 15 | 500 | cells/μL |
| CBC | Absolute Basophils | 0 | 200 | cells/μL |
| HFP | Protein, Total, Serum | 6.1 | 8.1 | g/dL |
| HFP | Albumin, Serum | 3.6 | 5.1 | g/dL |
| HFP | Bilirubin, Total | 0.2 | 1.2 | mg/dL |
| HFP | Bilirubin, Direct | 0.00 | 0.20 | mg/dL |
| HFP | Bilirubin, Indirect | 0.2 | 1.2 | mg/dL |
| HFP | Alkaline Phosphatase, Serum | 40 | 115 | U/L |
| HFP | AST (SGOT) | 10 | 40 | U/L |
| HFP | ALT (SGPT) | 9 | 46 | U/L |

If the cause of abnormal assay results is found to be either infectious or may otherwise compromise the health of the donor or an FMT recipient, that donor may be excluded from donating stool for clinical use. If the cause of the abnormal reading is determined to be not clinically significant and to pose no threat to an FMT recipient, as examples, the result is an incidental artifact or due to Gilbert's syndrome, then the donor may be considered for enrollment/re-enrollment.

Other screens or tests may also be used to exclude or include potential donors.

In some embodiments, a potential donor may be positive for one or both of Cytomegalovirus (CMV) and Epstein-Barr Virus (EBV). There have not been any reported cases of CMV or EBV infection among those who have received FMT from adult donors (Wang et al., 2016), including a large series of immunocompromised patients (Kelly et al., 2014) and solid organ transplant patients (Fischer et al., 2017).

In some embodiments, a potential donor may be positive for Listeria monocytogenes. In embodiments, donated material and/or serological samples are not tested for L. monocytogenes unless the donor is symptomatic for a L. monocytogenes infection.

In some embodiments, before or after a stool donation event, the pre-screened donor again completes a DHQ. A donor's eligibility will be further evaluated should he/she have any positive responses in this questionnaire. If the donor's responses indicate any changes in health status that involve an exclusion criterion, the donated material is discarded. When the donor's DHQ results do not indicate exclusion, the container and the stool material contained therein is processed.

In some embodiments, a donor may complete an in-person clinical health check around the time of a stool donation to ensure the donor's health. If the donor does not have good/optimal health, the donated material may be discarded.

In some embodiments, a donor is generally of good health and has microbiota consistent with such good health. Often, the donor has not been administered an antibiotic compound within a certain period prior to a stool donation.

In some embodiments, the donor does not have irritable bowel disease (e.g., Crohn's disease and ulcerative colitis), irritable bowel syndrome, celiac disease, colorectal cancer, or a family history of these diseases.

In some embodiments, a donor is selected for the presence of certain genera and/or species that provide increased efficacy of therapeutic compositions containing these genera or species. In some embodiments, a preferred donor donates stool material having a relatively high concentration of spores. In some embodiments, a preferred donor donates stool material comprising spores having increased efficacy.

In some embodiments, a sample of a donated stool material or a donated stool may be used for Additional Screening. Additional Screening may include assays for Liver Function Panel, Alanine Aminotransferase (ALT), Aspartate Aminotransferase (AST), Alkaline Phosphatase (ALP), Albumin, Bilirubin (Total, direct, indirect), and Complete Blood Count (CBC) with Differential. Donors whose results from these Additional Screening assays are outside the bounds of normal (see, e.g., Table 3) the donated material may be discarded.

Other screens or tests may also be used to temporarily or permanently exclude donors.

In some embodiments, a donor who tests positive for Hepatitis A, Treponema pallidum, or Strongyloides is deferred from donating until eight weeks after a successful treatment has been completed, symptoms have resolved, and no recurrence of symptoms have occurred. Impacted donated material will be destroyed. Screened donors deferred for eight weeks from symptom resolution, completion of treatment, and no recurrence due to any of the above may undergo a full repeat screen to evaluate his/her return as a donor.

In some embodiments, a donor who tests positive for Adenovirus, Campylobacter spp, Clostridium difficile toxin B, Cryptosporidium spp, Cyclospora and Isospora, Giardia lamblia, Proteus, Morganella, Helicobacter pylori, Microsporidia, Norovirus, Ova and parasites, Salmonella spp, Shiga, Shigella spp, or Vibrio spp, is immediately placed on hold and deferred for eight weeks from symptom resolution, completion of treatment, and no recurrence. Impacted donated material will be discarded. Screened donors deferred for eight weeks from symptom resolution, completion of treatment, and no recurrence due to any of the above may undergo a full repeat screen to evaluate his/her return as a donor.

In some embodiments, a donor who tests positive for rotavirus will be placed immediately on donation hold and have repeat confirmatory testing performed. If confirmed positive, these donors will have their donated material discarded and will be ineligible for donation for eight weeks. Screened donors deferred for eight weeks due to rotavirus may undergo a full repeat screen to evaluate his/her return as a donor.

A donor who tests positive for a Multi-Drug Resistant Organism (MDROs), e.g., Vancomycin-resistant Enterococcus (VRE), Carbapenem-resistant enterobacteriaceae (CRE), fluoroquinolone-resistant Enterobacteriaceae (FRE) and Extended-spectrum beta-lactamase (ESBL) is immediately placed on hold and deferred for eight weeks after successful treatment/decolonization with no symptoms or recurrence. Impacted donated material will be discarded. Screened donors deferred for eight weeks after successful treatment/decolonization with no symptoms or recurrence due to any of the above may undergo a full repeat screen to evaluate his/her return as a donor.

In some embodiments, a donor who tests positive for Methicillin-resistant Staphylococcus aureus (MRSA) is immediately placed on hold and deferred for eight weeks after successful treatment/decolonization with no symptoms or recurrence. Impacted donated material will be discarded. Screened donors deferred for eight weeks after successful treatment/decolonization with no symptoms or recurrence due to any of the above may undergo a full repeat screen to evaluate his/her return as a donor.

In some embodiments, a donor may be positive for one or both of Cytomegalovirus (CMV) and Epstein-Barr Virus (EBV). There have not been any reported cases of CMV or EBV infection among those who have received FMT from adult donors (Wang et al., 2016), including a large series of immunocompromised patients (Kelly et al., 2014) and solid organ transplant patients (Fischer et al., 2017).

In some embodiments, a donor undergoes a blood test about twenty-one days, e.g., two weeks to a month, or longer, after his/her last donation to account for HIV seroconversion.

In some embodiments, a donor may be positive for Listeria monocytogenes. In embodiments, donated material and/or serological samples are not tested for L. monocytogenes unless the donor is symptomatic for a L. monocytogenes infection.

In embodiments, processing of a donated material begins within six hours of passage of stool material. Elapsed time prior to stool processing may be noted.

In some embodiments, donated material will be assessed using the Bristol stool scale and/or for hematochezia, melena, mucus, and/or steatorrhea. Collection of samples from the donated material may occur within the biosafety cabinet.

Stool below Bristol Type 3 and stool above Bristol Type 5 is discarded.

Stool exhibiting signs of hematochezia, melena, mucus, and/or steatorrhea is discarded.

In some embodiments, donated material is quarantined (i.e., not included in a drug substance and/or not included in a drug product) for a “collection window” of about sixty days, e.g., thirty to ninety days, and until the donor has passed a second DHQ, a second in-person clinical assessment, and/or a second set of serological, stool, and/or nasal swab tests (as described above). See, Table 4.

TABLE 4
Donor Screening/Testing

| | | Testing Time Points | |
|---|---|---|---|
| | | Start of | End of |
| | | collection | collection |
| Parameter | Acceptance Criteria | window | window |
| Questionnaire & | Pass | X | X |
| Interview | | | |
| Serological | Negative for a panel | X | X |
| | of Infectious Diseases | | |
| Stool | Negative for a panel of | X | X |
| | Viruses, Enteric | | |
| | Pathogens, Parasites, etc. | | |
| MDRO | Negative for a panel of Multi- | X | X |
| | Drug Resistant Organisms | | |
| Additional | “Normal” for a Liver | X | X |
| Screening | Function panel and Complete | | |
| | Blood Count & Differential^{b} | | |
| Donor Health | No issues noted that | X | X |
| Questionnaire | involve Exclusion | | |
| (DHQ) completed | Criteria | | |
| at Delivery^{a} | | | |

^{a}In addition to the DHQ, if a donor experiences any abnormal symptoms, including a change in bowel habits or change in other relevant clinical factors (e.g., medicines and medical history) donors should notify to the donation facility immediately. A full health assessment is conducted and if symptoms would lead to stool that may impact the health of an FMT recipient, donation is suspended until an examination of the underlying symptoms is initiated by clinical assessment and/or diagnostic tests on stool and/or blood. The impacted material may be discarded. In the event of transient, self-limiting, mild symptoms, donors may be eligible when symptoms resolve.
^{b}See, Table 3

TABLE 5
Physical Testing Conducted on Donated Material

| Parameter | Acceptance Criteria | Justification |
|---|---|---|
| Bristol Stool Type | Bristol Stool Type must be | Bristol Stool Type of 2, 3, 4, and 5 are considered |
| | Type 3, 4, or 5 | healthy. Types above that range (i.e. Type 6 and |
| | | 7) indicate diarrhea; these Stool Types are not |
| | | processed. |
| | | Stool with a Bristol Stool Type 1 or 2, which indicates |
| | | constipation, may be too rigid or dense for readily |
| | | processing; these Stool Types are not processed. |
| Screening of Stool for | Hematochezia Visually Absent | The presence of fresh blood in stool indicates |
| Hematochezia | | lower gastrointestinal pathology (e.g., |
| | | diverticulosis and inflammatory bowel disease) or, |
| | | less commonly, a brisk upper gastrointestinal |
| | | bleed. Stool with hematochezia is not processed. |
| Screening of Stool for | Melena Visually Absent | The presence of melena in stool indicates upper |
| Melena | | gastrointestinal bleeding (e.g., peptic ulcer |
| | | disease, gastritis, and esophageal varices). Stool |
| | | with melena is not processed. |
| Screening of Stool for | Mucus Visually Absent | Although small amounts of mucus are normal, the |
| Mucus | | presence of mucus in stool potentially indicates |
| | | gastrointestinal pathology (e.g., inflammatory |
| | | bowel disease and malignancy). Stool with mucus |
| | | is processed. |
| Screening of Stool for | Steatorrhea Visually Absent | The presence of steatorrhea in stool indicates fat |
| Steatorrhea | | malabsorption (e.g., pancreatic insufficiency). Stool |
| | | with steatorrhea is not processed. |

In some embodiments, the viability of the microbiota of the donated stool may be confirmed by culturing a sample of the donated stool, an otherwise purified form of the donated stool, a filtrate, a homogenized product, a thawed-frozen intermediate, a pooled material, and/or a drug substance. Methods for culturing microbiota from stool or from stool-derived products are well-known in the art. In some embodiments, microbiota is cultured using the Center for Disease Control (CDC) plate, commonly referred to as “CDC Anaerobe 5% Sheep Blood Agar plate, which allows for the isolation and cultivation of fastidious and slow-growing obligatory anaerobic bacteria, the Bacteroides Bile Esculin Agar (BBE) plate, which is a specific indicator species media for Bacteroides, or GIFU Anaerobic Medium Agar (GAA). In some embodiments, the number of viable, culturable cells within the stool or stool-derived products may be confirmed by the presence of a colony forming unit (CFU) counts, e.g., by the Drop Plate CFU Assay. The diversity of the living microbes in the stool or from stool-derived products may be assayed. The mix of microbes present, or diversity of microbes, is a further measure of the quality of the donated stool and the drug substance.

In some embodiments, the viability of the microbiota of the donated stool may be confirmed by PMAseq; Chu et al., “Using Propodium Monoazide Sequencing (PMA-Seq) to Develop Data-Driven Best Practices in Fecal Microbiota Transplantations.” Open Forum Infect Dis. Oxford University Press; 2015)]. Briefly, this approach provides a high-throughput, culture-independent measure of cell viability.

Pharmaceutical Compositions, Formulations, and Administration

The present invention provides pharmaceutical compositions comprising fresh, frozen, dried, or reconstituted feces from at least one healthy human donor, and optionally, further comprising additional therapeutic agents, in various formulations. The at least one healthy human donor satisfies at least one selection criterion, as described herein.

The present invention also provides pharmaceutical compositions comprising the novel mixtures of bacterial strains, and optionally, further comprising additional therapeutic agents, in various formulations.

The present invention further provides pharmaceutical compositions comprising fresh, frozen, dried, or reconstituted feces from at least one healthy human donor supplemented with novel mixtures of bacterial strains, and optionally, further comprising additional therapeutic agents, in various formulations.

Any pharmaceutical composition described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use.

The formulations comprising the pharmaceutical compositions may conveniently be presented in unit dosage forms. For example, the dosage forms may be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting).

In one embodiment, the pharmaceutical compositions disclosed herein are formulated as a composition adapted for a mode of administration described herein.

In various embodiments, routes of administration include, but are not limited to, oral and rectally. In various embodiments, the administration of the pharmaceutical compositions is oral, naso-gastric, antegrade gastrointestinal, retrograde gastrointestinal, endoscopic, or enemic.

In one embodiment, the pharmaceutical compositions described herein are formulated as a composition adapted for oral administration. Compositions for oral delivery can be in the form of tablets, aqueous or oily suspensions, granules, powders, sprinkles, emulsions, or capsules as examples. Orally administered compositions can comprise one or more agents, for example, sweetening agents such as fructose, aspartame or saccharin;

flavoring agents such as peppermint, oil of wintergreen, or cherry; coloring agents; perfuming agents, to mask an odor of a bacterial mixture; and preserving agents, to provide a pharmaceutically palatable preparation. Moreover, when in capsule, tablet, or pill form, the compositions can be coated to delay disintegration to provide a sustained action over an extended period of time. Selectively permeable membranes surrounding an osmotically active agent driving any pharmaceutical compositions described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, ethacrylic acid and derivative polymers thereof, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In various embodiments, for example, when the pharmaceutical compositions comprise dried feces with or without isolated, purified, and/or cultured bacterial strains, are formulated as solid dosage forms such as tablets, dispersible powders, granules, and capsules. In one embodiment, the pharmaceutical compositions are formulated as a capsule. In another embodiment, the pharmaceutical compositions are formulated as a capsule or tablet. In yet another embodiment, the pharmaceutical compositions are formulated as a soft-gel capsule. In a further embodiment, the pharmaceutical compositions are formulated as a gelatin capsule.

In various embodiments, for example, when the pharmaceutical compositions comprise fresh or reconstituted feces with or without isolated, purified, and/or cultured bacterial strains, formulations suitable for enteral administration include, for example, solutions, suspensions, dispersions, emulsions, and the like. Such formulations may be included in a capsule, e.g., in a gelatin capsule.

Formulations may contain, for example, suspending or dispersing agents.

In various embodiments, the formulations of the invention may additionally comprise a pharmaceutically acceptable carrier or excipient. As one skilled in the art will recognize, the formulations can be in any suitable form appropriate for the desired use and route of administration.

In some dosage forms, the agents described herein are mixed with at least one inert, pharmaceutically acceptable excipient or carrier such as sodium citrate, dicalcium phosphate, and/or (a) fillers or extenders such as starches, lactose, sucrose, glucose, mannitol, silicic acid, microcrystalline cellulose, and Bakers Special Sugar, (b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., (c) humectants such as glycerol, (d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, glyceryl behenate, and mixtures of such excipients. One of skill in the art will recognize that particular excipients may have two or more functions in the oral dosage form. In the case of an oral dosage form, for example, a capsule or a tablet, the dosage form may also comprise buffering agents.

The formulation can additionally include a surface active agent. Surface active agents suitable for use in the present invention include, but are not limited to, any pharmaceutically acceptable, non-toxic surfactant. Classes of surfactants suitable for use in the compositions of the invention include, but are not limited to polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some embodiments, compositions of the invention may comprise one or more surfactants including, but not limited to, sodium lauryl sulfate, polysorbate 20, polysorbate 40, polysorbate 60, polysorbate 80, and triethyl citrate.

The formulation can also contain pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

The formulation can also include one or more application solvents. Some of the more common solvents that can be used to apply, for example, a delayed-release coating composition include isopropyl alcohol, acetone, methylene chloride and the like.

The formulation can also include one or more alkaline materials. Alkaline material suitable for use in compositions of the invention include, but are not limited to, sodium, potassium, calcium, magnesium and aluminum salts of acids such as phosphoric acid, carbonic acid, citric acid and other aluminum/magnesium compounds. In addition, the alkaline material may be selected from antacid materials such as aluminum hydroxides, calcium hydroxides, magnesium hydroxides and magnesium oxide.

Various methods may be used to formulate and/or deliver the agents described herein to a location of interest. For example, the pharmaceutical compositions described herein may be formulated for delivery to the GI tract.

The GI tract includes organs of the digestive system such as mouth, esophagus, stomach, duodenum, small intestine, large intestine (also referred here to as the “colon”) and rectum and includes all subsections thereof (e.g., the small intestine may include the duodenum, jejunum and ileum; the large intestine may include the colon transversum, colon descendens, colon ascendens, colon sigmoidenum and cecum). For example, the bacterial strains and/or pharmaceutical described herein may be formulated for delivery to one or more of the stomach, small intestine, large intestine and rectum and includes all subsections thereof (e.g., duodenum, jejunum and ileum, colon transversum, colon descendens, colon ascendens, colon sigmoidenum and cecum). In some embodiments, the compositions described herein may be formulated to deliver to the upper or lower GI tract. In an embodiment, the bacterial strains and/or pharmaceutical compositions may be administered to a subject, by, for example, directly or indirectly contacting the mucosal tissues of the GI tract.

In various embodiments, the administration the pharmaceutical compositions is into the GI tract via, for example, oral delivery, nasogastral tube, intestinal intubation (e.g., an enteral tube or feeding tube such as, for example, a jejunal tube or gastro-jejunal tube, etc.), direct infusion (e.g., duodenal infusion), endoscopy, colonoscopy, or enema.

For example, in various embodiments, the present invention provides modified-release formulations wherein the formulation releases a substantial amount of the pharmaceutical composition into one or more regions of the GI tract. For example, the formulation may release at least about 60% of the pharmaceutical composition after the stomach and into one or more regions of the GI tract.

In various embodiments, the modified-release formulation of the present invention releases at least 60% of the pharmaceutical composition after the stomach into one or more regions of the intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the the pharmaceutical composition in the intestines.

In various embodiments, the modified-release formulation of the present invention releases at least 60% of the pharmaceutical composition in the small intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the pharmaceutical composition in the small intestine (e.g., one or more of duodenum, jejunum, ileum, and ileocecal junction).

In various embodiments, the modified-release formulation of the present invention releases at least 60% of the pharmaceutical composition in the large intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the pharmaceutical composition in the large intestine (e.g., one or more of cecum, ascending, transverse, descending or sigmoid portions of the colon, and rectum).

In various embodiments, the pharmaceutical composition is formulated for substantially complete delivery prior to the rectum.

In some embodiments, the pharmaceutical composition is formulated for release in the stomach. In other embodiments, the pharmaceutical composition is formulated so as to not substantially release the bacterial strains in the stomach.

In certain embodiments, the modified-release formulation releases the pharmaceutical composition at a specific pH. For example, in some embodiments, the modified-release formulation is substantially stable in an acidic environment and substantially unstable (e.g., dissolves rapidly or is physically unstable) in a near neutral to alkaline environment. In some embodiments, stability is indicative of not substantially releasing while instability is indicative of substantially releasing. For example, in some embodiments, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some embodiments, the present formulations are stable in lower pH areas and therefore do not substantially release in, for example, the stomach. In some embodiments, modified-release formulation is substantially stable at a pH of about 1 to about 4 or lower and substantially unstable at pH values that are greater. In these embodiments, the modified-release formulation does not substantially release in the stomach. In these embodiments, the modified-release formulation substantially releases in the small intestine (e.g., one or more of the duodenum, jejunum, and ileum) and/or large intestine (e.g., one or more of the cecum, ascending colon, transverse colon, descending colon, and sigmoid colon). In some embodiments, modified-release formulation is substantially stable at a pH of about 4 to about 5 or lower and consequentially is substantially unstable at pH values that are greater and therefore is not substantially released in the stomach and/or small intestine (e.g., one or more of the duodenum, jejunum, and ileum). In these embodiments, the modified-release formulation substantially releases in the large intestine (e.g., one or more of the cecum, ascending colon, transverse colon, descending colon, and sigmoid colon). In various embodiments, the pH values recited herein may be adjusted as known in the art to account for the state of the subject, e.g., whether in a fasting or postprandial state.

In some embodiments, the modified-release formulation is substantially stable in gastric fluid and substantially unstable in intestinal fluid and, accordingly, is substantially released in the small intestine (e.g., one or more of the duodenum, jejunum, and ileum) and/or large intestine (e.g., one or more of the cecum, ascending colon, transverse colon, descending colon, and sigmoid colon).

In some embodiments, the modified-release formulation is stable in gastric fluid or stable in acidic environments. These modified-release formulations release about 30% or less by weight of the pharmaceutical composition in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of the of the invention may release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the pharmaceutical composition in the modified-release formulation in gastric fluid with a pH of 4-5, or less or simulated gastric fluid with a pH of 4-5 or less, in about 15, or about 30, or about 45, or about 60, or about 90 minutes. Modified-release formulations of the invention may release about 1%, about 2%, about 3%, about 4%, about 5%, about 6%, about 7%, about 8%, about 9%, or about 10% by weight of the pharmaceutical composition in the modified-release formulation in gastric fluid with a pH of 5 or less, or simulated gastric fluid with a pH of 5 or less, in about 15, or about 30, or about 45, or about 60, or about 90 minutes.

In some embodiments, the modified-release formulation is unstable in intestinal fluid. These modified-release formulations release about 70% or more by weight of the pharmaceutical composition in the modified-release formulation in intestinal fluid or simulated intestinal fluid in about 15, or about 30, or about 45, or about 60, or about 90 minutes. In some embodiments, the modified-release formulation is unstable in near neutral to alkaline environments. These modified-release formulations release about 70% or more by weight of the pharmaceutical composition in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments may release 70% or more by weight of pharmaceutical composition in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In various embodiments, the modified-release formulation of the invention is substantially stable in chyme. For example, there is, in some embodiments, a loss of less about 50% or about 40%, or about 30%, or about 20%, or about 10% of pharmaceutical composition in about 10, or 9, or 8, or 7, or 6, or 5, or 4, or 3, or 2, or 1 hour from administration.

In various embodiments, the modified-release formulations of the present invention are designed for immediate release (e.g., upon ingestion). In various embodiments, the modified-release formulations may have sustained-release profiles, i.e., slow release of the active ingredient(s) in the body (e.g., GI tract) over an extended period of time. In various embodiments, the modified-release formulations may have a delayed-release profile, i.e., not immediately release the active ingredient(s) upon ingestion; rather, postponement of the release of the active ingredient(s) until the composition is lower in the GI tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric coated to delay release of the active ingredient(s) until it reaches the small intestine or large intestine.

In various embodiments, the modified-release formulation of the present invention may utilize one or more modified-release coatings such as delayed-release coatings to provide for effective, delayed yet substantial delivery of the pharmaceutical composition to the GI tract.

In one embodiment, the delayed-release coating includes an enteric agent that is substantially stable in acidic environments and substantially unstable in near neutral to alkaline environments. In an embodiment, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate (CAP), polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, hypromellose (INN) hydroxypropyl methylcellulose (HPMC), shellac or other suitable enteric coating polymers. Similar polymers include Kollicoat® MAE 30 DP and Kollicoat® MAE 100 P. In various embodiments, the enteric agent may be a combination of the foregoing solutions or dispersions. In embodiments, the enteric agent comprises any EUDRAGIT®-type polymer, derivatives thereof, and copolymers thereof. EUDRAGIT® polymers are available from Evonik Industries AG (Essen, Germany).

In certain embodiments, one or more coating system additives are used with the enteric agent. For example, one or more PIasACRYL™ additives may be used as an anti-tacking agent coating additive. Illustrative PIasACRYL™ additives include, but are not limited to PIasACRYL™ HTP20 and PIasACRYL™ T20.

In another embodiment, the delayed-release coating may degrade as a function of time when in aqueous solution without regard to the pH and/or presence of enzymes in the solution. Such a coating may comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings may be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers useful in the present invention include polyvinyl esters, polyvinyl acetals, polyacrylic acid esters, butadiene styrene copolymers, and the like. In one embodiment, colonic delivery is achieved by use of a slowly-eroding wax plug (e.g., various PEGS, including for example, PEG6000).

In some embodiments, an enteric (interior or exterior) coating comprises a polymeric material. Non-limiting examples of suitable polymeric materials include polymethylmethacrylate, poly(N,N-dimethylacrylamide), polyoxamer, polyethylene glycol, polypropylene glycol, polysaccharides (e.g., sucrose, trehalose, glucose, starches such as tapioca and arrowroot, chitosan, alginate, guar gum), polyacrylate, polymethacrylate, polyvinyl alcohol, polyalkylene glycols, polyacrylamide, polyvinylpyrrolidone, polyurethane, polylactide, lactide/glycolide copolymer, polycaprolactone, polydioxanones, polyanhydride, polyhydroxybutyrate, polysiloxane, polytrimethylene carbonate, polyalkylene glycol, and combinations and/or copolymers thereof.

In a further embodiment, the delayed-release coating may be degraded by a microbial enzyme present in the gut flora. In one embodiment, the delayed-release coating may be degraded by a bacteria present in the small intestine. In another embodiment, the delayed-release coating may be degraded by a bacteria present in the large intestine.

Such a coating may comprise a mixture of a first material which is susceptible to attack by colonic bacteria and a second material which has a solubility threshold at about pH 5 or above. The first material may comprise a polysaccharide selected from starch, amylose, amylopectin, chitosan, chondroitin sulfate, cyclodextrin, dextran, pullulan, carrageenan, scleroglucan, chitin, curdulan, and levan. The second material may dissolve in a pH-dependent manner such that it has a “pH threshold” which is the pH below which it is insoluble and at or above which it is soluble. The pH of the surrounding medium triggers dissolution of the second material; thus, little of the second material dissolves below the pH threshold. Once the pH of the surrounding medium reaches (or exceeds) the pH threshold, the second material becomes soluble. In embodiments, the surrounding medium means the medium in the GI tract, such as the gastric juice or intestinal juice or the in vitro equivalent of the medium in the GI tract. The second material may be a film-forming polymeric material such as an acrylate polymer, a cellulose polymer or a polyvinyl-based polymer. Examples of suitable cellulose polymers include cellulose acetate phthalate (“CAP”), cellulose acetate trimellitate (“CAT”), and hydropropylmethylcellulose acetate succinate. Examples of suitable polyvinyl-based polymers include polyvinyl acetate phthalate (“PVAP”). The second material may be a co-polymer of a (meth)acrylic acid and a (meth)acrylic acid 01-4 alkyl ester, for instance, a copolymer of methacrylic acid and methacrylic acid methyl ester. Such a polymer is known as a poly(methacrylic acid/methyl methacrylate) co-polymer. Examples of such co-polymers are usually anionic and not sustained release polymethacrylates. Examples of anionic poly(methacrylic acid/methyl methacrylate) co-polymers include Eudragit® L, Eudragit® S, and Eudragit® FS. The coating may have an additional layer either between the bacterial mixture core and the layer comprising the delayed release composition described above and/or an outer layer coating the delayed release composition layer as described above.

In embodiments, a capsule comprises an interior enteric coating which has hydrophobic properties which prevents or retards the contact of an aqueous phase (e.g., a drug substance of the present disclosure) with the capsule (or capsule material). In embodiments, the interior enteric coating comprises a hydrophobic coating. The hydrophobic coating may comprise a material selected from the group consisting of shellac, zein, polysaccharides, silk, polycaprolactone, oil, pectin, wax, polymers, shellac, and derivatives thereof, and combinations thereof. Non-limiting examples of suitable polysaccharides include alginate, hyaluronic acid, and chitosan. Non-limiting examples of suitable oils include avocado oil, vegetable oil, castor oil, olive oil, jojoba oil, cocoa butter, coconut oil. Non-limiting examples of suitable waxes include beeswax, carnauba wax, and paraffin wax. In some embodiments, the hydrophobic coating is shellac.

An interior enteric coating may be selected and designed such that it protects the capsule (or capsule material) from an aqueous phase. For example, in some embodiments, the interior enteric coating prevents the aqueous phase (e.g., a mixture of bacterial strains of the present disclosure) from contacting the capsule and/or such that the capsule material is not degraded and/or dissolved by the aqueous phase. In some embodiments, the interior enteric coating protects the capsule from the aqueous phase for greater than or equal to 1 day, greater than or equal to 2 days, greater than or equal to 3 days, greater than or equal to 7 days, greater than or equal to 14 days, greater than or equal to 30 days, greater than or equal to 90 days, or greater than or equal to 180 days at room temperature under ambient conditions. In certain embodiments, the interior enteric coating protects the capsule from the aqueous phase for less than or equal to 365 days, less than or equal to 180 days, less than or equal to 90 days, less than or equal to 30 days, less than or equal to 14 days, less than or equal to 7 days, less than or equal to 3 days, or less than or equal to 2 days at room temperature under ambient conditions. Combinations of the above-referenced ranges are possible (e.g., greater than or equal to 1 day and less than or equal to 365 days). Other ranges are also possible. As such, in some embodiments, the capsule is stable at room temperature under ambient conditions for the times listed above (e.g., greater than or equal to 1 day).

In certain embodiments, the interior enteric coating protects the capsule from the aqueous phase (e.g., the interior enteric coating prevents the aqueous phase from contacting the capsule and/or such that the capsule material is not degraded and/or dissolved by the aqueous phase) for greater than or equal to 1 hour, greater than or equal to 2 hours, greater than or equal to 3 hours, greater than or equal to 6 hours, greater than or equal to 12 hours, greater than or equal to 18 hours, greater than or equal to 24 hours, greater than or equal to 48 hours, or greater than or equal to 96 hours at 37° C. In certain embodiments, the interior enteric coating protects the capsule from the aqueous phase for less than or equal to 168 hours, less than or equal to 96 hours, less than or equal to 48 hours, less than or equal to 24 hours, less than or equal to 18 hours, less than or equal to 12 hours, less than or equal to 6 hours, less than or equal to 3 hours, or less than or equal to 2 hours at 37° C. under ambient conditions. Combinations of the above-referenced ranges are possible (e.g., greater than or equal to 1 hour and less than or equal to 168 hours). As such, in certain embodiments, the capsule is stable at 37° C. under ambient conditions for the times listed above (e.g., greater than or equal to 1 hour).

In various embodiments, the modified release formulation is designed for release in the colon. Various colon-specific delivery approaches may be utilized. For example, the modified release formulation may be formulated using a colon-specific drug delivery system (CODES) as described for example, in Li et al., AAPS PharmSciTech (2002), 3(4): 1-9, the entire contents of which are incorporated herein by reference. Drug release in such a system is triggered by colonic microflora coupled with pH-sensitive polymer coatings. For example, the formulation may be designed as a core tablet with three layers of polymer. The first coating is an acid-soluble polymer (e.g., EUDRAGIT E), the outer coating is enteric, along with a hydroxypropyl methylcellulose barrier layer interposed in between. In another embodiment, colon delivery may be achieved by formulating the pharmaceutical composition with specific polymers that degrade in the colon such as, for example, pectin. The pectin may be further gelled or crosslinked with a cation such as a zinc cation. In an embodiment, the formulation is in the form of ionically crosslinked pectin beads which are further coated with a polymer (e.g., EUDRAGIT polymer). Additional colon specific formulations include, but are not limited to, pressure-controlled drug delivery systems (prepared with, for example, ethylcellulose) and osmotic controlled drug delivery systems (i.e., ORDS-CT).

In some embodiments, an enteric (interior or exterior) coating comprises an enteric elastomer. In some embodiments, the enteric elastomer comprises a mixture of two or more polymers with carboxyl functionality such that the two or more polymers form hydrogen bonds with one another and has both enteric and elastic properties. In certain embodiments, the enteric elastomer comprises a first polymer comprising a structure as in Formula (I):

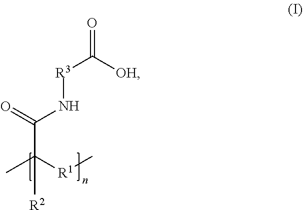

or a pharmaceutically acceptable salt thereof, wherein each R¹ is the same or different and is selected from the group consisting of optionally substituted alkylene, optionally substituted heteroalkylene, optionally substituted arylene, and optionally substituted heteroarylene, each R² is the same or different and is selected from the group consisting of hydrogen, optionally substituted alkyl, and optionally substituted heteroalkyl, each R³ is the same or different and is selected from the group consisting of optionally substituted alkylene and optionally substituted heteroalkylene, n is an integer between 25 and 250,000, and a second polymer comprising a structure as in Formula (II) hydrogen bonded to the first polymer:

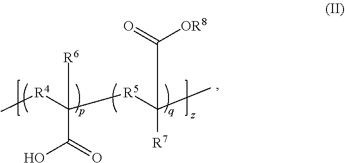

or a pharmaceutically acceptable salt thereof, wherein each R⁴ is the same or different and is selected from the group consisting of optionally substituted alkylene and optionally substituted heteroalkylene, each R⁵ is the same or different and is selected from the group consisting of optionally substituted alkylene and optionally substituted heteroalkylene, each R⁶ is the same or different and is selected from the group consisting of hydrogen, optionally substituted alkyl, and optionally substituted heteroalkyl, each R⁷ is the same or different and is selected from the group consisting of hydrogen, optionally substituted alkyl, and optionally substituted heteroalkyl, each R⁸ is the same or different and is optionally substituted alkyl, p is an integer between 1 and 10, q is an integer between 1 and 10, and z is an integer between 1 and 150,000, provided that (p+q)*z is greater than or equal to 20. Suitable enteric elastomers and methods for making such enteric elastomers are described in more detail in International Patent Publication No. WO2015191922, which is incorporated herein by reference in its entirety for all purposes.

In some embodiments, a capsule comprises a polymeric material. Non-limiting examples of suitable polymeric materials include gelatin, polymethylmethacrylate, poly(N,N-dimethylacrylamide), polyoxamer, polyethylene glycol, polypropylene glycol, polysaccharides (e.g., sucrose, trehalose, glucose, starches such as tapioca and arrowroot, chitosan, alginate, guar gum), polyacrylate, polymethacrylate, polyvinyl alcohol, polyalkylene glycols, polyacrylamide, polyvinylpyrrolidone, polyurethane, polylactide, lactide/glycolide copolymer, polycaprolactone, polydioxanones, polyanhydride, polyhydroxybutyrate, polysiloxane, polytrimethylene carbonate, polyalkylene glycol, and combinations and/or copolymers thereof. In embodiments, the capsule comprises gelatin.

In certain embodiments, the capsule may comprise a bioadherent polymer such as mucin.

Embodiments of dual-coated coated capsules are disclosed in WO2018057747, the contents of which are incorporated by reference in their entirety.

In certain embodiments, the capsule has a particular shape or size. For example, in some cases, the capsule has a shape or size as described in the USP including, but not limited to, #000 capsule, #00 capsule, #0 capsule, #1 capsule, #2 capsule, #3 capsule, #4 capsule, or #5 capsule. Other capsule shapes and/or sizes are also possible.

Formulations for colon specific delivery of the pharmaceutical composition, as described herein, may be evaluated using, for example, in vitro dissolution tests. For example, parallel dissolution studies in different buffers may be undertaken to characterize the behavior of the formulations at different pH levels. Alternatively, in vitro enzymatic tests may be carried out. For example, the formulations may be incubated in fermenters containing suitable medium for bacteria, and the amount of drug released at different time intervals is determined. Drug release studies can also be done in buffer medium containing enzymes or rat or guinea pig or rabbit cecal contents and the amount of drug released in a particular time is determined. In a further embodiment, in vivo evaluations may be carried out using animal models such as dogs, guinea pigs, rats, and pigs. Further, clinical evaluation of colon specific drug delivery formulations may be evaluated by calculating drug delivery index (DDI) which considers the relative ratio of RCE (relative colonic tissue exposure to the drug) to RSC (relative amount of drug in blood i.e., that is relative systemic exposure to the drug). Higher drug DDI indicates better colon drug delivery. Absorption of drugs from the colon may be monitored by colonoscopy and intubation.

In various embodiments, the present formulation provides for substantial uniform dissolution of the pharmaceutical composition in the area of release in the GI tract. In an embodiment, the present formulation minimizes patchy or heterogeneous release of the pharmaceutical composition.

In various embodiments, the present formulations provide for release of multiple doses of the bacterial strains along the GI tract. For example, the composition and/or formulation can release multiple doses of the bacterial strains at different locations along the intestines, at different times, and/or at different pH. The overall release profile of such a formulation may be adjusted using, for example, multiple particle types or multiple layers. For example, in one embodiment, the first dose of the bacterial strains may be formulated for release in, for example, the small intestine (e.g., one or more of duodenum, jejunum, ileum), whereas the second dose is formulated for delayed release in, for example, the large intestines (e.g., one or more of cecum, ascending, transverse, descending or sigmoid portions of the colon, and rectum). In another example, the first dose of the bacterial strains may be formulated for release in, for example, the small intestine (e.g., one or more of duodenum, jejunum, ileum), whereas the second dose is formulated for delayed release in, for example, another part of the small intestine (e.g., one or more of duodenum, jejunum, ileum). In another embodiment, the first dose of the bacterial strains may be formulated for release in, for example, the large intestine (e.g., one or more of cecum, ascending, transverse, descending or sigmoid portions of the colon, and rectum), whereas the second dose is formulated for delayed release in, for example, another part of the large intestine (e.g., one or more of cecum, ascending, transverse, descending or sigmoid portions of the colon, and rectum). In various embodiments, the composition and/or formulation may release at least one dose, at least two doses, at least three doses, at least four doses, or at least five doses of the bacterial strains at different locations along the intestines, at different times, and/or at different pH.

In some embodiments, the bacterial strains described herein are in the form of live, vegetative cells. In some embodiments, the bacterial strains described herein are in the form of spores. In some embodiments, the bacterial strains described herewith are lyophilized. As used herein, “lyophilization” or “freeze drying” refers to the process' of drying a material by first freezing it and then encouraging the ice within it to sublimate in a vacuum environment. By way of non-limiting example, lyophilization can be via methods known in the art, including those described in U.S. Pat. No. 7,799,328, the contents of which are hereby incorporated by reference in their entirety. In some embodiments, lyophilized bacterial strains described herein are placed in an enterically coated soft gel or capsule.

In one aspect, a pharmaceutical composition comprises a lyophilized formulation further comprising a reducing agent. In certain embodiments, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfite, sodium metabisulfite, potassium metabisulfite, Glutathione, Methionine, thioglycerol, and alpha tocopherol.

In one aspect, cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In one aspect, a therapeutic composition comprises a cryoprotectant. As used herein, a “cryoprotectant” refers to a substance that is added to a formulation in order to protect an active ingredient during freezing. In an aspect, a cryoprotectant comprises, consists essentially of, or consists of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof. In an aspect of the present disclosure, a cryoprotectant can be selected from the group comprising 5% Sucrose; 10% Sucrose; 10% Skim milk; 10% Trehalose with 2.5% sucrose; 5% Trehalose with 2.5% sucrose; 5% Mannitol; 5% Mannitol with 0.1% Polysorbate 80; 10% Mannitol; 10% Mannitol with 0.1% Polysorbate 80; 5% Trehalose; 5% Trehalose with 0.1% Polysorbate 80; 10% Trehalose; and 10% Trehaolse with 0.1% Polysorbate 80.

In another aspect, a therapeutic composition comprises a lyoprotectant. As used herein, a “lyoprotectant” refers to a substance that is added to a formulation in order to protect an active ingredient during the drying stage of a lyophilization (also known as freeze-drying) process. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. In one aspect, a lyoprotectant is a non-reducing sugar, such as trehalose or sucrose. In one aspect, a cryoprotectant or a lyoprotectant consists essentially of, or consists of, one or more substances mentioned in this paragraph and the paragraph above.

In one aspect, a cryoprotectant or a lyoprotectant comprise an intracellular agent, e.g., DMSO, Glycerol, or PEG, which penetrates inside the cell preventing the formation of ice crystals that could result in membrane rupture. In another aspect, a cryoprotectant or a lyoprotectant comprise an extracellular agent, e.g., sucrose, trehalose, or dextrose, which does not penetrate into the cell membrane but acts to improve the osmotic imbalance that occurs during freezing.

In one aspect, the present disclosure provides a pharmaceutical composition comprising a lyophilized fecal microbe preparation comprising a lyophilization formulation comprising at least about 12.5% trehalose.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% of trehalose.

In various embodiments, the formulations of the present invention take the form of those as described in one or more of U.S. Pat. Nos. 8,535,713 and 8,9117,77 and US Patent Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In various embodiments, the formulations of the present invention take the form of those as described in International Patent Publication No. WO 2008/135090, the contents of which are hereby incorporated by reference in their entirety.

In various embodiments, the formulations of the present invention take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and US Patent Publication Nos. 2014/0302132; 2014/0227357; 20140088202; 20130287842; 2013/0295188; 2013/0307962; and 20130184290, the contents of which are hereby incorporated by reference in their entirety.

Administration and Dosage

It will be appreciated that the actual dose of the pharmaceutical composition to be administered according to the present invention will vary according to, for example, the particular dosage form and the mode of administration. Many factors that may modify the action of the pharmaceutical composition (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In various embodiments, the dose of the bacterial strains is effective to modulate a patient's microbiome to favor an ecological balance, i.e., treating or preventing a GI disorder described herein.

In various embodiments, the dose of the bacterial strains comprises at least 1×10⁴, 1×10⁵, 1×10⁶, 1×10⁷, 1×10⁸, 1×10⁹, 1×10¹⁰, 1×10¹¹ or greater than 1×10¹¹ colony forming units (CFUs) or bacteria (e.g., germinable bacterial spores).

Individual doses of the pharmaceutical composition can be administered in unit dosage forms (e.g., tablets or capsules) containing, for example, from about 0.01 mg to about 5,000 mg, from about 0.01 mg to about 4,000 mg, from about 0.01 mg to about 3,000 mg, from about 0.01 mg to about 2,000 mg, from about 0.01 mg to about 1,000 mg, from about 0.01 mg to about 950 mg, from about 0.01 mg to about 900 mg, from about 0.01 mg to about 850 mg, from about 0.01 mg to about 800 mg, from about 0.01 mg to about 750 mg, from about 0.01 mg to about 700 mg, from about 0.01 mg to about 650 mg, from about 0.01 mg to about 600 mg, from about 0.01 mg to about 550 mg, from about 0.01 mg to about 500 mg, from about 0.01 mg to about 450 mg, from about 0.01 mg to about 400 mg, from about 0.01 mg to about 350 mg, from about 0.01 mg to about 300 mg, from about 0.01 mg to about 250 mg, from about 0.01 mg to about 200 mg, from about 0.01 mg to about 150 mg, from about 0.01 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg of the active ingredient per unit dosage form, or from about 5 mg to about 80 mg per unit dosage form. For example, a unit dosage form can include about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg of the active ingredient, inclusive of all values and ranges therebetween.

In one embodiment, the pharmaceutical composition is administered at an amount of from about 0.01 mg to about 100 mg daily, an amount of from about 0.01 mg to about 5,000 mg daily, about 0.01 mg to about 4,000 mg daily, about 0.01 mg to about 3,000 mg daily, about 0.01 mg to about 2,000 mg daily, about 0.01 mg to about 1,000 mg daily, from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various embodiments, the pharmaceutical composition is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg inclusive of all values and ranges therebetween.

In some embodiments, a suitable dosage of the pharmaceutical composition is in a range of about 0.01 mg/kg to about 100 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In other embodiments, a suitable dosage of the pharmaceutical composition in a range of about 0.01 mg/kg to about 100 mg/kg of body weight, in a range of about 0.01 mg/kg to about 90 mg/kg of body weight, in a range of about 0.01 mg/kg to about 80 mg/kg of body weight, in a range of about 0.01 mg/kg to about 70 mg/kg of body weight, in a range of about 0.01 mg/kg to about 60 mg/kg of body weight, in a range of about 0.01 mg/kg to about 50 mg/kg of body weight, in a range of about 0.01 mg/kg to about 40 mg/kg of body weight, in a range of about 0.01 mg/kg to about 30 mg/kg of body weight, in a range of about 0.01 mg/kg to about 20 mg/kg of body weight, in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In an aspect, a therapeutic composition provided here comprises a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

As used herein, “Shannon Diversity Index” refers to a diversity index that accounts for abundance and evenness of species present in a given community using the formula:

$$H = - {\sum }_{i = 1}^{R} ( {p}_{i} ) ( ln ( {p}_{i} ) )$$

where H is Shannon Diversity Index, R is the total number of species in the community, and pi is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117pp. In accordance with certain embodiments of the invention, the bacterial strains may be administered, for example, more than once daily, about once per day, about every other day, about every third day, about once a week, about once every two weeks, about once every month, about once every two months, about once every three months, about once every six months, or about once every year.

In one aspect, the present disclosure provides a method for treating a disorder in a subject in need thereof, where the method comprises administering to the subject a pharmaceutically active dose of a therapeutic composition described herein. In one aspect, the present disclosure provides a method for treating a disorder in a subject in need thereof, where the method comprises administering daily to the subject a pharmaceutically active dose of a therapeutic composition described herein. In one aspect, a therapeutic composition is administered to a patient in need thereof at least once daily for at least two consecutive days. In one aspect, a therapeutic composition is administered at least once daily for at least 3, 4, 5, 6, 7, 8, 9, 10, 11, 12, 13, 14, or 15 consecutive days. In another aspect, a therapeutic composition is administered at least once daily for at least 1, 2, 3, 4, 5, 6, 7, 8, 9, 10, 11, or 12 consecutive weeks. In another aspect, a therapeutic composition is administered at least twice, three times, four times, or five times per week for at least 1, 2, 3, 4, 5, 6, 7, 8, 9, 10, 11, or 12 consecutive weeks. In one aspect, a therapeutic composition is administered at least once daily for at most 4, 5, 6, 7, 8, 9, 10, 11, 12, 13, 14, 15, 16, 17, 18, 19, or 20 consecutive days or weeks. In another aspect, a therapeutic composition is administered at least once daily for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to a patient in need thereof at least twice daily for at least two consecutive days. In one aspect, a therapeutic composition is administered at least twice daily for at least 3, 4, 5, 6, 7, 8, 9, 10, 11, 12, 13, 14, or 15 consecutive days. In another aspect, a therapeutic composition is administered at least twice daily for at least 1, 2, 3, 4, 5, 6, 7, 8, 9, 10, 11, or 12 consecutive weeks. In one aspect, a therapeutic composition is administered at least twice daily for at most 4, 5, 6, 7, 8, 9, 10, 11, 12, 13, 14, 15, 16, 17, 18, 19, or 20 consecutive days or week. In another aspect, a therapeutic composition is administered at least twice daily for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to a patient in need thereof at least three times daily for at least two consecutive days. In one aspect, a therapeutic composition is administered at least three times daily for at least 3, 4, 5, 6, 7, 8, 9, 10, 11, 12, 13, 14, or 15 consecutive days. In another aspect, a therapeutic composition is administered at least three times daily for at least 1, 2, 3, 4, 5, 6, 7, 8, 9, 10, 11, or 12 consecutive weeks. In one aspect, a therapeutic composition is administered at least three times daily for at most 4, 5, 6, 7, 8, 9, 10, 11, 12, 13, 14, 15, 16, 17, 18, 19, or 20 consecutive days or weeks. In another aspect, a therapeutic composition is administered at least three times daily for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating a disorder in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota in a lyophilized formulation described herein, where the dose is administered at a dosing schedule of at least once or twice daily for at least three consecutive days or weeks. In another aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating a disorder in a subject in need thereof by administering a pharmaceutical composition described herein, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more fold lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

Additional Therapeutic Agents and Combination Therapy or Co-Formulation

Methods of treatment may comprise administration of bacterial strains (contained in fresh, dried, or reconstituted fecal matter and/or comprising isolated, purified, and/or cultured bacterial strains) of the present invention along with additional therapeutic agents. Co-administration of the additional therapeutic agent and the mixture of bacterial strains may be simultaneous or sequential.

Further; the present formulations may comprise an additional therapeutic agent (e.g., via co-formulation). For example, the additional therapeutic agent and the bacterial strains may be combined into a single formulation.

As used herein, a pharmaceutical composition of the present invention may include or may omit additional therapeutic agents.

In one embodiment, the additional therapeutic agent and the bacterial strains are administered to a subject simultaneously. The term “simultaneously” as used herein, means that the additional therapeutic agent and the bacterial strains are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the bacterial strains can be by simultaneous administration of a single formulation (e.g., a formulation comprising the additional therapeutic agent and the bacterial strains) or of separate formulations (e.g., a first formulation including the additional therapeutic agent and a second formulation including the bacterial strains).

Co-administration does not require the additional therapeutic agents to be administered simultaneously, if the timing of their administration is such that the pharmacological activities of the additional therapeutic agent and the bacterial strains overlap in time. For example, the additional therapeutic agent and the bacterial strains can be administered sequentially. The term “sequentially” as used herein means that the additional therapeutic agent and the bacterial strains are administered with a time separation of more than about 60 minutes. For example, the time between the sequential administration of the additional therapeutic agent and the bacterial strains can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent and the bacterial strains being administered. Either the additional therapeutic agent or the bacterial strains may be administered first.

In a further embodiment, the additional therapeutic agent and the bacterial strains are administered to a subject simultaneously but the release of additional therapeutic agent and the bacterial strains from their respective dosage forms (or single unit dosage form if co-formulated) in the GI tract occurs sequentially.

Co-administration also does not require the additional therapeutic agents to be administered to the subject by the same route of administration. Rather, each additional therapeutic agent can be administered by any appropriate route, for example, parenterally or non-parenterally.

In some embodiments, the additional therapeutic agent is an agent used in the current standard-of-care induction therapies for the pathogenic bacteria that the subject is currently infected with and/or is at risk for being infected with, e.g., one or more anti-inflammatory agents, probiotic agents, prebiotic agents, antidiarrheal agents, analgesics, and antibiotic agents.

In some embodiments, the additional therapeutic agent is an anti-inflammatory agent such as steroidal anti-inflammatory agents or non-steroidal anti-inflammatory agents (NSAIDS). Steroids, particularly the adrenal corticosteroids and their synthetic analogues, are well known in the art. Examples of corticosteroids useful in the present invention include, without limitation, hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that may be used in the present invention, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin. Additional anti-inflammatory agents are described, for example, in U.S. Pat. No. 4,537,776, the entire contents of which are incorporated by reference herein.

In some embodiments, the additional therapeutic agent is a probiotic. Probiotics suitable for use in the present invention include, but are not limited to, Saccharomyces boulardii; Lactobacillus rhamnosus GG; Lactobacillus plantarum 299v; Clostridium butyricum M588; Clostridium difficile VP20621 (non-toxigenic C. difficile strain); combination of Lactobacillus casei, Lactobacillus acidophilus (Bio-K+CL1285); combination of Lactobacillus casei, Lactobacillus bulgaricus, Streptococcus thermophilus (Actimel); combination of Lactobacillus acidophilus, Bifidobacterium bifidum (Florajen3); combination of Lactobacillus acidophilus, Lactobacillus bulgaricus delbrueckii subsp. bulgaricus, Lactobacillus bulgaricus casei, Lactobacillus bulgaricus plantarum, Bifidobacterium longum, Bifidobacterium infantis, Bifidobacterium breve, and Streptococcus salivarius subsp. thermophilus (VSL #3)

The compositions and methods of the present invention may further comprise one or more prebiotics.

A prebiotic is a substrate that is selectively used by a host microorganism to produce a health benefit in a subject/patient. Without wishing to be bound by theory, prebiotics are added to nutritionally supplement bacteria in the microbiome and/or in a microbial composition, e.g., to stimulate the growth or activity of one or more strains of beneficial bacteria. Additionally, the prebiotics may be added to prevent “shock” to bacterial strains subsequent to their isolation or purification, freezing, freeze-drying, spray-drying, reconstitution in solution and the like.

Examples of prebiotics include amino acids, ammonium nitrate, amylose, barley mulch, biotin, carbonate, cellulose, chitin, choline, fructooligosaccharides (FOSs), fructose, galactooligosaccharides (GOSs), glucose, glycerol, heteropolysaccharide, histidine, homopolysaccharide, hydroxyapatite, inulin, isomaltulose, lactose, lactulose, maltodextrins, maltose, mannooligosaccharides, tagatose, nitrogen, oligodextrose, oligofructoses, oligofructose-enriched inulin, oligosaccharides, pectin, phosphate salts, phosphorus, polydextroses, polyols, potash, potassium, sodium nitrate, starch, sucrose, sulfur, sun fiber, tagatose, thiamine, trans-galactooligosaccharides, trehalose, vitamins, a water-soluble carbohydrate, and/or xylooligosaccharides (XOSs).

In embodiments, a prebiotic can be added (e.g., in dry or liquid forms) to a microbial composition of the present invention.

Alternately, or additionally, a prebiotic can be included (e.g., in dry or liquid forms) in a distinct pharmaceutical composition which lacks a microbial composition of the present invention.

A prebiotic may be provided to a subject before, contemporaneously with, and/or after a pharmaceutical composition comprising a microbial composition of the present invention is administered, either in a pharmaceutical composition comprising the microbial composition or in a pharmaceutical composition lacking a microbial composition.

A prebiotic may be provided in a single dose or in multiple doses. When provided as a single composition, the single composition may comprise a single prebiotic or a mixture of prebiotics. When provided in multiple compositions, each composition may comprise a single prebiotic or a mixture of prebiotics.

As examples, when multiple doses are provided, a first composition comprising a prebiotic may include one specific prebiotic, e.g., inulin, and a second composition may include a second specific prebiotic, e.g., pectin. Alternately, a first composition may include a mixture of prebiotics, e.g., inulin and pectin and a second composition may include different mixture of prebiotics, e.g., inulin and a FOS. A first composition may include a mixture of prebiotics and a second composition may include one specific prebiotic.

The amount of prebiotic provided to a subject/patient and/or included in a composition depends on the specific prebiotic, the specific bacterial strain of beneficial bacteria, and/or the disease state of the subject/patientln some embodiments, the additional therapeutic agent is an isolated and purified enteric bacterium that is found in a healthy human GI system.

In some embodiments, the additional therapeutic agent is an antidiarrheal agent. Antidiarrheal agents suitable for use in the present invention include, but are not limited to, DPP-IV inhibitors, natural opioids, such as tincture of opium, paregoric, and codeine, synthetic opioids, such as diphenoxylate, difenoxin and loperamide, bismuth subsalicylate, lanreotide, vapreotide and octreotide, motiln antagonists, COX2 inhibitors like celecoxib, glutamine, thalidomide and traditional antidiarrheal remedies, such as kaolin, pectin, berberine and muscarinic agents.

In some embodiments, the additional therapeutic agent may be an analgesic. Analgesics useful in the compositions and methods of the present invention include, without limitation, morphine, codeine, heroine, methadone and related compounds, thebaine, orpiavine, and their derivatives, buprenorphine, the piperidines, morphinans, benzomorphans, tetrahydroisoquinolines, thiambutanes, benzylamines, tilidine, viminol, nefopam, capsaicin(8-methyl-N-vanillyl-6E-nonenamide), “synthetic” capsaicin(N-vanillylnonamide), and related compounds.

In some embodiments, the additional therapeutic agent is an antibacterial agent, which includes, but is not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In some embodiments, the anti-bacterial agent may be any of the penicillin, cephalosporin, monobactam, and carbapenem antibiotics.

In some embodiments, the additional therapeutic agent includes, but is not limited to, short-chain fatty acids, butyrate, propionate, acetate, IL-2, IL-22, superoxide dismutase (SOD), GLP-2 and analogs, GLP-1, IL-10, IL-27, TGF-β1, TGF-β2, N-acylphosphatidylethanolamines (NAPEs), elafin (also called peptidase inhibitor 3 and SKALP), trefoil factor, melatonin, tryptophan, PGD2, and kynurenic acid, indole metabolites, and other tryptophan metabolites.

For all additional therapeutic agent compositions and methods, targeting to various parts of the GI tract may be employed as described herein.

In various embodiments, the patient of the present methods is undergoing treatment with one or more additional therapeutic agents and, by way of non-limitation, such additional therapeutic agents may disrupt the microbiome.

Methods of Treatment

In various embodiments, the present invention provides methods of modulating a patient's microbiome to provide or restore an ecological balance. For instance, in various embodiments, there is provided methods or diminishing or inhibiting one or more pathogenic bacteria as described elsewhere herein. In various embodiments, the present mixture of bacterial strains augments growth of at least one type of bacteria not detectably present in a patient's GI tract prior to administration and, in various embodiments, which non-pathogenic.

In various embodiments, the present invention provides methods of restoring or enhancing ecological control over gut pathogens or pathobionts in a patient.

In various embodiments, the present invention provides methods of treating or preventing a disease or condition associated with GI dysbiosis, comprising administering an effective amount of a pharmaceutical composition described herein to a subject or a patient need thereof.

In various embodiments, the methods of the invention comprise treating or preventing a microbiome-mediated disorder. Illustrative microbiome-mediated disorder includes, but are not limited to, for example, those found in Table 3 of WO 2014/121298, the entire contents of which are incorporated herein by reference.

In various embodiments, the present invention provides methods of treating a patient suffering from a disease or condition associated with GI dysbiosis. In embodiments, the disease or condition is PSC.

In embodiments, the present invention provides methods in which, following administration of a healthy donor's stool, a PSC patient's microbiome diversity changes towards the diversity present in the donor's stool.

Methods for measuring change and/or improvement in GI tract function can include, but are not limited to: endoscopy for direct examination of epithelium and mucosa; histological evaluation and/or tissue procurement for direct evaluation of structural changes and/or immune biomarkers; urine tests for assessment of permeability with non-absorbable sugars and LPS levels; stool tests for assessment of inflammation and/or microbiota changes (for example by PCR); and/or blood tests for assessment of specific markers, including CD4+ cell counts, Th17 cell counts, and/or LPS levels.

In various embodiments, the methods of the present invention treat or prevent the various GI disorders disclosed herein and/or as known in the art to be a result of gut dysbiosis.

In various embodiments, the methods of the present invention reduce GI immunoactivation and inflammation.

In various embodiments, the methods of the present invention reduce, ameliorate, or eliminate one or more symptom(s) associated with a herein-described disease, disorder, or condition. Exemplary symptoms include, but are not limited to, diarrhea, bloody stool, mouth sores, perianal disease, abdominal pain, abdominal cramping, fever, fatigue, weight loss, iron deficiency, anemia, appetite loss, weight loss, anorexia, delayed growth, delayed pubertal development, and inflammation of the skin, eyes, joints, liver, and bile ducts.

In one aspect, a method comprises administering a therapeutic composition orally, by enema, or via rectal suppository. In one aspect, a pharmaceutical composition is formulated as a geltab, pill, microcapsule, capsule, or tablet. In one aspect, a therapeutic composition is formulated as an enteric coated capsule or microcapsule, acid-resistant capsule or microcapsule, or formulated as part of or administered together with a food, a food additive, a dairy-based product, a soy-based product or a derivative thereof, a jelly, or a yogurt. In another aspect, a therapeutic composition is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

In some embodiments, the terms “patient” and “subject” are used interchangeably. In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish.

In various embodiments, methods of the invention are useful in treatment a human subject. In some embodiments, the human is a pediatric human. In other embodiments, the human is an adult human. In other embodiments, the human is a geriatric human. In other embodiments, the human may be referred to as a patient. In some embodiments, the human is a female. In some embodiments, the human is a male.

In certain embodiments, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

Any aspect or embodiment described herein can be combined with any other aspect or embodiment as disclosed herein.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The terms “one or more”, “at least one”, and the like are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between.

Conversely, the term “no more than” includes each value less than the stated value.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between.

The term “greater than” and the like, is understood to include values greater than the stated by at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between.

A stated range is understood to be any value between and at the limits of the stated range. As examples, a range between 1 and 5 includes 1, 2, 3, 4, and 5; a range between 1 and 10 includes 1, 2, 3, 4, 5, 6, 7, 8, 9, and 10; and a range between 1 and 100 includes 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100.

Unless defined otherwise, all technical and scientific terms used herein have the same meaning as commonly understood by one of ordinary skill in the art to which the invention pertains. Although other probes, compositions, methods, and kits similar, or equivalent, to those described herein can be used in the practice of the present invention, the preferred materials and methods are described herein. It is to be understood that the terminology used herein is for the purpose of describing particular embodiments only, and is not intended to be limiting.

### EXAMPLE

### Example 1: The Present Invention Treats PSC in Human Patients

A fecal microbiota transplant (FMT) study using bacteria obtained from rationally-selected donor was used to treat ten patients with PSC. The ten PSC patients (PSC 101 to PSC 110) were enrolled if they had significantly-elevated blood biomarkers relating to abnormal liver function, but were non-cirrhotic. The ten patients were treated with stool from a single donor via colonoscopy. Patient's stool (for microbiome sequencing) and blood samples (for liver function tests) were collected before FMT and at several time points thereafter, i.e., at one week, at four weeks, at eight weeks, at twelve weeks, and at twenty-four weeks post-FMT.

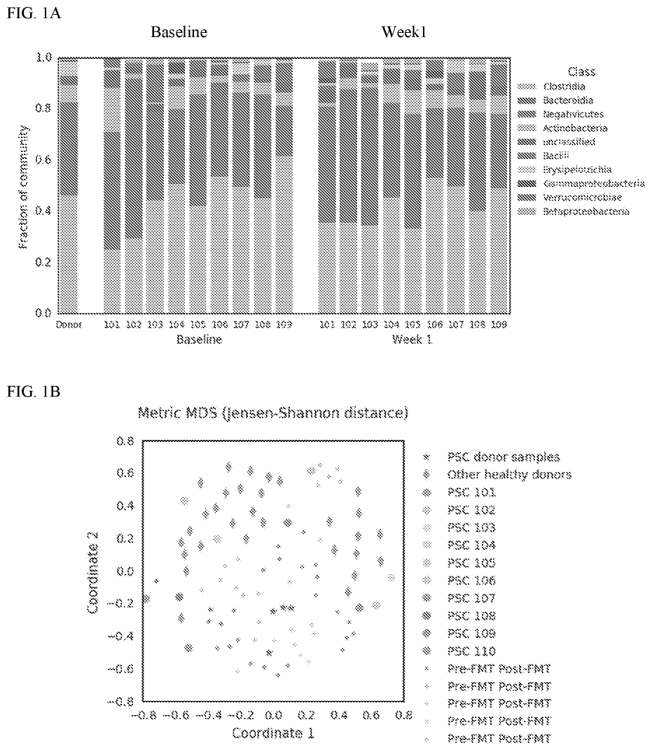

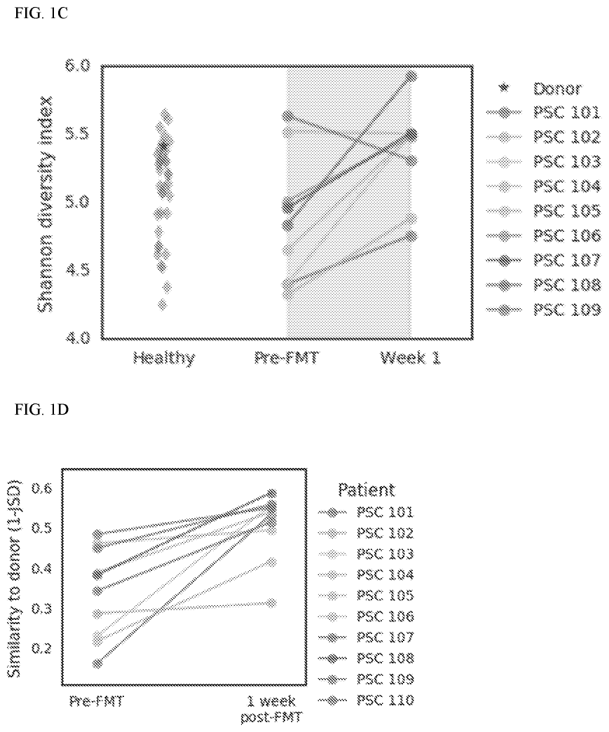

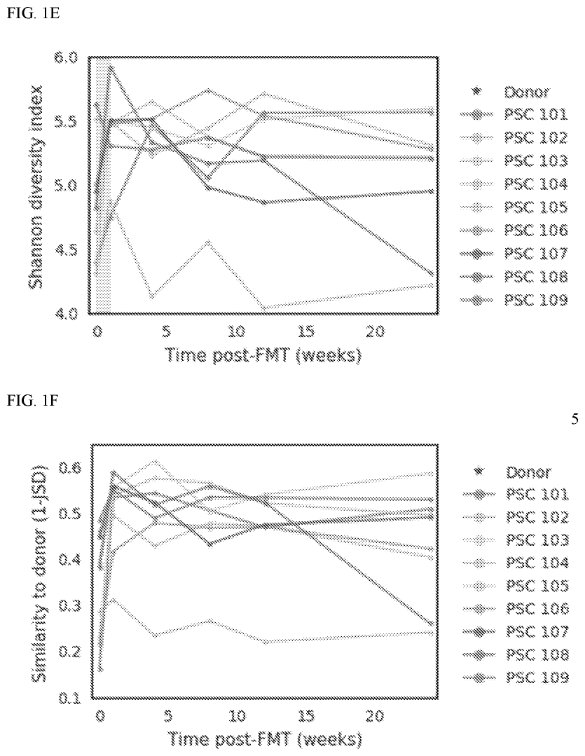

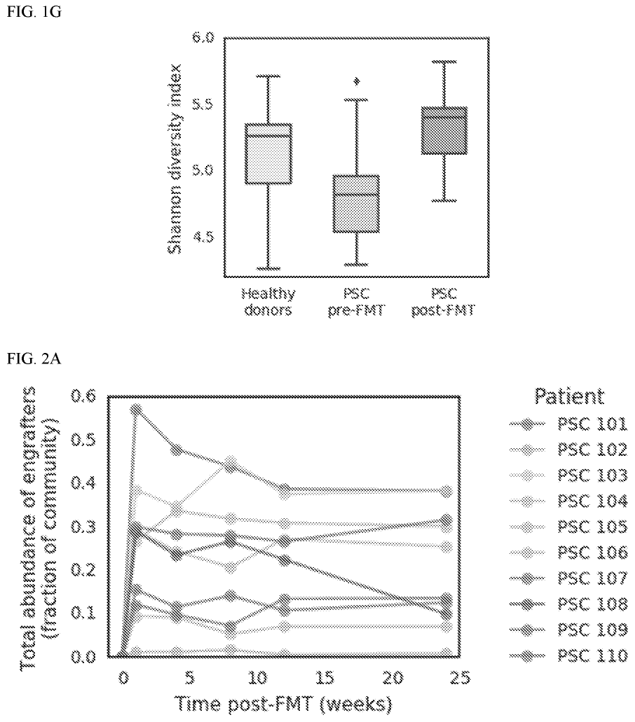

Many patients showed specific changes in their microbiome one week after receiving FMT. As shown in FIG. 1A and FIG. 1B, patients demonstrated measurable changes in the relative proportions of bacterial strains in their microbiome. The majority of patients (7 of 9) had an increase in microbiome diversity (as measured by the Shannon Diversity Index) one week following FMT (FIG. 1C); a different majority of patients had their microbiome diversity change towards the diversity present in the FMT donor (FIG. 1D). The patient's microbiome diversity was sampled up to twenty-four weeks after FMT, with most patient's microbiome diversity remaining relatively stable over the weeks following FMT (FIG. 1E). The majority of patients also maintained their microbiome diversity change relative to the diversity present in the FMT donor (FIG. 1F). As shown in FIG. 1G, when diversity values for the ten patients are pooled, an overall change in diversity amongst the patients following FMT can be seen; moreover, the overall change is seen to be similar to the diversity present in healthy donors.

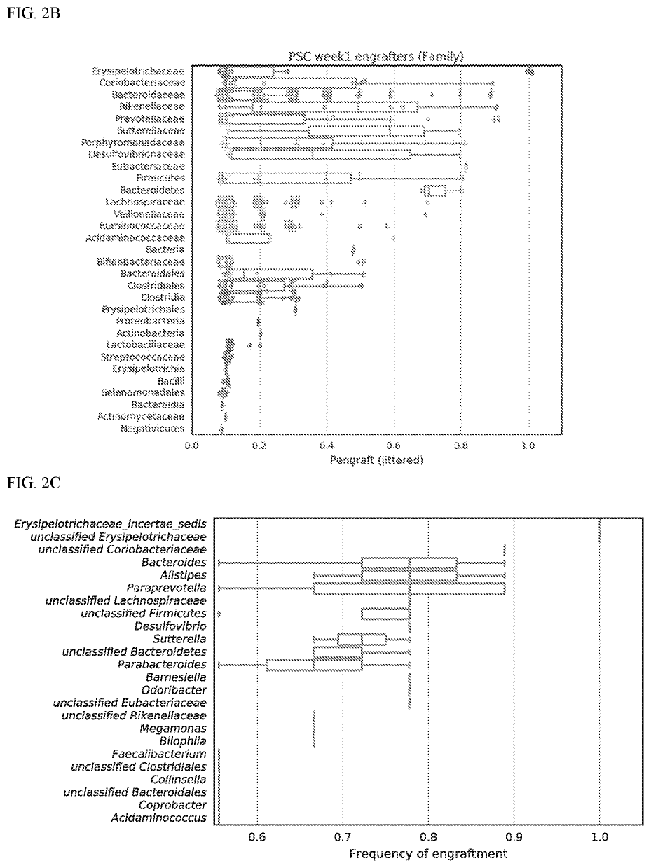

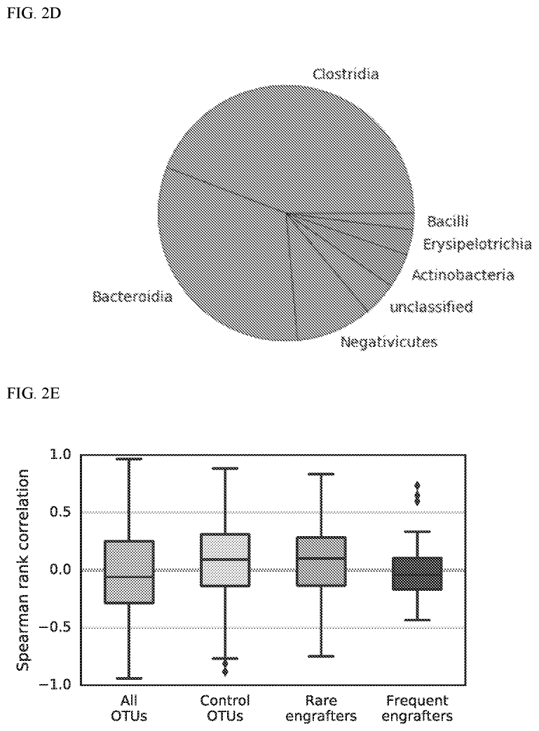

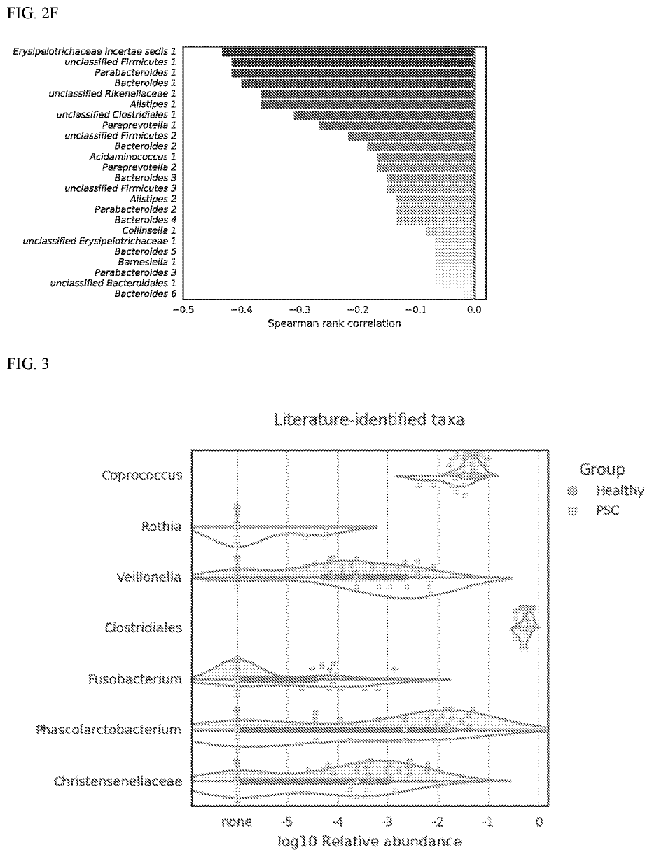

The patient's microbiome was also characterized to identify successful engraftment of the donor microbiome. Here, engraftment is identified as the presence of bacterial strains in the patient following FMT for strains that were present in the donor sample but were absent from the patient before FMT. As shown in FIG. 2A, in the majority of patients, the relative proportion of engrafting strains remained stable over the weeks following FMT.

Engraftment of specific bacterial strains was measured one week after FMT; see, FIG. 2B, which shows clustering by bacterial Family. FIG. 2C further characterizes the data in FIG. 2B by showing the top ten families that were engrafted in patients one week after receiving FMT. FIG. 2D shows the distribution of engrafting bacterial strains at the Class level. FIG. 2E shows that engrafting strains showed increased correlation to liver function biomarker (ALP) improvement compared to control strains (p=0.02). Control strains were defined as OTUs increasing post-FMT but absent from donor samples. Finally, FIG. 2F shows Spearman rank correlation of the twenty-four most frequent engrafting strains that were correlated with ALP decrease.

Following FMT, several patients showed significant improvement in levels of the liver enzymes alkaline phosphatase (ALP), aspartate aminotransferase (AST), and alanine aminotransferase (ALT), which are biomarkers that served as surrogate endpoints in previous FDA trials regarding PSC. Several patients also reported a decrease in extra-intestinal manifestations of disease, such as jaundice, pruritus, and fever.

The above-described experiments identified strains engrafting from the FMT donor to the PSC patients. These strains belong to a diverse set of clades, with some taxa containing multiple strains that consistently engrafted across the majority of patients. The total abundance of engrafting strains remained stable for the duration of the study. Finally, engrafting strains showed increased correlation to improvements in liver function biomarkers. Together, these data suggest that long-term treatment of PSC can be obtained by FMT using stool obtained from a healthy, non-PSC donor.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### REFERENCES

1. Kummen, M., Holm, K., Anmarkrud, J. A., Nygård, S., Vesterhus, M., Høivik, M. L., Trøseid, M., Marschall, H. U., Schrumpf, E., Moum, B. and Røsjø, H., 2017. The gut microbial profile in patients with primary sclerosing cholangitis is distinct from patients with ulcerative colitis without biliary disease and healthy controls. Gut, 66(4), pp. 611-619.
2. Quraishi, M. N., Sergeant, M., Kay, G., Iqbal, T., Chan, J., Constantinidou, C., Trivedi, P., Ferguson, J., Adams, D. H., Pallen, M. and Hirschfield, G. M., 2016. The gut-adherent microbiota of PSC-IBD is distinct to that of IBD. Gut
3. Torres, J., Bao, X., Goel, A., Colombel, J. F., Pekow, J., Jabri, B., Williams, K. M., Castillo, A., Odin, J. A., Meckel, K. and Fasihuddin, F., 2016. The features of mucosa-associated microbiota in primary sclerosing cholangitis. Alimentary pharmacology & therapeutics, 43(7), pp. 790-801.
4. Torres, J., Palmela, C., Brito, H., Bao, X., Ruiqi, H., Moura-Santos, P., Pereira da Silva, J., Oliveira, A., Vieira, C., Perez, K. and ltzkowitz, S. H., 2017. The gut microbiota, bile acids and their correlation in primary sclerosing cholangitis associated with inflammatory bowel disease. United European Gastroenterology Journal.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A pharmaceutical composition comprising a bacterial mixture wherein at least one bacterial strain in the bacterial mixture comprises a 16S V4 sequence that is greater than about 97% identical to the 16S V4 sequence of any one of the operational taxonomic units (OTUs) recited in Table 1.

2. The pharmaceutical composition of claim 1, wherein the 16S V4 sequence of the at least one bacterial strain in the bacterial mixture is greater than about 98% identical to the 16S V4 sequence of any one of the OTUs recited in Table 1.

3. The pharmaceutical composition of claim 1 or claim 2, wherein the 16S V4 sequence of the at least one bacterial strain in the bacterial mixture is greater than about 99% identical to the 16S V4 sequence of any one of the OTUs recited in Table 1.

4. The pharmaceutical composition of any one of claims 1 to 3, wherein the 16S V4 sequence of the at least one bacterial strain in the bacterial mixture is greater than about 99.5% identical to the 16S V4 sequence of any one of the OTUs recited in Table 1.

5. The pharmaceutical composition of any one of claims 1 to 4, wherein the 16S V4 sequence of the at least one bacterial strain in the bacterial mixture is identical to the 16S V4 sequence of any one of the OTUs recited in Table 1.

6. The pharmaceutical composition of any one of claims 1 to 5, wherein the at least one bacterial strain is a commensal bacterial strain.

7. The pharmaceutical composition of any one of claims 1 to 6, wherein the at least one bacterial strain is obtained from one or more human beings.

8. The pharmaceutical composition of claim 7, wherein the one or more human beings are healthy human beings and/or satisfy at least one selection criterion.

9. The pharmaceutical composition of claim 8, wherein the at least one selection criterion comprises a donor having fecal material which lacks or has a low abundance of bacteria that are specifically found in fecal material originating from a PSC patient.

10. The pharmaceutical composition of claim 8, wherein the at least one selection criterion comprises the number of priority bacterial strains and/or their relative abundance in a donor's stool, wherein the priority bacterial strains are identified in Table 1 as having a 16S V4 sequence of one of SEQ ID NO: 1 to SEQ ID NO: 32.

11. The pharmaceutical composition of claim 8, wherein the at least one selection criterion comprises the number of priority clusters and/or their relative abundance in a donor's stool, wherein the priority clusters are identified in Table 1 as having a 16S V4 sequence that is at least 97% identical to one of SEQ ID NO: 10, SEQ ID NO: 13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240.

12. The pharmaceutical composition of claim 11, wherein the presence of a priority cluster and its relative abundance in each donor can be determined by counting the number of sequencing reads with more than 97% identity to the priority clusters' sequences identified in Table 1.

13. The pharmaceutical composition of any one of claims 10 to 12, wherein a donor's stool comprises a least about 5 of the priority bacterial strains and/or the priority clusters identified in Table 1.

14. The pharmaceutical composition of any one of claims 10 to 13 wherein a donor's stool comprises a least about 7 of the priority bacterial strains and/or the priority clusters identified in Table 1.

15. The pharmaceutical composition of any one of claims 10 to 14, wherein a donor's stool comprises a least about 12 of the priority bacterial strains and/or the priority clusters identified in Table 1.

16. The pharmaceutical composition of any one of claims 10 to 15, wherein a donor's stool comprises a relative abundance of priority bacterial strains and/or priority clusters greater than about 0.01% of the total stool bacterial community.

17. The pharmaceutical composition of any one of claims 10 to 16, wherein a donor's stool comprises a relative abundance of priority bacterial strains and/or priority clusters greater than about 0.05% of the total stool bacterial community.

18. The pharmaceutical composition of any one of claims 10 to 17, wherein a donor's stool comprises a relative abundance of priority bacterial strains and/or priority clusters greater than about 0.1% of the total stool bacterial community.

19. The pharmaceutical composition of any one of claims 10 to 18, wherein a donor is selected for being in the top quartile based on the number of priority bacterial strains and/or priority clusters and abundance thereof in their stool relative to other healthy, screened, pathogen-free potential donors.

20. The pharmaceutical composition of any one of claims 10 to 19, wherein a donor is selected for being in the top 10th percentile based on the number of priority bacterial strains and/or priority clusters and abundance thereof in their stool relative to other healthy, screened, pathogen-free potential donors.

21. The pharmaceutical composition of any one of claims 7 to 20, wherein the at least one selection criterion comprises the presence of one or more of the following bacterial strains in a donor's stool: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

22. The pharmaceutical composition of claim 21, wherein the one or more bacterial strains comprises two, three, four, five, six, seven, eight, nine, ten, eleven, or twelve bacterial strains.

23. The pharmaceutical composition of any one of claims 7 to 22, wherein the at least one selection criterion comprises the absence of Primary Sclerosing Cholangitis (PSC) or the absence of symptoms of PSC.

24. The pharmaceutical composition of any one of claims 1 to 23, wherein the at least one bacterial strain is obtained from one human being.

25. The pharmaceutical composition of any one of claims 1 to 24, wherein the at least one bacterial strain is obtained from more than one human being.

26. The pharmaceutical composition of any one of claims 1 to 25, wherein the at least one bacterial strain is isolated and/or purified from its source material prior to forming the bacterial mixture.

27. The pharmaceutical composition of any one of claims 1 to 26, wherein the at least one bacterial strain is cultured prior to forming the bacterial mixture.

28. The pharmaceutical composition of any one of claims 1 to 27, wherein the at least one bacterial strain is not cultured prior to forming the bacterial mixture.

29. The pharmaceutical composition of any one of claims 1 to 28, wherein the at least one bacterial strain is not isolated and/or purified from its source material prior to forming the bacterial mixture.

30. The pharmaceutical composition of claim 29, wherein the at least one bacterial strain is not cultured prior to forming the bacterial mixture.

31. The pharmaceutical composition of claim 30, wherein the source material is fresh, frozen, dried, or reconstituted feces.

32. The pharmaceutical composition of any one of claims 1 to 22, wherein the at least one bacterial strain is obtained from a laboratory stock or bacterial cell bank.

33. The pharmaceutical composition of any one of claims 1 to 31, wherein the at least one bacterial strain is isolated and/or purified from its source material prior to forming the bacterial mixture.

34. The pharmaceutical composition of claim 33, wherein the source material is fresh, frozen, dried, or reconstituted feces.

35. The pharmaceutical composition of any one of claims 32 to 34, wherein the at least one bacterial strain is cultured prior to forming the bacterial mixture.

36. The pharmaceutical composition of any one of claims 1 to 35, wherein the bacterial mixture comprises at least two bacterial strains comprising a 16S V4 sequence that is greater than about 97% identical to the 16S V4 sequence of one of the OTUs recited in Table 1.

37. The pharmaceutical composition of claim 36, wherein the bacterial mixture comprises at least about five bacterial strains, wherein a plurality of the bacterial strains comprises a 16S V4 sequence that is greater than about 97% identical to the 16S V4 sequence of one of the OTUs recited in Table 1.

38. The pharmaceutical composition of claim 37, wherein the bacterial mixture comprises at least about ten bacterial strains, wherein a plurality of the bacterial strains comprises a 16S V4 sequence that is greater than about 97% identical to the 16S V4 sequence of one of the OTUs recited in Table 1.

39. The pharmaceutical composition of claim 38, wherein the bacterial mixture comprises at least about twenty bacterial strains, wherein a plurality of the bacterial strains comprises a 16S V4 sequence that is greater than about 97% identical to the 16S V4 sequence of one of the OTUs recited in Table 1.

40. The pharmaceutical composition of claim 39, wherein the bacterial mixture comprises at least about thirty bacterial strains, wherein a plurality of the bacterial strains comprises a 16S V4 sequence that is greater than about 97% identical to the 16S V4 sequence of one of the OTUs recited in Table 1.

41. The pharmaceutical composition of claim 40, wherein the bacterial mixture comprises at least about forty bacterial strains, wherein a plurality of the bacterial strains comprises a 16S V4 sequence that is greater than about 97% identical to the 16S V4 sequence of one of the OTUs recited in Table 1.

42. The pharmaceutical composition of claim 41, wherein the bacterial mixture comprises at least about fifty bacterial strains, wherein a plurality of the bacterial strains comprises a 16S V4 sequence that is greater than about 97% identical to the 16S V4 sequence of one of the OTUs recited in Table 1.

43. The pharmaceutical composition of any one of claims 1 to 41, wherein the bacterial mixture comprises at least two bacterial strains, wherein each bacterial strain in the bacterial mixture comprises a 16S V4 sequence that is greater than about 97% identical to the 16S V4 sequence of one of the OTUs recited in Table 1.

44. The pharmaceutical composition of claim 43, wherein the bacterial mixture comprises between about five and about one hundred bacterial strains in the bacterial mixture, wherein a plurality of the bacterial strains comprises a 16S V4 sequence that is greater than about 97% identical to the 16S V4 sequence of one of the OTUs recited in Table 1.

45. The pharmaceutical composition of claim 44, wherein the bacterial mixture comprises between about ten and about seventy-five bacterial strains in the bacterial mixture, wherein a plurality of the bacterial strains comprises a 16S V4 sequence that is greater than about 97% identical to the 16S V4 sequence of one of the OTUs recited in Table 1.

46. The pharmaceutical composition of claim 45, wherein the bacterial mixture comprises between about fifteen and about fifty bacterial strains in the bacterial mixture, wherein a plurality of the bacterial strains comprises a 16S V4 sequence that is greater than about 97% identical to the 16S V4 sequence of one of the OTUs recited in Table 1.

47. The pharmaceutical composition of claim 46, wherein the bacterial mixture comprises between about twenty and about forty-five bacterial strains in the bacterial mixture, wherein a plurality of the bacterial strains comprises a 16S V4 sequence that is greater than about 97% identical to the 16S V4 sequence of one of the OTUs recited in Table 1.

48. The pharmaceutical composition of claim 47, wherein the bacterial mixture comprises between about twenty-five and about forty bacterial strains in the bacterial mixture, wherein a plurality of the bacterial strains comprises a 16S V4 sequence that is greater than about 97% identical to the 16S V4 sequence of one of the OTUs recited in Table 1.

49. The pharmaceutical composition of claim 48, wherein the bacterial mixture comprises between about thirty and about thirty-five bacterial strains in the bacterial mixture, wherein a plurality of the bacterial strains comprises a 16S V4 sequence that is greater than about 97% identical to the 16S V4 sequence of one of the OTUs recited in Table 1.

50. The pharmaceutical composition of any one of claims 1 to 41, wherein the bacterial mixture comprises between about five and about one hundred bacterial strains in the bacterial mixture, wherein each bacterial strain comprises a 16S V4 sequence that is greater than about 97% identical to the 16S V4 sequence of one of the OTUs recited in Table 1.

51. The pharmaceutical composition of any one of claims 1 to 50, wherein at least one bacterial strain is included in the bacterial mixture due its greater abundance in the GI tract of a healthy subject relative to its abundance in the GI track of a subject with PSC and/or due to its greater abundance in feces from a healthy subject relative to its abundance in feces from a subject with PSC.

52. The pharmaceutical composition of claim 51, wherein a plurality of bacterial strains is included in the bacterial mixture due to their greater abundance in the GI tract of a healthy subject relative to their abundance in the GI track of a subject with PSC and/or due to their greater abundance in feces from a healthy subject relative to their abundance in feces from a subject with PSC.

53. The pharmaceutical composition of any one of claims 1 to 52, wherein at least one bacterial strain is included in the bacterial mixture due to its ability to engraft in the GI tract of a PSC patient.

54. The pharmaceutical composition of claim 53, wherein a plurality of bacterial strains is included in the bacterial mixture due to their ability to engraft in the GI tract of a PSC patient.

55. The pharmaceutical composition of any one of claims 1 to 54, wherein at least one bacterial strain is included in the bacterial mixture due to its ability to improve levels in the liver biomarker Alkaline Phosphatase (ALP).

56. The pharmaceutical composition of claim 55, wherein a plurality of bacterial strains is included in the bacterial mixture due to their ability to improve levels in the liver biomarker ALP.

57. The pharmaceutical composition of any one of claims 1 to 56, wherein at least one bacterial strain is included in the bacterial mixture due to its ability to reduce inflammation in the bile duct and/or in the liver.

58. The pharmaceutical composition of claim 57, wherein a plurality of bacterial strains is included in the bacterial mixture due to their ability to reduce inflammation in the bile duct and/or in the liver.

59. The pharmaceutical composition of any one of claims 1 to 58, wherein at least one bacterial strain included in the bacterial mixture comprises a 16S V4 sequence that is greater than about 97% identical to one of SEQ ID NO: 1 to SEQ ID NO: 32, SEQ ID NO: 60, SEQ ID NO: 238, or SEQ ID NO: 240.

60. The pharmaceutical composition of claim 59, wherein at least about five bacterial strains included in the bacterial mixture comprise a 16S V4 sequence that is greater than about 97% identical to one of SEQ ID NO: 1 to SEQ ID NO: 32, SEQ ID NO: 60, SEQ ID NO: 238, or SEQ ID NO: 240.

61. The pharmaceutical composition of claim 60, wherein at least about ten bacterial strains included in the bacterial mixture comprise a 16S V4 sequence that is greater than about 97% identical to one of SEQ ID NO: 1 to SEQ ID NO: 32, SEQ ID NO: 60, SEQ ID NO: 238, or SEQ ID NO: 240.

62. The pharmaceutical composition of claim 61, wherein at least about fifteen bacterial strains included in the bacterial mixture comprises a 16S V4 sequence that is greater than about 97% identical to one of SEQ ID NO: 1 to SEQ ID NO: 32, SEQ ID NO: 60, SEQ ID NO: 238, or SEQ ID NO: 240.

63. The pharmaceutical composition of claim 62, wherein at least about twenty bacterial strains included in the bacterial mixture comprises a 16S V4 sequence that is greater than about 97% identical to one of SEQ ID NO: 1 to SEQ ID NO: 32, SEQ ID NO: 60, SEQ ID NO: 238, or SEQ ID NO: 240.

64. The pharmaceutical composition of claim 63, wherein at least about twenty-five bacterial strains included in the bacterial mixture comprise a 16S V4 sequence that is greater than about 97% identical to one of SEQ ID NO: 1 to SEQ ID NO: 32, SEQ ID NO: 60, SEQ ID NO: 238, or SEQ ID NO: 240.

65. The pharmaceutical composition of claim 64, wherein at least about thirty bacterial strains included in the bacterial mixture comprise a 16S V4 sequence that is greater than about 97% identical to one of SEQ ID NO: 1 to SEQ ID NO: 32, SEQ ID NO: 60, SEQ ID NO: 238, or SEQ ID NO: 240.

66. The pharmaceutical composition of claim 65, wherein about thirty-two bacterial strains included in the bacterial mixture comprise a 16S V4 sequence that is greater than about 97% identical to one of SEQ ID NO: 1 to SEQ ID NO: 32, SEQ ID NO: 60, SEQ ID NO: 238, or SEQ ID NO: 240.

67. The pharmaceutical composition of any one of claims 1 to 66, wherein the mixture of bacterial strains comprises one or more of the following bacterial strains: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Delta proteobacteria, Desulfovibrionales, Desulfovibrionaceae, Bilophila; and Bacteria, Proteobacteria, unclassified, unclassified, unclassified, unclassified.

68. The pharmaceutical composition of claim 67, wherein the one or more bacterial strains comprises two, three, four, five, six, seven, eight, nine, ten, eleven, or twelve bacterial strains.

69. The pharmaceutical composition of any one of claims 1 to 68 further comprising a pharmaceutically acceptable excipient.

70. The pharmaceutical composition of any one of claims 1 to 69, wherein the pharmaceutical composition is formulated for oral administration and/or for delivery of the bacterial mixture to an intestine.

71. The pharmaceutical composition of claim 70, wherein the intestine comprises the small intestine or the large intestine.

72. The pharmaceutical composition of claim 71, wherein the intestine comprises the small intestine and the large intestine.

73. The pharmaceutical composition of claim 72, wherein the intestine comprises the large intestine.

74. The pharmaceutical composition of any one of claims 71 to 73, wherein the large intestine comprises the cecum.

75. The pharmaceutical composition of any one of claims 70 to 74, wherein delivery is substantially completed prior to the rectum.

76. The pharmaceutical composition of any one of claims 1 to 75, wherein the pharmaceutical composition is formulated as a capsule.

77. The pharmaceutical composition of claim 76, wherein the capsule comprises a delayed-release coating.

78. The pharmaceutical composition of any one of claims 1 to 77, wherein a plurality of the bacterial strains in the bacterial mixture are live, vegetative cells and/or lyophilized cells.

79. The pharmaceutical composition of any one of claims 1 to 78, wherein a plurality of the bacterial strains in the bacterial mixture are spores.

80. The pharmaceutical composition of any one of claims 1 to 79, wherein a plurality of the bacterial strains in the bacterial mixture are non-pathogenic bacteria.

81. The pharmaceutical composition of any one of claims 1 to 80, wherein each bacterial strain in the bacterial mixture is a non-pathogenic bacterium.

82. The pharmaceutical composition of any one of claims 1 to 81, wherein the pharmaceutical composition is capable of treating or preventing PSC in a subject.

83. The pharmaceutical composition of claim 82, wherein the subject is a human.

84. A method for treating or preventing PSC, comprising administering an effective amount of a pharmaceutical composition of any one of claims 1 to 83 to a subject in need thereof.

85. The method of claim 84, wherein administering an effective amount of the pharmaceutical composition reduces inflammation of the bile duct and/or the liver.

86. A method for treating or preventing Primary Sclerosing Cholangitis (PSC) in a patient in need thereof, comprising administering an effective amount of fresh, frozen, dried, or reconstituted feces from at least one healthy human donor, wherein the at least one healthy human donor satisfies at least one selection criterion.

87. The method of claim 86, wherein the at least one selection criterion comprises a donor having fecal material which lacks or has a low abundance of bacteria that are specifically found in fecal material originating from a PSC patient.

88. The method of claim 86 or claim 87, wherein the at least one selection criterion comprises the number of priority bacterial strains and/or their relative abundance in a donor's stool, wherein the priority bacterial strains are identified in Table 1 as having a 16S V4 sequence of one of SEQ ID NO: 1 to SEQ ID NO: 32.

89. The method of claim 86 or 87, wherein the at least one selection criterion comprises the number of priority clusters and/or their relative abundance in a donor's stool, wherein the priority clusters are identified in Table 1 as having a 16S V4 sequence that is at least 97% identical to one of SEQ ID NO: 10, SEQ ID NO: 13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240.

90. The method of claim 89, wherein the presence of a priority cluster and its relative abundance in each donor can be determined by counting the number of sequencing reads with more than 97% identity to the priority clusters' sequences identified in Table 1.

91. The method of any one of claims 86 to 90, wherein a donor's stool comprises a least about 5 of the priority bacterial strains and/or the priority clusters identified in Table 1.

92. The method of any one of claims 86 to 91, wherein a donor's stool comprises a least about 7 of the priority bacterial strains and/or the priority clusters identified in Table 1.

93. The method of any one of claims 86 to 92, wherein a donor's stool comprises a least about 12 of the priority bacterial strains and/or the priority clusters identified in Table 1.

94. The method of any one of claims 86 to 93, wherein a donor's stool comprises a relative abundance of priority bacterial strains and/or the priority clusters greater than about 0.01% of the total stool bacterial community.

95. The method of any one of claims 86 to 94, wherein a donor's stool comprises a relative abundance of priority bacterial strains and/or the priority clusters greater than about 0.05% of the total stool bacterial community.

96. The method of any one of claims 86 to 95, wherein a donor's stool comprises a relative abundance of priority bacterial strains and/or the priority clusters greater than about 0.1% of the total stool bacterial community.

97. The method of any one of claims 86 to 96, wherein a donor is selected for being in the top quartile based on the number of priority bacterial strains and/or the priority clusters and abundance thereof in their stool relative to other healthy, screened, pathogen-free potential donors.

98. The method of any one of claims 86 to 97, wherein a donor is selected for being in the top 10th percentile based on the number of priority bacterial strains and/or the priority clusters and abundance thereof in their stool relative to other healthy, screened, pathogen-free potential donors.

99. The method of any one of claims 86 to 98, wherein the at least one selection criterion comprises the presence of one or more of the following bacterial strains in a donor's stool: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

100. The method of any one of claims 86 to 99, wherein the one or more bacterial strains comprises two, three, four, five, six, seven, eight, nine, ten, eleven, or twelve bacterial strains.

101. The method of any one of claims 86 to 100, wherein the at least one selection criterion comprises the absence of PSC or the absence of symptoms of PSC.

102. The method of any one of claims 86 to 101, wherein the effective amount of fresh, frozen, dried, or reconstituted feces reduces inflammation of the bile duct and/or the liver.

103. The method of any one of claims 86 to 102, wherein the effective amount of fresh, frozen, dried, or reconstituted feces improves levels of the liver biomarker Alkaline Phosphatase (ALP).

104. The method of any one of claims 86 to 103, wherein the fresh, frozen, dried, or reconstituted feces comprises a substantially complete fecal microbiota obtained from one healthy human donor.

105. The method of any one of claims 86 to 104, wherein the fresh, frozen, dried, or reconstituted feces is obtained from more than one healthy human donor.

106. The method of any one of claims 86 to 105, wherein the fresh, frozen, dried, or reconstituted feces comprises spores and/or live, vegetative cells.

107. The method of any one of claims 86 to 106, wherein the fresh, frozen, dried, or reconstituted feces comprises a plurality of non-pathogenic bacteria.

108. The method of any one of claims 86 to 107, wherein the fresh, frozen, dried, or reconstituted feces comprises a plurality of bacterial strains having greater abundances relative to their abundances in fresh, frozen, dried, or reconstituted feces from a subject with PSC.

109. The method of any one of claims 86 to 108, wherein the fresh, frozen, dried, or reconstituted feces comprises at least one bacterial strain capable of engrafting in the GI tract of a PSC patient.

110. The method of any one of claims 86 to 109, further comprising administering at least one isolated, purified, and/or cultured bacterial strain comprising a 16S V4 sequence that is greater than about 97% identical to the 16S V4 sequence of any one of the operational taxonomic units (OTUs) recited in Table 1.

111. The method of any one of claims 86 to 110, further comprising administering a pharmaceutically acceptable excipient combined with the fresh, frozen, dried, or reconstituted feces.

112. The method of any one of claims 86 to 111, wherein the patient in need thereof is a human.

113. A method for manufacturing a pharmaceutical composition of any one of claims 1 to 85 comprising obtaining at least one bacterial strain comprising a 16S V4 sequence that is greater than about 97% identical to the 16S V4 sequence of any one of the operational taxonomic units (OTUs) recited in Table 1 and formulating the least one bacterial strain into a pharmaceutical composition.

114. The method of claim 113, wherein the at least one bacterial strain is contained in fresh, frozen, dried, or reconstituted feces obtained from one or more healthy human beings who satisfy at least one selection criterion.

115. The method of claim 114, wherein the at least one selection criterion comprises a donor having fecal material which lacks or has a low abundance of bacteria that are specifically found in fecal material originating from a PSC patient.

116. The method of claim 114 or claim 115, wherein the at least one selection criterion comprises the number of priority bacterial strains and/or their relative abundance in a donor's stool, wherein the priority bacterial strains are identified in Table 1 as having a 16S V4 sequence of one of SEQ ID NO: 1 to SEQ ID NO: 32.

117. The method of claim 114 or claim 115, wherein the at least one selection criterion comprises the number of priority clusters and/or their relative abundance in a donor's stool, wherein the priority clusters are identified in Table 1 as having a 16S V4 sequence that is at least 97% identical to one of SEQ ID NO: 10, SEQ ID NO: 13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240.

118. The method of claim 117 wherein the presence of a priority cluster and its relative abundance in each donor can be determined by counting the number of sequencing reads with more than 97% identity to the priority clusters' sequences identified in Table 1.

119. The method of any one of claims 114 to 118, wherein a donor's stool comprises a least about 5 of the priority bacterial strains and/or the priority clusters identified in Table 1.

120. The method of any one of claims 114 to 119, wherein a donor's stool comprises a least about 7 of the priority bacterial strains and/or the priority clusters identified in Table 1.

121. The method of any one of claims 114 to 120, wherein a donor's stool comprises a least about 12 of the priority bacterial strains and/or the priority clusters identified in Table 1.

122. The method of any one of claims 114 to 121, wherein a donor's stool comprises a relative abundance of priority bacterial strains and/or the priority clusters greater than about 0.01% of the total stool bacterial community.

123. The method of any one of claims 114 to 122, wherein a donor's stool comprises a relative abundance of priority bacterial strains and/or the priority clusters greater than about 0.05% of the total stool bacterial community.

124. The method of any one of claims 114 to 123, wherein a donor's stool comprises a relative abundance of priority bacterial strains and/or the priority clusters greater than about 0.1% of the total stool bacterial community.

125. The method of any one of claims 114 to 124, wherein a donor is selected for being in the top quartile based on the number of priority bacterial strains and/or the priority clusters and abundance thereof in their stool relative to other healthy, screened, pathogen-free potential donors.

126. The method of any one of claims 114 to 125, wherein a donor is selected for being in the top 10th percentile based on the number of priority bacterial strains and/or the priority clusters and abundance thereof in their stool relative to other healthy, screened, pathogen-free potential donors.

127. The method of any one of claims 114 to 126, wherein the at least one selection criterion comprises the presence of one or more of the following bacterial strains in a donor's stool: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

128. The method of any one of claims 114 to 127, wherein the one or more bacterial strains comprises two, three, four, five, six, seven, eight, nine, ten, eleven, or twelve bacterial strains.

129. The method of any one of claims 114 to 128, wherein the at least one selection criterion comprises the absence of PSC or the absence of symptoms of PSC.

130. A method for manufacturing a pharmaceutical composition suitable for the treatment of PSC, comprising:
(a) screening a potential human feces donor for the presence of at least one selection criterion;
(b) selecting a potential human feces donor as a human feces donor based upon the presence of the at least one selection criterion;
(c) obtaining feces from the human feces donor; and
(d) formulating the obtained feces into a pharmaceutical composition for administration to a PSC patient.

131. The method of claim 130, wherein the at least one selection criterion comprises the number of priority bacterial strains and/or their relative abundance in a donor's stool, wherein the priority bacterial strains are identified in Table 1 as having a 16S V4 sequence of one of SEQ ID NO: 1 to SEQ ID NO: 32.

132. The method of claim 130, wherein the at least one selection criterion comprises the number of priority clusters and/or their relative abundance in a donor's stool, wherein the priority clusters are identified in Table 1 as having a 16S V4 sequence that is at least 97% identical to one of SEQ ID NO: 10, SEQ ID NO:
13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240.

133. The method of claim 132 wherein the presence of a priority cluster and its relative abundance in each donor can be determined by counting the number of sequencing reads with more than 97% identity to the priority clusters' sequences identified in Table 1.

134. The method of any one of claims 130 to 133, wherein a donor's stool comprises a least about 5 of the priority bacterial strains and/or the priority clusters identified in Table 1.

135. The method of any one of claims 130 to 134, wherein a donor's stool comprises a least about 7 of the priority bacterial strains and/or the priority clusters identified in Table 1.

136. The method of any one of claims 130 to 135, wherein a donor's stool comprises a least about 12 of the priority bacterial strains and/or the priority clusters identified in Table 1.

137. The method of any one of claims 130 to 136, wherein a donor's stool comprises a relative abundance of priority bacterial strains and/or the priority clusters greater than about 0.01% of the total stool bacterial community.

138. The method of any one of claims 130 to 137, wherein a donor's stool comprises a relative abundance of priority bacterial strains and/or the priority clusters greater than about 0.05% of the total stool bacterial community.

139. The method of any one of claims 130 to 138, wherein a donor's stool comprises a relative abundance of priority bacterial strains and/or the priority clusters greater than about 0.1% of the total stool bacterial community.

140. The method of any one of claims 130 to 139, wherein a donor is selected for being in the top quartile based on the number of priority bacterial strains and/or the priority clusters and abundance thereof in their stool relative to other healthy, screened, pathogen-free potential donors.

141. The method of any one of claims 130 to 140, wherein a donor is selected for being in the top 10th percentile based on the number of priority bacterial strains and/or the priority clusters and abundance thereof in their stool relative to other healthy, screened, pathogen-free potential donors.

142. The method of any one of claims 130 to 141, wherein the at least one selection criterion comprises the presence of one or more of the following bacterial strains in a donor's stool: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

143. The method of any one of claims 130 to 142, wherein the one or more bacterial strains comprises two, three, four, five, six, seven, eight, nine, ten, eleven, or twelve bacterial strains.

144. The method of any one of claims 130 to 143, wherein the at least one selection criterion comprises the absence of PSC or the absence of symptoms of PSC.

145. The method of any one of claims 130 to 144, wherein the at least one selection criterion comprises a donor having fecal material which lacks or has a low abundance of bacteria that are specifically found in fecal material originating from a PSC patient.

146. The method of any one of claims 130 to 145, wherein the obtained feces is fresh, frozen, dried, or reconstituted feces.

147. The method of any one of claims 130 to 146, wherein the pharmaceutical composition further comprises at least one bacterial strain that is isolated, purified, and/or cultured.

148. The method of claim of any one of claims 130 to 147, wherein the at least bacterial strain comprises a 16S V4 sequence that is greater than about 97% identical to the 16S V4 sequence of any one of the operational taxonomic units (OTUs) recited in Table 1.

149. The method of any one of claims 130 to 148, wherein the pharmaceutical composition further comprises a pharmaceutically acceptable excipient.

150. A method for manufacturing a pharmaceutical composition suitable for the treatment of PSC, comprising:
(a) screening a plurality of potential human feces donors for the presence of at least one selection criterion;
(b) selecting a plurality of potential human feces donor as human feces donors based upon the presence of the at least one selection criterion;
(c) obtaining feces from the human feces donors; and
(d) formulating the obtained feces into a pharmaceutical composition for administration to a PSC patient.

151. The method of claim 150, wherein the at least one selection criterion comprises a donor having fecal material which lacks or has a low abundance of bacteria that are specifically found in fecal material originating from a PSC patient.

152. The method of claim 150 or claim 151, wherein the at least one selection criterion comprises the number of priority bacterial strains and/or their relative abundance in a donor's stool, wherein the priority bacterial strains are identified in Table 1 as having a 16S V4 sequence of one of SEQ ID NO: 1 to SEQ ID NO: 32.

153. The method of claim 150 or claim 151, wherein the at least one selection criterion comprises the number of priority clusters and/or their relative abundance in a donor's stool, wherein the priority clusters are identified in Table 1 as having a 16S V4 sequence that is at least 97% identical to one of SEQ ID NO: 10, SEQ ID NO: 13, SEQ ID NO: 21, SEQ ID NO: 25, SEQ ID NO: 26, SEQ ID NO: 27, SEQ ID NO: 31, SEQ ID NO: 32, SEQ ID NO: 37, SEQ ID NO: 60, SEQ ID NO: 238, or SEQ ID NO: 240.

154. The method of claim 153 wherein the presence of a priority cluster and its relative abundance in each donor can be determined by counting the number of sequencing reads with more than 97% identity to the priority clusters' sequences identified in Table 1.

155. The method of any one of claims 150 to 154, wherein a donor's stool comprises a least about 5 of the priority bacterial strains and/or the priority clusters identified in Table 1.

156. The method of any one of claims 150 to 155, wherein a donor's stool comprises a least about 7 of the priority bacterial strains and/or the priority clusters identified in Table 1.

157. The method of any one of claims 150 to 156, wherein a donor's stool comprises a least about 12 of the priority bacterial strains and/or the priority clusters identified in Table 1.

158. The method of any one of claims 150 to 157, wherein a donor's stool comprises a relative abundance of priority bacterial strains and/or the priority clusters greater than about 0.01% of the total stool bacterial community.

159. The method of any one of claims 150 to 158, wherein a donor's stool comprises a relative abundance of priority bacterial strains and/or the priority clusters greater than about 0.05% of the total stool bacterial community.

160. The method of any one of claims 150 to 159, wherein a donor's stool comprises a relative abundance of priority bacterial strains and/or the priority clusters greater than about 0.1% of the total stool bacterial community.

161. The method of any one of claims 150 to 160, wherein a donor is selected for being in the top quartile based on the number of priority bacterial strains and/or the priority clusters and abundance thereof in their stool relative to other healthy, screened, pathogen-free potential donors.

162. The method of any one of claims 150 to 161, wherein a donor is selected for being in the top 10th percentile based on the number of priority bacterial strains and/or the priority clusters and abundance thereof in their stool relative to other healthy, screened, pathogen-free potential donors.

163. The method of any one of claims 150 to 162, wherein the at least one selection criterion comprises the presence of one or more of the following bacterial strains in a donor's stool: Bacteria, Actinobacteria, Actinobacteria, Bifidobacteriales, Bifidobacteriaceae, Bifidobacterium; Bacteria, Bacteroidetes, Bacteroidia, Bacteroidales, Bacteroidaceae, Bacteroides; Bacteria, Firmicutes, Bacilli, Lactobacillales, Lactobacillaceae, Lactobacillus; Bacteria, Firmicutes, Bacilli, Lactobacillales, Lactobacillaceae, unclassified; Bacteria, Firmicutes, Clostridia, Clostridiales, Lachnospiraceae, unclassified; Bacteria, Firmicutes, Clostridia, Clostridiales, Ruminococcaceae, Faecalibacterium; Bacteria, Firmicutes, Clostridia, Clostridiales, Ruminococcaceae, unclassified; Bacteria, Firmicutes, Clostridia, unclassified, unclassified, unclassified; Bacteria, Firmicutes, Erysipelotrichia, Erysipelotrichales, Erysipelotrichaceae, Erysipelotrichaceae_incertae_sedis; Bacteria, Firmicutes, Erysipelotrichia, Erysipelotrichales, Erysipelotrichaceae, unclassified; Bacteria, Proteobacteria, Deltaproteobacteria, Desulfovibrionales, Desulfovibrionaceae, Bilophila; and Bacteria, Proteobacteria, unclassified, unclassified, unclassified, unclassified.

164. The method of any one of claims 150 to 163, wherein the one or more bacterial strains comprises two, three, four, five, six, seven, eight, nine, ten, eleven, or twelve bacterial strains.

165. The method of any one of claims 150 to 164, wherein the at least one selection criterion comprises the absence of PSC or the absence of symptoms of PSC.

166. The method of any one of claims 150 to 165, wherein the obtained feces is fresh, frozen, dried, or reconstituted feces.

167. The method of any one of claims 150 to 166, wherein the pharmaceutical composition further comprises at least one bacterial strain that is isolated, purified, and/or cultured.

168. The method any one of claims 150 to 167, wherein the at least bacterial strain comprises a 16S V4 sequence that is greater than about 97% identical to the 16S V4 sequence of any one of the operational taxonomic units (OTUs) recited in Table 1.

169. The method any one of claims 150 to 168, wherein the pharmaceutical composition further comprises a pharmaceutically acceptable excipient.
